SEQUENCE LISTING (113 sequences)

SEQ ID NO: 1 - protein, 18 aa, Artificial Sequence
  Synthetic construct
VAPEEHPTLLTEAPLNPK

SEQ ID NO: 2 - protein, 10 aa, Artificial Sequence
  Synthetic construct
AGFAGDDAPR

SEQ ID NO: 3 - protein, 8 aa, Artificial Sequence
  Synthetic construct
SLLSGLLK

SEQ ID NO: 4 - protein, 10 aa, Artificial Sequence
  Synthetic construct
EGISDGATMK

SEQ ID NO: 5 - protein, 8 aa, Artificial Sequence
  Synthetic construct
SLLAGLLK

SEQ ID NO: 6 - protein, 9 aa, Artificial Sequence
  Synthetic construct
TDGSFIGYK

SEQ ID NO: 7 - protein, 10 aa, Artificial Sequence
  Synthetic construct
LVNEVTEFAK

SEQ ID NO: 8 - protein, 15 aa, Artificial Sequence
  Synthetic construct
DPEGVPPLLVSQQAK

SEQ ID NO: 9 - protein, 11 aa, Artificial Sequence
  Synthetic construct
NCLLTCPGPGR

SEQ ID NO: 10 - protein, 11 aa, Artificial Sequence
  Synthetic construct
GLNQDCCVVYR

SEQ ID NO: 11 - protein, 11 aa, Artificial Sequence
  Synthetic construct
IGTGSFGTVFR

SEQ ID NO: 12 - protein, 10 aa, Artificial Sequence
  Synthetic construct
FATVVEELFR

SEQ ID NO: 13 - protein, 11 aa, Artificial Sequence
  Synthetic construct
GLIPECCAVYR

SEQ ID NO: 14 - protein, 11 aa, Artificial Sequence
  Synthetic construct
DPYGGVAPALK

SEQ ID NO: 15 - protein, 13 aa, Artificial Sequence
  Synthetic construct
TANPILLVSEDQR

SEQ ID NO: 16 - protein, 12 aa, Artificial Sequence
  Synthetic construct
NDVAPTLMSVPR

SEQ ID NO: 17 - protein, 9 aa, Artificial Sequence
  Synthetic construct
TAYFSLDTR

SEQ ID NO: 18 - protein, 9 aa, Artificial Sequence
  Synthetic construct
GPFPQELVR

SEQ ID NO: 19 - protein, 9 aa, Artificial Sequence
  Synthetic construct
LSDPANWLK

SEQ ID NO: 20 - protein, 13 aa, Artificial Sequence
  Synthetic construct
AEETCAPSVSYFK

SEQ ID NO: 21 - protein, 11 aa, Artificial Sequence
  Synthetic construct
AYPDANLLNDR

SEQ ID NO: 22 - protein, 19 aa, Artificial Sequence
  Synthetic construct
AHTPYINIYNCEPANPSEK

SEQ ID NO: 23 - protein, 12 aa, Artificial Sequence
  Synthetic construct
LSGAADTQALLR

SEQ ID NO: 24 - protein, 14 aa, Artificial Sequence
  Synthetic construct
GSKPEDANFYLYLR

SEQ ID NO: 25 - protein, 11 aa, Artificial Sequence
  Synthetic construct
ILGNQGSFLTK

SEQ ID NO: 26 - protein, 19 aa, Artificial Sequence
  Synthetic construct
ADFPTPSISDFEIPTSNIR

SEQ ID NO: 27 - protein, 9 aa, Artificial Sequence
  Synthetic construct
AAEGLDTQR

SEQ ID NO: 28 - protein, 16 aa, Artificial Sequence
  Synthetic construct
DLKPQNLLINTEGAIK

SEQ ID NO: 29 - protein, 10 aa, Artificial Sequence
  Synthetic construct
VVPPLDEDGR

SEQ ID NO: 30 - protein, 16 aa, Artificial Sequence
  Synthetic construct
DLKPENILVTSGGTVK

SEQ ID NO: 31 - protein, 17 aa, Artificial Sequence
  Synthetic construct
VPNGGGGGGGLPISTVR

SEQ ID NO: 32 - protein, 15 aa, Artificial Sequence
  Synthetic construct
ILDVIGLPGEEDWPR

SEQ ID NO: 33 - protein, 17 aa, Artificial Sequence
  Synthetic construct
ALLEAGALPNAPNSYGR

SEQ ID NO: 34 - protein, 12 aa, Artificial Sequence
  Synthetic construct
LPVDLAEELGHR

SEQ ID NO: 35 - protein, 12 aa, Artificial Sequence
  Synthetic construct
DIKPENLLLDER

SEQ ID NO: 36 - protein, 10 aa, Artificial Sequence
  Synthetic construct
ASGPGGGAPR

SEQ ID NO: 37 - protein, 7 aa, Artificial Sequence
  Synthetic construct
AIPELTK

SEQ ID NO: 38 - protein, 11 aa, Artificial Sequence
  Synthetic construct
GSTAENAEYLR

SEQ ID NO: 39 - protein, 10 aa, Artificial Sequence
  Synthetic construct
IPLENLQIIR

SEQ ID NO: 40 - protein, 8 aa, Artificial Sequence
  Synthetic construct
ELVSEFSR

SEQ ID NO: 41 - protein, 8 aa, Artificial Sequence
  Synthetic construct
VGVNGFGR

SEQ ID NO: 42 - protein, 9 aa, Artificial Sequence
  Synthetic construct
LLEYTPTAR

SEQ ID NO: 43 - protein, 15 aa, Artificial Sequence
  Synthetic construct
VGAHAGEYGAEALER

SEQ ID NO: 44 - protein, 13 aa, Artificial Sequence
  Synthetic construct
VNVDEVGGEALGR

SEQ ID NO: 45 - protein, 10 aa, Artificial Sequence
  Synthetic construct
FPYLEASDYK

SEQ ID NO: 46 - protein, 10 aa, Artificial Sequence
  Synthetic construct
LAELEEALQK

SEQ ID NO: 47 - protein, 10 aa, Artificial Sequence
  Synthetic construct
SGFSSVSVSR

SEQ ID NO: 48 - protein, 12 aa, Artificial Sequence
  Synthetic construct
SAYGGPVGAGIR

SEQ ID NO: 49 - protein, 12 aa, Artificial Sequence
  Synthetic construct
SGASEANLIVAK

SEQ ID NO: 50 - protein, 10 aa, Artificial Sequence
  Synthetic construct
LLCTDPGFVK

SEQ ID NO: 51 - protein, 11 aa, Artificial Sequence
  Synthetic construct
LVELEHTLLSK

SEQ ID NO: 52 - protein, 9 aa, Artificial Sequence
  Synthetic construct
SFEDIHHYR

SEQ ID NO: 53 - protein, 14 aa, Artificial Sequence
  Synthetic construct
SGAQASSTPLSPTR

SEQ ID NO: 54 - protein, 11 aa, Artificial Sequence
  Synthetic construct
VAVEEVDEEGK

SEQ ID NO: 55 - protein, 11 aa, Artificial Sequence
  Synthetic construct
VADLVGFLLLK

SEQ ID NO: 56 - protein, 15 aa, Artificial Sequence
  Synthetic construct
LLTQHFVQENYLEYR

SEQ ID NO: 57 - protein, 9 aa, Artificial Sequence
  Synthetic construct
EHTVYGEPR

SEQ ID NO: 58 - protein, 15 aa, Artificial Sequence
  Synthetic construct
LLTQYFVQENYLEYR

SEQ ID NO: 59 - protein, 11 aa, Artificial Sequence
  Synthetic construct
VTDLVQFLLFK

SEQ ID NO: 60 - protein, 11 aa, Artificial Sequence
  Synthetic construct
SGSLVQFLLYK

SEQ ID NO: 61 - protein, 10 aa, Artificial Sequence
  Synthetic construct
YTGYFPVIFR

SEQ ID NO: 62 - protein, 11 aa, Artificial Sequence
  Synthetic construct
VAELVEFLLLK

SEQ ID NO: 63 - protein, 8 aa, Artificial Sequence
  Synthetic construct
LLFFAPTR

SEQ ID NO: 64 - protein, 9 aa, Artificial Sequence
  Synthetic construct
TEFTTALQR

SEQ ID NO: 65 - protein, 8 aa, Artificial Sequence
  Synthetic construct
VADFGLAR

SEQ ID NO: 66 - protein, 10 aa, Artificial Sequence
  Synthetic construct
EGLHSLQEIR

SEQ ID NO: 67 - protein, 9 aa, Artificial Sequence
  Synthetic construct
GQVFDVGPR

SEQ ID NO: 68 - protein, 9 aa, Artificial Sequence
  Synthetic construct
NYLQSLPSK

SEQ ID NO: 69 - protein, 8 aa, Artificial Sequence
  Synthetic construct
IPEQILGK

SEQ ID NO: 70 - protein, 12 aa, Artificial Sequence
  Synthetic construct
VSHKPSGLVMAR

SEQ ID NO: 71 - protein, 14 aa, Artificial Sequence
  Synthetic construct
ISELGAGNGGVVTK

SEQ ID NO: 72 - protein, 12 aa, Artificial Sequence
  Synthetic construct
LFDAPEAPLPSR

SEQ ID NO: 73 - protein, 12 aa, Artificial Sequence
  Synthetic construct
VLGLLGALDPYK

SEQ ID NO: 74 - protein, 12 aa, Artificial Sequence
  Synthetic construct
SNEIPSTDVTDK

SEQ ID NO: 75 - protein, 11 aa, Artificial Sequence
  Synthetic construct
NCLVGANLLVK

SEQ ID NO: 76 - protein, 10 aa, Artificial Sequence
  Synthetic construct
VVSLEEPELR

SEQ ID NO: 77 - protein, 11 aa, Artificial Sequence
  Synthetic construct
LVVVGAGGVGK

SEQ ID NO: 78 - protein, 13 aa, Artificial Sequence
  Synthetic construct
VFSATLGLVDIVK

SEQ ID NO: 79 - protein, 15 aa, Artificial Sequence
  Synthetic construct
VVSEDFLQDVSASTK

SEQ ID NO: 80 - protein, 8 aa, Artificial Sequence
  Synthetic construct
AEGLLQGK

SEQ ID NO: 81 - protein, 11 aa, Artificial Sequence
  Synthetic construct
CAGNEDIITLR

SEQ ID NO: 82 - protein, 8 aa, Artificial Sequence
  Synthetic construct
LQDAGVYR

SEQ ID NO: 83 - protein, 12 aa, Artificial Sequence
  Synthetic construct
ATLLEEQLPLGK

SEQ ID NO: 84 - protein, 12 aa, Artificial Sequence
  Synthetic construct
FQTIPFNVYALK

SEQ ID NO: 85 - protein, 9 aa, Artificial Sequence
  Synthetic construct
GFSGIFEDR

SEQ ID NO: 86 - protein, 7 aa, Artificial Sequence
  Synthetic construct
LFQPFLK

SEQ ID NO: 87 - protein, 9 aa, Artificial Sequence
  Synthetic construct
LINLTDILK

SEQ ID NO: 88 - protein, 10 aa, Artificial Sequence
  Synthetic construct
AVGLLTVISK

SEQ ID NO: 89 - protein, 8 aa, Artificial Sequence
  Synthetic construct
GVTIPSQR

SEQ ID NO: 90 - protein, 10 aa, Artificial Sequence
  Synthetic construct
IYSSNSGPTR

SEQ ID NO: 91 - protein, 11 aa, Artificial Sequence
  Synthetic construct
GLQPECCAVFR

SEQ ID NO: 92 - protein, 9 aa, Artificial Sequence
  Synthetic construct
GYASPDLSK

SEQ ID NO: 93 - protein, 13 aa, Artificial Sequence
  Synthetic construct
VVDPTPEQFQAFR

SEQ ID NO: 94 - protein, 11 aa, Artificial Sequence
  Synthetic construct
LVVVGACGVGK

SEQ ID NO: 95 - protein, 9 aa, Artificial Sequence
  Synthetic construct
SFEDIHQYR

SEQ ID NO: 96 - protein, 9 aa, Artificial Sequence
  Synthetic construct
SFADINLYR

SEQ ID NO: 97 - protein, 10 aa, Artificial Sequence
  Synthetic construct
LLEGEGLPFR

SEQ ID NO: 98 - protein, 9 aa, Artificial Sequence
  Synthetic construct
VFLSPTSLR

SEQ ID NO: 99 - protein, 9 aa, Artificial Sequence
  Synthetic construct
IQDQLQLFR

SEQ ID NO: 100 - protein, 16 aa, Artificial Sequence
  Synthetic construct
AAGDVDIGDAAYYFER

SEQ ID NO: 101 - protein, 12 aa, Artificial Sequence
  Synthetic construct
EVDQQLLSVQTR

SEQ ID NO: 102 - protein, 9 aa, Artificial Sequence
  Synthetic construct
GASALQLER

SEQ ID NO: 103 - protein, 12 aa, Artificial Sequence
  Synthetic construct
ISVYYNEATGGK

SEQ ID NO: 104 - protein, 12 aa, Artificial Sequence
  Synthetic construct
VATYLPAPEGLK

SEQ ID NO: 105 - protein, 10 aa, Artificial Sequence
  Synthetic construct
IQDLLVDSGK

SEQ ID NO: 106 - protein, 15 aa, Artificial Sequence
  Synthetic construct
LLVPANEGDPTETLR

SEQ ID NO: 107 - protein, 11 aa, Artificial Sequence
  Synthetic construct
APLQGTLLGYR

SEQ ID NO: 108 - protein, 12 aa, Artificial Sequence
  Synthetic construct
TATITVLPQQPR

SEQ ID NO: 109 - protein, 16 aa, Artificial Sequence
  Synthetic construct
AVSSFPDPALYPLGSR

SEQ ID NO: 110 - protein, 10 aa, Artificial Sequence
  Synthetic construct
ILLAELEQLK

SEQ ID NO: 111 - protein, 14 aa, Artificial Sequence
  Synthetic construct
SLYASSPGGVYATR

SEQ ID NO: 112 - protein, 9 aa, Artificial Sequence
  Synthetic construct
SPTEPGPER

SEQ ID NO: 113 - protein, 9 aa, Artificial Sequence
  Synthetic construct
GQPFDVGPR

(19) United States
(12) Patent Application Publication
(10) Pub. No.: US 2024/0218454 A1
(43) Pub. Date: Jul. 4, 2024

(54) DIAGNOSTIC TO SUPPORT CLINICAL TRIAL MATCHING AND EXPLORATORY BIOMARKER ANALYSES IN CANCER PATIENTS

(71) Applicant: H. LEE MOFFITT CANCER CENTER AND RESEARCH INSTITUTE, INC., Tampa, FL (US)

(72) Inventors: Eric B. HAURA, Tampa, FL (US); John M. KOOMEN, Tampa, FL (US); Theresa A. BOYLE, Tampa, FL (US); Sudhir PUTTY REDDY, Plainsboro, NJ (US); Aileen Y. ALONTAGA, Temple Terrace, FL (US)

(21) Appl. No.: 18/556,748

(22) PCT Filed: Apr. 22, 2022
(86) PCT No.: PCT/US2022/026057

§ 371 Date: Oct. 23, 2023

Related U.S. Application Data
(60) Provisional application No. 63/178,178, filed on Apr. 22, 2021.

(51) Int. Cl. C12Q 1/6886

(52) U.S. Cl. CPC C12Q 1/6886; C12Q 2600/106; C12Q 2600/158

(57) ABSTRACT

Clinical testing to identify targeted therapies is frequently performed with next generation sequencing (NGS). Clinical action and trial enrollment is often based on expected associations of gene changes with protein expression or action. However, comprehensive RNA and protein expression is not routinely performed in the clinical repertoire of testing clinical tumor samples. The relationship between genetic changes, including gene amplification, with increased RNA and protein expression in the context of clinical testing remains poorly understood. What are needed are new methods of identifying targets that account for both genomic and proteomic information. Disclosed are novel RNA and protein expression panels and methods of using said panels for the detection of cancer and determining the suitability of a subject for a treatment regimen or clinical trial.

This application claims the benefit of U.S. Provisional Application No. 63/178,178, filed on Apr. 22, 2021, which is incorporated herein by reference in its entirety.

### I. BACKGROUND

Clinical testing to identify targeted therapies is frequently performed with next generation sequencing (NGS). Clinical action and trial enrollment is often based on expected associations of gene changes with protein expression or action. However, comprehensive RNA and protein expression is not routinely performed in the clinical repertoire of testing clinical tumor samples. The relationship between genetic changes, including gene amplification, with increased RNA and protein expression in the context of clinical testing remains poorly understood. What are needed are new methods of identifying targets that account for both genomic and proteomic information.

### II. SUMMARY

Disclosed are new genomic and proteomic assays.

In one aspect, disclosed herein are genomic assay panels (such as for example an RNA expression panel, including but not limited to a targeting expression panel such as a Salah targeted expression panel (STEP)) for cancer diagnosis and clinical trial relevance comprising 204 genes selected from the group consisting of ABRAXAS1, ACKR2, ACKR3, ACOT12, ACTA2, ADORA2A, AKT1, AKT2, AKT3, ALK, ANPEP, APC, AR, ARIDIA, ASCL1, ATM, ATR, AXL, B2M, BAG1, BARD1, BCL2, BCL2L11, BIRC5, BRAF, BRCA1, BRCA2, BRIP1, BTN2A1, BTN3A1, CCND1, CCNE1, CD14, CD274, CD33, CD3D, CD3E, CD3G, CD4, CD68, CD70, CD80, CD83, CD86, CD8A, CDH1, CDH2, CDK12, CDK2, CDK4, CDK6, CDKN2A, CHD1, CHEK1, CHEK2, CIITA, CREBBP, CSFIR, CT83, CTAG1A, CTAGE1, CTLA4, CTNNB1, DDR2, DLL3, DUSP4, E2F1, EGFR, ERBB2, ERBB3, ERBB4, ESR1, EZH2, FANCA, FGFR1, FGFR2, FGFR3, FGFR4, FLT1, FLT3, FLT4, FOLR1, FSHR, GATA6, GRB2, GSK3B, HAVCR2, HDAC1, HGF, HLA-A, HLA-B, HLA-C, HLA-DPA1, HLA-DPB1, HLA-DQA1, HLA-DQA2, HLA-DQB1. HLA-DRA, HLA-DRB1, HMMR, HRAS, IDH1, IDH2, IL1 1, IL13RA2, IL2RB, IRS2, JAK2, KDR, KEAP1, KIT, KLRK1, KRAS, LAG3, LCK, MAGEA1, MAGEA10, MAGEA3/6, MAGEA4, MAGEB2, MAGEC1, MAGEC2, MAP2K1, MAP2K2, MAPK1. MAPK3, MCL1, MDM2, MDM4, MET, MET_e14_skip, MICA, MICB, MKI67, MLH1, MRE11, MS4A1, MSH2, MSH6, MTOR, MUC1, MYC, NBN, NCAM1, NEUROD1, NF1, NFE2L2, NFKB1, NFKB2, NRAS, NRG1, NTRK1, NTRK2, NTRK3, OR5V1, PALB2, PARP1, PARP2, PCSK9, PDCD1, PDCDILG2, PDGFRA, PDGFRB, PIK3CA, PMS2, POLE, POU2F3, PPP2R2A, PSCA, PTCH1, PTEN, PTK7, PTP4A1, PVR, RAD51, RAD51B, RAD51C, RAD51D. RAD54L, RAF1, RB1, RET, RICTOR, ROS1, SDC1, SETD2, SIK1, SLC34A2, SMAD4, STK11, TACSTD2, TAFAZZIN, TERT, TFF1, TIGIT, TMPRSS2, TNFRSFIOA, TNFRSF10B, TP53, ULBP1, VIM, WEE1, WT1, and/or YAP1. In some aspects, the panel further comprises one or more housekeeping genes selected from ABCF1, DNAJC14, ERCC3, MRPL19, OAZ1, POLR2A, SMC4, SF3A1, TBC1D10B, TBP, TLK2, and/or TMUB2.

Also disclosed herein are proteomic assay panels for cancer diagnosis and clinical trial relevance comprising the peptide targets comprising ACTA_VAPEEHPTLLTEAPLNPK (SEQ ID NO: 1), ACTB_AGFAGDDAPR (SEQ ID NO: 2), AKT1_SLLSGLLK (SEQ ID NO: 3). AKT2_EGISDGATMK (SEQ ID NO: 4), AKT2_SLLAGLLK (SEQ ID NO: 5), AKT3_TDGSFIGYK (SEQ ID NO: 6), ALBU_LVNEVTEFAK (SEQ ID NO: 7), ALK_DPEGVPPLLVSQQAK (SEQ ID NO: 8), ALK_NCLLTCPGPGR (SEQ ID NO: 9), ARAF_GLNQDCCVVYR (SEQ ID NO: 10), ARAF_IGTGSFGTVFR (SEQ ID NO: 11), BCL2_FATVVEELFR (SEQ ID NO: 12), BRAF_GLIPECCAVYR (SEQ ID NO: 13), BT2A1_DPYGGVAPALK (SEQ ID NO: 14), BT3A_TANPILLVSEDQR (SEQ ID NO: 15), CADH1_NDVAPTLMSVPR (SEQ ID NO: 16), CADH1_TAYFSLDTR (SEQ ID NO: 17), CADH2_GPFPQELVR (SEQ ID NO: 18), CADH2_LSDPANWLK (SEQ ID NO: 19), CCND1_AEETCAPSVSYFK (SEQ ID NO: 20), CCND1_AYPDANLLNDR (SEQ ID NO: 21), CD20_AHTPYINIYNCEPANPSEK (SEQ ID NO: 22), CD3D_LSGAADTQALLR (SEQ ID NO: 23), CD3E_GSKPEDANFYLYLR (SEQ ID NO: 24), CD4_LLGNQGSFLTK (SEQ ID NO: 25), CD80_ADFPTPSISDFEIPTSNIR (SEQ ID NO: 26), CD8A_AAEGLDTQR (SEQ ID NO: 27), CDK2_DLKPQNLLINTEGAIK (SEQ ID NO: 28), CDK2_VVPPLDEDGR (SEQ ID NO: 29), CDK4_DLKPENILVTSGGTVK (SEQ ID NO: 30), CDK4_VPNGGGGGGGLPISTVR (SEQ ID NO: 31), CDK6_ILDVIGLPGEEDWPR (SEQ ID NO: 32), CDN2A_ALLEAGALPNAPNSYGR (SEQ ID NO: 33), CDN2A_LPVDLAEELGHR (SEQ ID NO: 34), CHK1_DIKPENLLLDER (SEQ ID NO: 35), CTGIB_ASGPGGGAPR (SEQ ID NO: 36), CTNB1_AIPELTK (SEQ ID NO: 37), EGFR_GSTAENAEYLR (SEQ ID NO: 38), EGFR_IPLENLQIR (SEQ ID NO: 39), ERBB2_ELVSEFSR (SEQ ID NO: 40), G3P_VGVNGFGR (SEQ ID NO: 41), GSK3B_LLEYTPTAR (SEQ ID NO: 42), HBA_VGAHAGEYGAEALER (SEQ ID NO: 43), HBB_VNVDEVGGEALGR (SEQ ID NO: 44), 113R2_FPYLEASDYK (SEQ ID NO: 45), K2C5_LAELEEALQK (SEQ ID NO: 46), K2C6A_SGFSSVSVSR (SEQ ID NO: 47), K2C7_SAYGGPVGAGIR (SEQ ID NO: 48). KI67_SGASEANLIVAK (SEQ ID NO: 49), KIT_LLCTDPGFVK (SEQ ID NO: 50), KKLC1_LVELEHTLLSK (SEQ ID NO: 51), KRAS_SFEDIHHYR (SEQ ID NO: 52), LMNA_SGAQASSTPLSPTR (SEQ ID NO: 53), LMNA_VAVEEVDEEGK (SEQ ID NO: 54), MAGA1_VADLVGFLLLK (SEQ ID NO: 55), MAGA3_LLTQHFVQENYLEYR (SEQ ID NO: 56), MAGA4_EHTVYGEPR (SEQ ID NO: 57), MAGA6_LLTQYFVQENYLEYR (SEQ ID NO: 58), MAGAA_VTDLVQFLLFK (SEQ ID NO: 59), MAGB2_SGSLVQFLLYK (SEQ ID NO: 60), MAGC1_YTGYFPVIFR (SEQ ID NO: 61), MAGC2_VAELVEFLLLK (SEQ ID NO: 62), MCL1_LLFFAPTR (SEQ ID NO: 63), MET_TEFTTALQR (SEQ ID NO: 64), MET_VADFGLAR (SEQ ID NO: 65), MICA_EGLHSLQEIR (SEQ ID NO: 66), MKO1_GQVFDVGPR (SEQ ID NO: 67), MK03_GQPFDVGPR (SEQ ID NO: 67), MK03_NYLQSLPSK (SEQ ID NO: 68), MP2K I_IPEQILGK (SEQ ID NO: 69), MP2K1_VSHKPSGLVMAR (SEQ ID NO: 70), MP2K2_ISELGAGNGGVVTK (SEQ ID NO: 71), MTOR_LFDAPEAPLPSR (SEQ ID NO: 72), MTOR_VLGLLGALDPYK (SEQ ID NO: 73), NTRK2_SNEIPSTDVTDK (SEQ ID NO: 74), NTRK3_NCLVGANLLVK (SEQ ID NO: 75), NTRK3_VVSLEEPELR (SEQ ID NO: 76), Pan-RAS_LVVVGAGGVGK (SEQ ID NO: 77), PARP1_VFSATLGLVDIVK (SEQ ID NO: 78), PARP1_VVSEDFLQDVSASTK (SEQ ID NO: 79), PARP2_AEGLLQGK (SEQ ID NO: 80), PCNA_CAGNEDIITLR (SEQ ID NO: 81), PD1LI_LQDAGVYR (SEQ ID NO: 82), PDIL2_ATLLEEQLPLGK (SEQ ID NO: 83), PGFRA_FQTIPFNVYALK (SEQ ID NO: 84), PGFRB_GFSGIFEDR (SEQ ID NO: 85), PK3CA_LFQPFLK (SEQ ID NO: 86), PK3CA_LINLTDILK (SEQ ID NO: 87), PSCA_AVGLLTVISK (SEQ ID NO: 88), PTEN_GVTIPSQR (SEQ ID NO: 89), PTEN_IYSSNSGPTR (SEQ ID NO: 90), RAF1_GLQPECCAVFR (SEQ ID NO: 91), RAF1_GYASPDLSK (SEQ ID NO: 92), RAF1_VVDPTPEQFQAFR (SEQ ID NO: 93), RAS ML_LVVVGACGVGK (SEQ ID NO: 94). RASH_SFEDIHQYR (SEQ ID NO: 95), RASN_SFADINLYR (SEQ ID NO: 96), RET_LLEGEGLPFR (SEQ ID NO: 97), RET_VFLSPTSLR (SEQ ID NO: 98), ROS1_IQDQLQLFR (SEQ ID NO: 99), TACD2_AAGDVDIGDAAYYFER (SEQ ID NO: 100), TBB1_EVDQQLLSVQTR (SEQ ID NO: 101), TBB1_GASALQLER (SEQ ID NO: 102), TBB5_ISVYYNEATGGK (SEQ ID NO: 103), TENA_VATYLPAPEGLK (SEQ ID NO: 104), TR10A_IQDLLVDSGK (SEQ ID NO: 105), TR10B_LLVPANEGDPTETLR (SEQ ID NO: 106), UFO_APLQGTLLGYR (SEQ ID NO: 107), UFO_TATITVLPQQPR (SEQ ID NO: 108), VGFR1_AVSSFPDPALYPLGSR (SEQ ID NO: 109), VIME_LLLAELEQLK (SEQ ID NO: 110), VIME_SLYASSPGGVYATR (SEQ ID NO: 111), and/or WEE1_SPTEPGPER (SEQ ID NO: 112).

In one aspect, disclosed herein are methods of measuring the suitability of a patient for a treatment regimen, the appropriate treatment for a subject, or clinical trial comprising: obtaining a tissue sample from the subject (including, but not limited to blood, serum, peripheral blood mononuclear cells (PBMC), stool, urine, saliva, sputum, tissue resection, and/or core biopsy); assaying gene expression in a tumor cell in the biological sample using the gene expression panel of any preceding aspect; and/or assaying the protein expression of in a tumor cell in the biological sample using protein expression panel of any preceding aspect; wherein the expression of or a modulation in expression of at least 1, 2, 3, 4, 5, 6, 7 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114,115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or 204 genes and/or the expression of or a modulation in expression of at least 1, 2, 3, 4, 5, 6, 7 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112 proteins; or the pattern of gene and/or protein expression (i.e., which genes or proteins are expressed and/or modulated) indicates the suitability of a patient for a treatment regimen, the appropriate treatment for a subject, or clinical trial.

In some aspects, the tissue sample can be fresh or frozen (including formalin fixed paraffin embedded samples).

Also disclosed herein are methods of measuring the suitability of a patient for a treatment regimen, the appropriate treatment for a subject, or clinical trial of any preceding aspect, wherein the gene expression panel is measured using a multiplexed polymerase chain reaction assay on the expression panel or nanostring RNA expression profiling.

Also disclosed herein are methods of measuring the suitability of a patient for a treatment regimen, the appropriate treatment for a subject, or clinical trial of any preceding aspect, wherein protein expression is measured mass spectrometry (such as, for example, liquid chromatography multiple reaction monitoring (LC_MRM)).

In some aspect, disclosed herein are methods of measuring the suitability of a patient for a treatment regimen, the appropriate treatment for a subject, or clinical trial of any preceding aspect, wherein the method further comprises treating the subject with an anti-cancer agent.

### III. BRIEF DESCRIPTION OF THE DRAWINGS

The accompanying drawings, which are incorporated in and constitute a part of this specification, illustrate several embodiments and together with the description illustrate the disclosed compositions and methods.

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-G shows the analytical sensitivity/limit of detection. (A) Limit of detection: analysis of 2 serially diluted samples displaying 80% tumor cellularity or 20% tumor cellularity. (B) Sample P6-9 with 80% tumor cellularity. (C) Sample P1-1 with 20% tumor cellularity. (D-E) Correlation of log 2 ratio with different input amounts. (D) P6-9, 80% tumor cellularity; (E) P1-1, 20% tumor cellularity. (F) Samples with low tumor cellularity and low RNA amount (amplifications). (G) Samples with low cellularity and Met exon 14 skipping.

FIG. 2A-F shows the precision. (A) Pearson correlations between different comparisons. (B) NGS and RNA STEP concordance (different operators). (C) Pearson correlation of universal RNA controls in 12 different runs. (D) STAR NGS and RNA STEP concordance (different nCounter machine). (E) STAR NOS and RNA STEP concordance (different thermocycler machine). (F) STAR NGS and RNA STEP concordance (different lot number).

FIG. 3A-C shows the specificity/Interfering substance. (A) Samples tested to evaluate for interfering substances. (B) Sample age. (C) Effect of background tissue type.

FIG. 4A-E shows the concordance between STAR NGS and RNA STEP. (A) Different cut-offs for STAR NGS and RNA STEP. (B) Specific genes accuracy, PPA, NPA, PPV, NPV. (C) HER2 positive cases by STAR NGS and tested with RNA STEP. Samples with Met exon 14 skipping. (D) Met exon 14 skipping PPA, NPA, PPV, NPV, and Accuracy. (E) Histogram of the correlation between RNA STAR and RNAseq.

FIG. 5 shows NanoString Elements TagSet Hybridization reaction.

FIG. 6 shows a Venn Diagram showing the number of overlapping genes in the NOS, Custom, and TS360 Panels.

FIG. 7 shows PPA between the different panels.

FIG. 8 shows Pearson correlation between Run 1 vs Run 2 of three different lung cancer patient RNA samples. Log 2 Fold change (FC) is the ratio of the sample gene over the lung non-tumor control sample.

FIG. 9 shows Pearson correlation between 6 different runs of the lung non-tumor control sample.

FIG. 10 shows the assay development includes selection of clinically relevant biomarkers as well as unique proteolytic peptides for each biomarker. “Light” and stable isotope labeled standard (SIS) peptides were analyzed with LC-MS/MS to select fragment ions and optimize their collision energies to maximize detection. Reverse calibration curves were used to evaluate the assay sensitivity and linearity.

FIG. 11 shows the optimized protocol for FFPE sample processing for LC-MS/MS (left) and the examples of discovery proteomics data of two different tumors (right).

FIG. 12 shows the list of biomarkers in the LC-MRM assay panel.

FIG. 13A-F shows NSCLC cell line digests were prepared for proteome analysis. Similar numbers of proteins and peptides were observed for all cell lines in discovery proteomics without fractionation. (A) A matrix was prepared using equal amounts of tryptic digests of 25 NSCLC cell lines. In the background, reverse calibration curves (RCCs) were established for each peptide from the lung cancer biomarkers. Five representative examples are shown (B-F).

FIG. 14 shows the optimized collision energies of selected proteolytic peptides (n=141) are shown with their m/z values. These data show agreement between different sets of assays developed over time in the lab.

FIG. 15 shows the heat map of protein expression across 25 NSCLC cell lines quantified by SRM assays. Visualization of protein expression levels is shown with red, white, and blue from high to low. Gray indicates undetected protein levels in NSCLC cells. Some proteins are consistently detected, while others, like cancer antigens, are less frequently observed.

FIG. 16A-C shows multiplexed targeted proteomics assay development to quantitate 97 lung cancer biomarkers. The summary of assay platform performance. (A) Scatter plot shows the Lower limits of quantification (LLOQ) of 137 peptides from 97 proteins determined from reverse calibration curves. A median LLOQ of 159 amol was observed for 137 peptides, where 88.3% (121 of 137) of peptides showed LLOQs <500 amol. The coefficient of variation (CV) values for intra-day (B) and inter-day (C) repeatability experiments were below 20% for 120 and 120, 130 and 127, 136 and 136 peptides at the low (500 amol), medium (2.5 fmol), and high (12.5 fmol) amounts of spiked peptides on column.

FIG. 17A-D shows LC-MRM analysis of lysates from 25 NSCLC cell lines shows distinct expression of biomarkers in concordance with known protein levels. Quantitative data in amol/microgram of total protein digest were visualized in a heat map with clustering of both the NSCLC cell lines and the peptide measurements (A). The examples of peptide measurements for EGFR (B), MET (C), and Cyclin D1 (D) were presented as bar graphs to show the agreement between peptides, detectability in cell lines and the distribution of expression.

FIG. 18A-D shows LC-MRM protein biomarker quantification in FFPE NSCLC lung tumors to show compatibility with biopsy specimens. Tumor tissues were laser capture microdissected to exclude adjacent lung tissue and processed using filter-aided sample preparation (FASP-add citation). Total recovered peptide amounts from Nanodrop assays (need to be specific about which assay) were plotted against the tissue area in square millimeters to determine the amounts of protein recovery from these sections (A); three sections of 5 micron thickness were combined for this experiment. Aliquots (1 microgram total protein digest) were analyzed with LC-MS/MS to evaluate the effectiveness of the digestion and the consistency of protein and peptide identification (B). Principal component analysis was used to determine any samples that were outliers in the LC-MS/MS data (C). LC-MRM data were visualized as a heat map to examine clustering of tumor types (D).

FIG. 19 shows the application of LC-MRM assay of 108 frozen tissue specimens from lung squamous cell carcinoma patients showing four subtypes.

FIG. 20 shows the triplicate measurements of lung cancer cell lines showing strong correlation. Heat map and cluster dendrograms (A) of individual replicate measurements of 25 lung cancer cell lines. Scatter plots with trendlines and correlation calculations show strong agreement between replicate 2 and replicate 1 (B) as well as replicate # and replicate #(C).

FIG. 21 shows the duplicate measurements of FFPE NSCLC lung tumors showing high consistency. Heat map and cluster dendrograms (A) of individual replicate measurements of 8 of the FFPE lung tumors specimens. Scatter plots with trendlines and correlation calculations show strong agreement between replicate 2 and replicate 1 in linear scale (B) and as log 2 transformed data to better display low abundance peptide measurements and the range of individual measurements that were not observed in both replicates (C).

FIG. 22 shows the application of LC-MRM of 108 frozen tissue specimens from lung squamous cell carcinoma patients phenotyping panel.

FIG. 23 shows the application of LC-MRM of 108 frozen tissue specimens from lung squamous cell carcinoma patients targeted therapy panel.

FIG. 24 shows the application of LC-MRM of 108 frozen tissue specimens from lung squamous cell carcinoma patients immunotherapy panel.

FIG. 25A-F shows the targeted proteomics of frozen tissues from lung squamous cell carcinoma patients (n=108) distinguishes tumor subtypes. LC-MRM quantitation of biomarkers and PCA analysis differentiated the inflamed and redox LSCC tumors (A) as well as the tumors expressing high and low levels of glycolytic enzyme, GAPDH (B). The tumor subtyping shown in FIG. 4A was performed based on the previous report, Stewart et al. Nat. Commun 2019, 10, 3578. Volcano plots show the proteins identified with significant differential regulation in the comparisons of inflamed/redox tumors (C) and High/Low GAPDH expressing tumors (D). Box and Whisker plots represent selected proteins with significant differences in expression between the LSCC tumor subtypes (E-F).

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.” “Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

“Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### A. COMPOSITIONS

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular genomic or proteomic assay panel is disclosed and discussed and a number of modifications that can be made to a number of molecules including the genomic or proteomic assay panel are discussed, specifically contemplated is each and every combination and permutation of genomic or proteomic assay panel and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Herein, we report the concordance between gene amplification and RNA expression by comparing results from testing samples for gene amplification by NGS and RNA expression with a custom 204 gene RNA expression panel, RNA STEP (Salah Targeted Expression Panel).

Moffitt Cancer Center has multiple and changing clinical trials for patients with cancer, many with biomarker-based inclusion and exclusion criteria. We elicited feedback from Moffitt clinicians about which genes are most needed for clinical trial screening and designed a custom gene panel of 204 test and 12 housekeeping genes. RNA expression was performed with the NanoString NCounter platform, an amplification-free, multiplexed RNA profiling technology that is optimized for mRNA extracted from FFPE samples.

In one aspect, disclosed herein are genomic assay panels (such as for example an RNA expression panel, including but not limited to a targeting expression panel such as a Salah targeted expression panel (STEP)) for cancer diagnosis and clinical trial relevance comprising 204 genes selected from the group consisting of ABRAXAS1, ACKR2, ACKR3, ACOT12, ACTA2, ADORA2A, AKT1, AKT2, AKT3, ALK, ANPEP, APC, AR, ARIDIA, ASCL1, ATM, ATR, AXL, B2M, BAG1, BARD1, BCL2, BCL2L11, BIRC5, BRAF, BRCA1, BRCA2, BRIP1, BTN2A1, BTN3A1, CCND1, CCNE1, CD14, CD274, CD33, CD3D, CD3E, CD3G, CD4, CD68, CD70, CD80, CD83, CD86, CD8A, CDH1, CDH2, CDK12, CDK2, CDK4, CDK6, CDKN2A, CHD1, CHEK1, CHEK2, CIITA, CREBBP, CSF1R, CT83, CTAGIA, CTAGE1, CTLA4, CTNNB1, DDR2, DLL3, DUSP4, E2F1, EGFR, ERBB2, ERBB3, ERBB4, ESR1, EZH2, FANCA, FGFR1, FGFR2, FGFR3, FGFR4, FLT1, FLT3, FLT4, FOLR1, FSHR, GATA6, GRB2, GSK3B, HAVCR2, HDAC1, HGF, HLA-A, HLA-B, HLA-C, HLA-DPA1, HLA-DPB1, HLA-DQA1, HLA-DQA2, HLA-DQB1, HLA-DRA, HLA-DRB1, HMMR, HRAS. IDH1, IDH2, IL11. IL13RA2, IL2RB, IRS2, JAK2, KDR, KEAP1, KIT, KLRK1, KRAS, LAG3, LCK, MAGEA1, MAGEA10, MAGEA3/6, MAGEA4, MAGEB2, MAGEC1, MAGEC2, MAP2K1, MAP2K2, MAPK1, MAPK3, MCL1. MDM2, MDM4, MET, MET_e14_skip, MICA, MICB, MKI67, MLH1, MRE11, MS4A1, MSH2, MSH6, MTOR, MUC1, MYC, NBN, NCAM1, NEUROD1, NF1, NFE2L2, NFKB1, NFKB2, NRAS, NRG1, NTRK1, NTRK2, NTRK3, OR5V1, PALB2, PARP1, PARP2, PCSK9, PDCD1, PDCD1LG2, PDGFRA, PDGFRB, PIK3CA, PMS2, POLE, POU2F3, PPP2R2A, PSCA, PTCH1, PTEN, PTK7, PTP4A1, PVR, RAD51, RAD5IB, RAD51C, RAD51D. RAD54L, RAF1, RB1, RET, RICTOR, ROS1, SDC1, SETD2, SIK1, SLC34A2, SMAD4, STK11, TACSTD2, TAFAZZIN, TERT, TFF1, TIGIT, TMPRSS2, TNFRSFIOA, TNFRSF10B, TP53, ULBP1, VIM, WEE1, WT1, and/or YAP1. In some aspects, the panel further comprises one or more housekeeping genes selected from ABCF1, DNAJCl4, ERCC3, MRPL19, OAZ1, POLR2A, SMC4, SF3A1, TBC1D10B, TBP, TLK2, and/or TMUB2.

Also disclosed herein are proteomic assay panels for cancer diagnosis and clinical trial relevance comprising the peptide targets comprising ACTA_VAPEEHPTLLTEAPLNPK (SEQ 1D NO: 1), ACTB_AGFAGDDAPR (SEQ ID NO: 2), AKT1_SLLSGLLK (SEQ ID NO: 3), AKT2_EGISDGATMK (SEQ ID NO: 4), AKT2_SLLAGLLK (SEQ ID NO: 5), AKT3_TDGSFIGYK (SEQ ID NO: 6), ALBU_LVNEVTEFAK (SEQ ID NO: 7), ALK_DPEGVPPLLVSQQAK (SEQ ID NO: 8), ALK_NCLLTCPGPGR (SEQ ID NO: 9), ARAF_GLNQDCCVVYR (SEQ ID NO: 10), ARAF_IGTGSFGTVFR (SEQ ID NO: 11), BCL2_FATVVEELFR (SEQ ID NO: 12), BRAF_GLIPECCAVYR (SEQ ID NO: 13). BT2A1_DPYGGVAPALK (SEQ ID NO: 14), BT3A1_TANPILLVSEDQR (SEQ ID NO: 15), CADH1_NDVAPTLMSVPR (SEQ ID NO: 16), CADH1_TAYFSLDTR (SEQ ID NO: 17), CADH2_GPFPQELVR (SEQ ID NO: 18), CADH2_LSDPANWLK (SEQ ID NO: 19). CCND1_AEETCAPSVSYFK (SEQ ID NO: 20), CCND1_AYPDANLLNDR (SEQ ID NO: 21), CD20_AHTPYINIYNCEPANPSEK (SEQ ID NO: 22), CD3D_LSGAADTQALLR (SEQ ID NO: 23), CD3E_GSKPEDANFYLYLR (SEQ ID NO: 24), CD4_ILGNQGSFLTK (SEQ ID NO: 25), CD80_ADFPTPSISDFEIPTSNIR (SEQ ID NO: 26), CD8A_AAEGLDTQR (SEQ ID NO: 27), CDK2_DLKPQNLLINTEGAIK (SEQ ID NO: 28), CDK2_VVPPLDEDGR (SEQ ID NO: 29), CDK4_DLKPENILVTSGGTVK (SEQ ID NO: 30). CDK4_VPNGGGGGGGLPISTVR (SEQ ID NO: 31), CDK6_ILDVIGLPGEEDWPR (SEQ ID NO: 32), CDN2A_ALLEAGALPNAPNSYGR (SEQ ID NO: 33), CDN2A_LPVDLAEELGHR (SEQ ID NO: 34), CHK1_DIKPENLLLDER (SEQ ID NO: 35), CTG1B_ASGPGGGAPR (SEQ ID NO: 36), CTNB1_AIPELTK (SEQ ID NO: 37), EGFR_GSTAENAEYLR (SEQ ID NO: 38), EGFR_IPLENLQIIR (SEQ ID NO: 39), ERBB2_ELVSEFSR (SEQ ID NO: 40), G3P_VGVNGFGR (SEQ ID NO: 41), GSK3B_LLEYTPTAR (SEQ ID NO: 42), HBA_VGAHAGEYGAEALER (SEQ ID NO: 43), HBB_VNVDEVGGEALGR (SEQ ID NO: 44), Il3R2_FPYLEASDYK (SEQ ID NO: 45), K2C5_LAELEEALQK (SEQ ID NO: 46), K2C6A_SGFSSVSVSR (SEQ ID NO: 47), K2C7_SAYGGPVGAGIR (SEQ ID NO: 48), K167_SGASEANLIVAK (SEQ ID NO: 49), KIT_LLCTDPGFVK (SEQ ID NO: 50), KKLC1_LVELEHTLLSK (SEQ ID NO: 51), KRAS_SFEDIHHYR (SEQ ID NO: 52), LMNA_SGAQASSTPLSPTR (SEQ ID NO: 53). LMNA_VAVEEVDEEGK (SEQ ID NO: 54), MAGA1_VADLVGFLLLK (SEQ ID NO: 55), MAGA3_LLTQHFVQENYLEYR (SEQ ID NO: 56), MAGA4_EHTVYGEPR (SEQ ID NO: 57), MAGA6_LLTQYFVQENYLEYR (SEQ ID NO: 58), MAGAA_VTDLVQFLLFK (SEQ ID NO: 59), MAGB2_SGSLVQFLLYK (SEQ ID NO: 60), MAGC1_YTGYFPVIFR (SEQ ID NO: 61), MAGC2_VAELVEFLLLK (SEQ ID NO: 62), MCL1_LLFFAPTR (SEQ ID NO: 63), MET_TEFTTALQR (SEQ ID NO: 64), MET_VADFGLAR (SEQ ID NO: 65), MICA_EGLHSLQEIR (SEQ ID NO: 66), MK01_GQVFDVGPR (SEQ ID NO: 67), MK03_GQPFDVGPR (SEQ ID NO: 67), MK03_NYLQSLPSK (SEQ ID NO: 68), MP2K1_IPEQ1LGK (SEQ ID NO: 69), MP2K1_VSHKPSGLVMAR (SEQ ID NO: 70), MP2K2_ISELGAGNGGVVTK (SEQ ID NO: 71), MTOR_LFDAPEAPLPSR (SEQ ID NO: 72), MTOR_VLGLLGALDPYK (SEQ ID NO: 73), NTRK2_SNEIPSTDVTDK (SEQ ID NO: 74), NTRK3_NCLVGANLLVK (SEQ ID NO: 75), NTRK3_VVSLEEPELR (SEQ ID NO: 76), Pan-RAS_LVVVGAGGVGK (SEQ ID NO: 77), PARP1_VFSATLGLVDIVK (SEQ ID NO: 78), PARP1_VVSEDFLQDVSASTK (SEQ ID NO: 79), PARP2_AEGLLQGK (SEQ ID NO: 80), PCNA_CAGNEDIITLR (SEQ ID NO: 81), PD1L1_LQDAGVYR (SEQ ID NO: 82), PD1L2_ATLLEEQLPLGK (SEQ ID NO: 83), PGFRA_FQTIPFNVYALK (SEQ ID NO: 84), PGFRB_GFSGIFEDR (SEQ ID NO: 85), PK3CA_LFQPFLK (SEQ ID NO: 86), PK3CA_LINLTDILK (SEQ ID NO: 87), PSCA_AVGLLTVISK (SEQ ID NO: 88), PTEN_GVTIPSQR (SEQ ID NO: 89), PTEN_IYSSNSGPTR (SEQ ID NO: 90), RAF1_GLQPECCAVFR (SEQ ID NO: 91), RAF1_GYASPDLSK (SEQ ID NO: 92), RAF1_VVDPTPEQFQAFR (SEQ ID NO: 93), RAS Mt_LVVVGACGVGK (SEQ ID NO: 94), RASH_SFEDIHQYR (SEQ ID NO: 95), RASN_SFADINLYR (SEQ ID NO: 96), RET_LLEGEGLPFR (SEQ ID NO: 97), RET_VFLSPTSLR (SEQ ID NO: 98), ROS1_IQDQLQLFR (SEQ ID NO: 99), TACD2_AAGDVDIGDAAYYFER (SEQ ID NO: 100), TBB1_EVDQQLLSVQTR (SEQ ID NO: 101), TBB1_GASALQLER (SEQ ID NO: 102), TBB5_ISVYYNEATGGK (SEQ ID NO: 103), TENA_VATYLPAPEGLK (SEQ ID NO: 104). TR10A_IQDLLVDSGK (SEQ ID NO: 105). TRIOB_LLVPANEGDPTETLR (SEQ ID NO: 106), UFO_APLQGTLLGYR (SEQ ID NO: 107), UFO_TATITVLPQQPR (SEQ ID NO: 108), VGFR1_AVSSFPDPALYPLGSR (SEQ ID NO: 109), VIME_ILLAELEQLK (SEQ ID NO: 110), VIME_SLYASSPGGVYATR (SEQ ID NO: 111), and/or WEE1_SPTEPGPER (SEQ ID NO: 112).

### 1. Homology/Identity

It is understood that one way to define any known variants and derivatives or those that might arise, of the disclosed genes and proteins herein is through defining the variants and derivatives in terms of homology to specific known sequences. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 2. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### 3. Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1.8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-1 methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO^{T}M-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy Fl; Bodipy FL ATP; Bodipy Fl-Ceramide; Bodipy R6G SE; Bodipy TMR; Bodipy TMR-X conjugate; Bodipy TMR-X. SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcotluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18(5)); DIDS; Dihydorhodamine 123 (DHR); Dil (DiIC18(3)); I Dinitrophenol; DiO (DiOC18(3)); DiR; DiR (DiIC18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type' non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1, high calcium; Indo-1 low calcium; indodicarbocyanine (DiD); indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow: Nylosan Brilliant lavin E8G; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (Pl); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 60; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C: S65L; S65T; Sapphire GFP; SBFI; Serotonin; Sevron Brilliant Red 2B; Sevron Brilliant Red 40; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62: SYTO 63; SYTO 64; SYTO 80; SYTO 81; SYTO 82; SYTO 83: SYTO 84: SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thiotlavin 5; Thioflavin S; Thioflavin TON; Thiolyte; Thiozole Orange; Tinopol CBS (Calcofluor White); TIER; TO—PRO-1; TO-PRO-3; TO-PRO-5; TOTO-1; TOTO-3; TriColor (PE-Cy5); TRITC TetramethylRodaminelsoTMioCyanate; True Blue: Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the apset include, but are not limited to, Tc-99m, Re-186, Ga-68, Re-188, Y-90, Sm-153, Bi-212. Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group I assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10 MW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods. Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U.K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis-I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, P-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, i-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction. NJ) and specialised chip designs, such as engineered microchannels in a plate (e.g., The Living Chip™, Biotrove, Woburn, MA) and tiny 3D posts on a silicon surface (Zyomyx, Hayward CA). Particles in suspension can also be used as the basis of arrays, providing they are coded for identification; systems include colour coding for microbeads (Luminex, Austin, TX; Bio-Rad Laboratories) and semiconductor nanocrystals (e.g., QDots™, Quantum Dot, Hayward, CA), and barcoding for beads (UltraPlex™, SmartBead Technologies Ltd, Babraham, Cambridge, UK) and multimetal microrods (e.g., Nanobarcodes™ particles, Nanoplex Technologies, Mountain View, CA). Beads can also be assembled into planar arrays on semiconductor chips (LEAPS technology, BioArray Solutions, Warren, NJ).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, WA) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, MA), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilised on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm. at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Inuinsic Bioprobes, Tempe, AZ), rolling circle DNA amplification (Molecular Staging, New Haven CT), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, CA; Clontech, Mountain View, CA; BioRad; Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; Biolnvent, Lund, Sweden; Affitech, Walnut Creek, CA; Biosite, San Diego, CA). In addition to the conventional antibodies, Fab and scFv fragments, single V-domains from camelids or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, CO). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colours. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (ProteinPrint™, Aspira Biosystems, Burlingame, CA).

Another methodology which can be used diagnostically and in expression profiling is the ProteinChip® array (Ciphergen, Fremont, CA), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (Proteometrix, Branford, CT).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### B. METHODS OF ASSESSING A TREATMENT REGIMEN OR IDENTIFYING THE APPROPRIATE TREATMENT FOR A SUBJECT

In one aspect, disclosed herein are methods of measuring the suitability of a patient for a treatment regimen, the appropriate treatment for a subject, or clinical trial comprising: obtaining a tissue sample from the subject (including, but not limited to blood, serum, peripheral blood mononuclear cells (PBMC), stool, urine, saliva, sputum, tissue resection, and/or core biopsy); assaying gene expression in a tumor cell in the biological sample using the gene expression panel of any preceding aspect; and/or assaying the protein expression of in a tumor cell in the biological sample using protein expression panel of any preceding aspect; wherein the expression of or a modulation in expression of at least 1, 2, 3, 4, 5, 6, 7 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129,130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, or 204 genes and/or the expression of or a modulation in expression of at least 1, 2, 3, 4, 5, 6, 7 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112 proteins. In some instances cases the increased expression of a gene or protein will be indicative of the suitability of a treatment regiment or be indicative of an appropriate treatment. In some instances cases the decreased expression of a gene or protein will be indicative of the suitability of a treatment regimen or be indicative of an appropriate treatment.

It is understood and herein contemplated that it is not always the number of the genes or proteins expressed in the assay that is determinative of the treatment regimen but can also depend on which genes or proteins are expressed or the reduced of expression of a gene or protein (i.e., the gene and/or protein expression pattern). In particular, the expression of genes or proteins associated with a particular cancer, susceptibility to a particular treatment regimen or resistance to a particular treatment regimen can also be assessed.

In some aspects, the tissue sample can be fresh or frozen (including formalin fixed paraffin embedded samples).

In one aspect disclosed herein are methods of measuring the suitability of a patient for a treatment regimen, the appropriate treatment for a subject, or clinical trial, wherein the gene expression panel is measured using a multiplexed polymerase chain reaction assay on the expression panel or nanostring RNA expression profiling.

Also disclosed herein are methods of measuring the suitability of a patient for a treatment regimen, the appropriate treatment for a subject, or clinical trial, wherein protein expression is measured mass spectrometry (such as, for example, liquid chromatography multiple reaction monitoring (LC_MRM)).

It is understood and herein contemplated that the disclosed genomic and proteomic assays and expression panels can be used to assess the suitability of treatment or the appropriate treatment regimen for a subject with any disease where uncontrolled cellular proliferation occurs such as cancers. A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer, lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer, prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer, genitourinary cancer; pulmonary cancer; esophageal carcinoma; head and neck carcinoma; large bowel cancer; hematopoietic cancers; testicular cancer; and colon and rectal cancers.

In some aspects the disclosed methods of measuring the suitability of a patient for a treatment regimen or clinical trial can further comprises treating the subject with an anti-cancer agent. It is understood and herein contemplated that the disclosed treatment regimens can used alone or in combination with any anti-cancer therapy known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus). Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride). Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene. Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (ifosfamide), Cyramza (Ramucirumab), Cytarabine, Cytarabine Liposome, Cytosar-U (Cytarabine), Cytoxan (Cyclophosphamide). Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab. DepoCyt (Cytarabine Liposome), Dexamethasone, Dexrazoxane Hydrochloride, Dinutuximab, Docetaxel. Doxil (Doxorubicin Hydrochloride Liposome), Doxorubicin Hydrochloride, Doxonibicin Hydrochloride Liposome, Dox-SL (Doxorubicin Hydrochloride Liposome), DTIC-Dome (Dacarbazine). Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faskxlex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI. FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide). IL-2 (Aldesleukin), Imatinib Mesylate, Imbruvica (lbrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine 1131 Tositumomab and Tositumomab, Ipilimumab, Iressa (Gefitinib), Irinotecan Hydrochloride, Irinotecan Hydrochloride Liposome, Istodax (Romidepsin), ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium. Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride. Mercaptopurine, Mesna, Mesnex (Mesna), Methazolastone (Temozolomide). Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C) Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide). Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim). Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin). Paraplatin (Carboplatin). Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab. Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V. Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus. Thalidomide, Thalomid (Thalidomide), Thioguanine. Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (Nivolumab (BMS-936558 or MDX1106), CT-011, MK-3475), PD-L1 (MDX-1 105 (BMS-936559), MPDL3280A, or MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### C. EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric. There are numerous variations and combinations of process conditions that can be used to optimize product quality and performance. Only reasonable and routine experimentation will be required to optimize such process conditions.

### 1. Example 1

### 2. Validation of a Moffitt Custom Tumor RNA Expression Panel to Support Clinical Trial Matching

BACKGROUND: Successful matching of patients with cancer to clinical trials can be challenging and considered to be improved with clinical information about RNA gene expression. The aim is to validate a Custom Tumor RNA Expression Panel in the CLIA laboratory to enable pre-screening of patients with cancer for clinical trials. A secondary goal is to support trial exploratory biomarker analyses. The Custom RNA Panel was designed based on feedback from Moffitt clinicians about which genes are most needed for clinical trial prescreening.

METHODS: The Custom RNA Panel consists of reagents for testing of 216 (204 test and 12 housekeeping) genes using the NanoString platform. NanoString is an amplification-free, multiplexed RNA profiling technology that is optimized for mRNA extracted from FFPE samples. In the exploratory validation phase, a non-tumor lung control and 19 remnant RNA lung cancer specimens with at least one gene amplification (cut-off≥4 copies) reported by testing with the Moffitt STAR next generation sequencing (NGS. Illumina TST170) platform was tested. Three specimens were repeated. All samples were also tested with a commercially available NanoString panel of 760 genes (TS360). STAR NGS copies versus RNA expression fold change were analyzed for positive percentage agreement (PPA) using pre-defined cut-offs and compared results with Pearson correlation.

RESULTS: The 19 selected clinical samples had 13 genes with amplification of ≥5 copies and 6 genes with 4 copies. Of the 13 genes with ≥5 copies by NGS, all had increased fold change by the RNA expression panel (cut-off ≥1-fold increased expression, 100% PPA). Of 6 genes with 4 copies, 2 had high gene expression (33.3% PPA). Results from 3 repeated clinical samples demonstrated high reproducibility (r=0.99). Results of overlapping genes from the Custom versus TS360 RNA expression panels were strongly correlated (r=0.94).

CONCLUSION: The initial performance of the Moffitt Custom RNA Expression Panel exhibited high concordance with NGS amplification results and good reproducibility with repeated samples. Remnant RNA from clinical genetic testing can be tested with the Custom RNA Expression Panel to provide innovative clinical information about patients' cancers and CLIA validation facilitates clinical trial prescreening.

### 3. Example 2

### Methods for Sample Preparation and Targeted Proteomics of the Biomarker Panel.

Preparation of Cell Lines or Frozen Tumor Tissues. Cell pellets or frozen pulverized tumor tissues are resuspended in denaturing lysis buffer containing aqueous 20 mM HEPES, pH 8.0, 9 M urea, 1 mM sodium orthovanadate, 2.5 mM sodium pyrophosphate, and 1 mM β-glycerophosphate. After vortexing and brief sonication, the lysates are cleared by centrifugation at 13,000×g for 15 minutes at 15° C. Protein concentration is estimated by Bradford Assay (Coomassie Plus. Pierce). The proteins are reduced with 5 mM dithiothreitol (DTT) at 29° C. for 30 minutes followed by alkylation with 10 mM iodoacetamide (IAA) in the dark at room temperature for 30 minutes. The samples are diluted to a final concentration of 1.5 M urea, and trypsin digestion was carried out at 37° C. for 16 hours with an enzyme/substrate (w/w) ratio of 1/25. Stable isotope labeled standard (SIS) or “heavy” peptides are added at known concentration to enable quantification of the proteolytic peptides from the endogenous proteins. Peptides are extracted using reversed phased media in pipette tips (C18 Ziptip. MilliporeSigma) or cartridges (SepPak. Waters), depending on the total quantity of protein digest. For frozen tissues, the protein extraction using 9 M urea buffer and enzymatic digestion are similar to the process described above for cell lines.

Sample Preparation for Formalin Fixed Paraffin Embedded Archival Tumor Specimens. The proteins were extracted from FFPE tissues using SDS lysis buffer (containing 1.5% SDS, 50 mM DTT, 100 mM Tris-HCl, pH 7.6) and digested using the filter aided sample processing (FASP) approach as described previously. Briefly, 5 μm sections with sizes ranging from 3-430 mm² were sectioned with a microtome and collected in Eppendorf tubes (4 slices per patient). FFPE sections (n=3) were deparaffinized by washing with 1 mL Sub-X (xylene substitute) three times, and then rehydrated in two steps with each of the following: 1 mL of 100%, 85%, and 70% ethanol for 5 minutes, and finally with 1 mL of water for 1 minute. SDS lysis buffer was added, and the sections were sonicated using a Bioruptor (15 cycles, each cycle 20 s), heated for 90 minutes at 95° C., and sonicated again (for 15 additional cycles). Then, the SDS buffer was exchanged with urea buffer (8 M urea, 1 mM DTT, 100 mM Tris-HCl, pH 8.5) using s 30 kDa molecular weight cutoff filters (Merck, Millipore) and the proteins were alkylated using aqueous 50 mM IAA, 8 M Urea, 100 mM Tris-HCl, 1 mM DTT. pH 8.5 in the dark for 20 minutes at room temperature. After diluted in digestion buffer (aqueous 50 mM Tris, 1 mM DTT, pH 8.5), proteins were digested overnight at 37° C. with an enzyme/substrate (w/w) ratio of 1/25. FASP eluates were collected using digestion buffer and spiked with SIS peptides prior to peptide extraction using SOLAμ™ SPE 96 well Plates (Thermo, Cat. #60209-001).

Quantification of Peptides to Assess Protein Expression Levels. Quantification can be performed by liquid chromatography-multiple reaction monitoring mass spectrometry (LC-MRM) or other similar liquid chromatography tandem mass spectrometry approaches included parallel reaction monitoring (PRM) on high resolution instruments capable of accurate mass measurements. Preliminary data for this application was acquired using LC-MRM performed with a RSLCnano system interfaced with Altis triple quadrupole mass spectrometer (Thermo), but similar instruments could be used. The following details of the method can also be generalized to other instruments purchased from different vendors. Peptides are loaded on a trap column (Acclaim C18 PepMap 100, 100 μm ID×2 cm in length, 5 μm particle size, 100 Å pore size) using mobile phase A (aqueous 2% acetonitrile with 0.1% formic acid prior to separation at 40° C. on an analytical column (Acclaim C18 PepMap 100, 75 mm ID×25 cm in length 5 μm particle size, 100 Å pore size). The LC gradient using solvent A and solvent B (aqueous 90% acetonitrile with 0.1% formic acid) is delivered at 300 nL/minute and consisted of a linear ramps from 2%-8% B in 1 minute, 8%-30% B over 56 minutes, 30%-50% B in 14 minutes, 50%-90% B in 30 seconds, washing at 90% B for 6.5 minutes, and re-equilibration at 2% B for 15 minutes. The nanoelectrospray interface is operated in the positive ion mode. Q1 (peptide selection) and Q3 (fragment selection) resolution values are set to 0.4 and 0.7, respectively, to minimize interference from other intact peptides and to maximize signal for the fragment ions. Collision energy values are optimized for each transition on the instrument where the assay will be run. Scheduled MRM transitions use a retention time window of 5 minutes around the expected elution time and dwell times of 10-20 microseconds per transition, acquiring sufficient points across each peak for quantitation. A minimum of three transitions were selected for each light and heavy peptide (six in total per peptide pair). LC-MRM data can be analyzed by Skyline (Maccoss Lab, University of Washington) or similar software platforms (e.g. Thermo QuanBrowser or FreeStyle).

Peaks are detected for “light” and “heavy” ion signals. The light peptides are the proteolytic peptides that represent the proteins of interest in the tumor tissue sample: the heavy peptides are the SIS spiked at known amounts to enable quantification. The peak area of the light is compared to the heavy: that ratio is multiplied by the amount of the spiked SIS peptide to provide the minimum amount of the protein that was quantifiable in the tissue. Individual measurements can be used to provide the level of a biomarker or the ratio between different measurements can be used as a biomarker. Furthermore, the expression levels of multiple proteins can be integrated into pathway signatures.

### 4. Example 3

High Concordance of Gene Amplification and Increased Expression with Comprehensive Profiling

### Introduction

Herein, the concordance between gene amplification and RNA expression is reported by comparing results from testing samples for gene amplification by NGS and RNA expression with a custom 204 gene RNA expression panel, RNA STEP (Salah Targeted Expression Panel).

Moffitt Cancer Center has multiple and changing clinical trials for patients with cancer, many with biomarker-based inclusion and exclusion criteria. Feedback from Moffitt clinicians was elicited about which genes are most needed for clinical trial screening and designed a custom gene panel of 204 test and 12 housekeeping genes. RNA expression was performed with the NanoString NCounter platform, an amplification-free, multiplexed RNA profiling technology that is optimized for mRNA extracted from FFPE samples.

The RNA STEP results provide complementary information to NGS testing and was performed with remnant mRNA from NGS testing. The ability to perform RNA STEP with remnant RNA from other clinical testing helps preserve clinical tissue for other potential future needs. The addition of gene expression information to the NGS results about tumors may better inform difficult decisions made by the oncologists and patients about which targeted therapies or clinical trials to consider. With RNA expression information, patients are more likely to meet trial inclusion criteria which are often based on increased expression of specific proteins. Potential implications of this additional RNA expression information include faster time to a precisely matched therapy or trial and lower risk for trial rejection due to not meeting inclusion requirements.

### Methods

The gene content (Table 1) in the Salah Targeted Expression Panel (STEP) was designed based on feedback from Moffitt clinicians regarding which genes are most needed for clinical trial prescreening. The RNA STEP assay uses the nanoString nCounter TagSet chemistry technology. RNA STEP simultaneously measures the level of expression of 204 target genes, 12 housekeeping genes used for signal normalization and quality control, and 6 positive controls and 6 negative controls in a single hybridization reaction. The probe sequences for the expression panel were custom designed by the NanoString bioinformatics group. The oligonucleotide probes that contain both tag- and target-specific sequences which bind each target RNA to a specific reporter tag and universal capture tag were synthesized by IDT DNA Technologies.

Two cohorts with prior results and available remnant RNA samples were used in this study. The first cohort had reported clinical NGS results from a CLIA validated Moffitt STAR NGS (Illumina TST170) platform. A total of 102 independent RNA samples with various diagnoses in 19 different tissue sites with a collection span of 11 years were selected for this study. The STAR NGS mRNA clinical samples were extracted from a variety of FFPE sample types, including resected specimens, core biopsies, pleural fluid, and fine needle aspiration (FNA) cell blocks. The mRNA was extracted and purified according to the Qiagen DNA and RNA AllPrep protocol using the automated Qiacube instrument for clinical tissue samples. The Moffitt STAR NGS assay requires FFPE tissue with at least 10% tumor cellularity, a minimum of 50 tumor cells per H&E slide evaluation, and tissue from 5-10 unstained slides with 7 μm thickness. Sample tumor cellularity and diagnoses were verified and reviewed by a certified pathologist. The second cohort was composed of 25 samples from patients diagnosed with squamous cell lung cancer. The mRNA samples were extracted from frozen tissues using the Qiagen DNA and RNA AllPrep protocol. These samples had previously reported RNASeq results.

Sample RNA amounts between 13.2-300 ng were used in the hybridization reaction. RNA concentration and quality were assessed using a nanodrop ND-1000 spectrophotometer. An internal Moffitt custom-built informatics SQL-based system. Heracles, was used to register samples and calculate RNA concentration. A master mix preparation was prepared using nanoString recommended guidelines and the hybridization reaction was performed at 67° C. with heated lid at 72° C. for 24 hours. A universal RNA control from pooled human normal tissues (BioChain Institute. Inc.) was included in each run serving as a reference sample to assess batch to batch variability and to normalize the signal from each gene. The purification step in the nCounter prep station was set to “high sensitivity” protocol to increase binding of all molecules to the cartridge. Input files for the digital analyzer were prepared in the Heracles platform and then transferred via ftp to the digital analyzer. Fields of view (FOV) were set at 280 for digital imaging. nCounter data were processed and normalized using nanoString nSolver 4.0 advanced analysis software. RNA and data QC metrics of samples were also assessed in nSolver. The normalized data were transferred to the Heracles platform and log 2 ratio of the genes relative to the universal RNA control were calculated. The data was filtered and reported as a list of genes with high and low expression levels, with log 2 ratios greater than or equal to 2 and less than or equal to −2, respectively.

### Results

The Moffitt STAR NGS (TST170) and RNA STEP assays detected changes in 91 overlapping genes, including MET exon14 skipping. Of the 59 genes with DNA-based amplification detection by STAR NGS, 38 genes were mutually covered by the nanoString assay. Genes with log 2 ratio greater than or equal to 2 in the clinical sample relative to the pooled normal RNA control were designated as highly expressed. For example, if the MET gene had a log 2 ratio equal to 2, the expression of the MET gene in the clinical sample is 4 times higher than expression of the MET gene in the control sample.

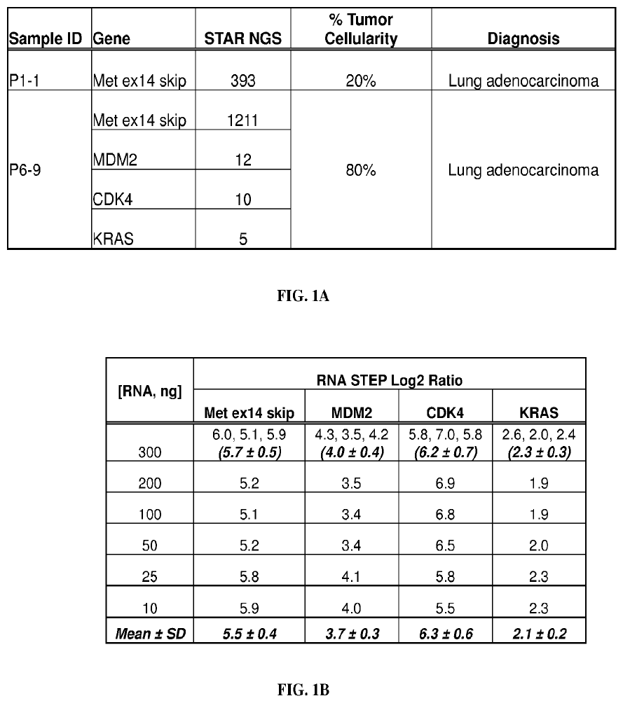

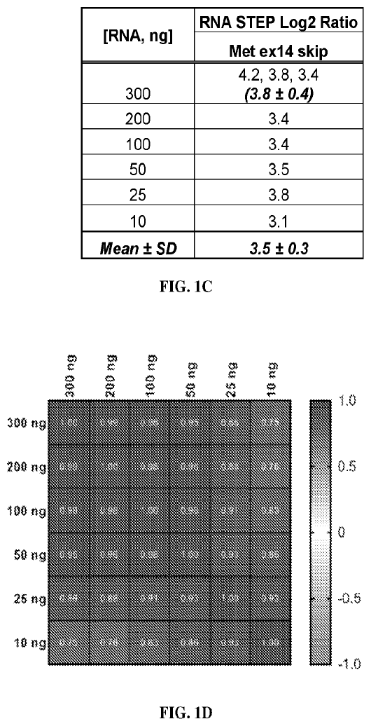

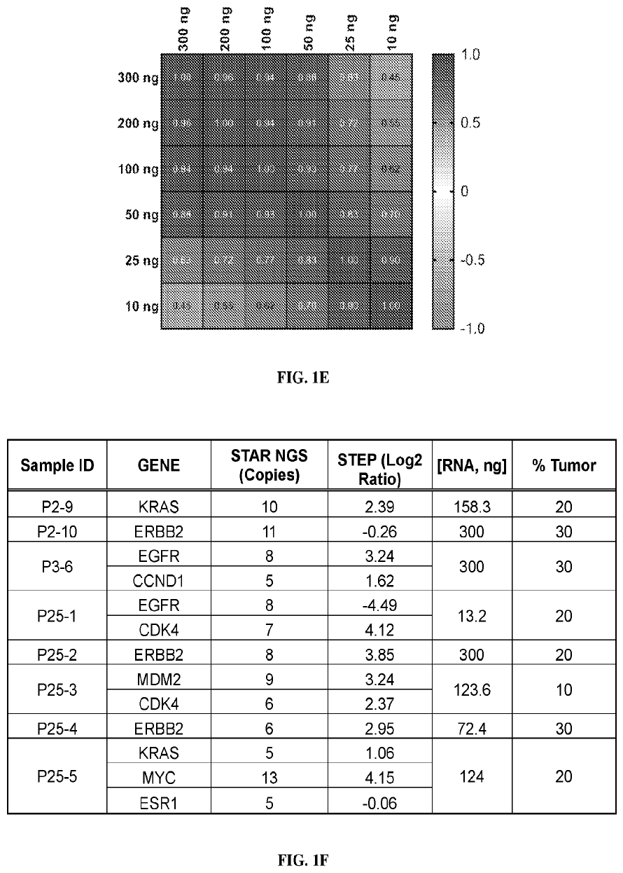

### Analytical Sensitivity/Limit of Detection (FIG. 1—all the Tables and Figures for LLLOD)

To investigate the analytical sensitivity (limit of detection) of the RNA STEP assay, two clinical RNA specimens representing MDM2, CDK4, and KRAS gene upregulation and MET exon 14 skipping were serially diluted with water to different concentrations (300, 200, 100, 25, and 10 ng). One sample (P1-1) had MET exon 14 skipping (supporting reads=393) and low tumor cellularity (20%). The other sample (P6-9) had 80% tumor cellularity, Met exon 14 skipping (supporting reads=1211), MDM2 (cn=12), CDK4 (cn=10), and KRAS (cn=5) amplification (FIG. 1A). The lower limit of detection (LLOD) was determined based on the sample passing quality control metrics during processing, and the ability to detect increased gene expression concordant with gene amplification or MET exon 14 skipping using the normalized log 2 ratio (log 2 ratio ≥2 for increased expression) relative to the pooled RNA control sample. At 10 ng, both samples failed data QC metrics. The sample with lower tumor cellularity failed data QC metrics at 25 ng as well. This suggests that 20% tumor cellularity and 10 ng may be close to the lower limit of detection.

Results (log 2 ratio per gene) from diluted samples were analyzed for concordance with undiluted samples. All results for the two clinical samples with 80% and 20% tumor cellularity diluted down to 10 ng were concordantly positive with the undiluted samples for MET exon 14 skipping, MDM2, and CDK4, and KRAS, except 2 results for KRAS at 200 ng and 100 ng had a log 2 ratio at 1.9, just below the positive cut-off of 2 (FIG. 1B,C). The log 2 ratio for KRAS with 300 ng input amount for 3 tests on different runs ranged from 2.0 to 2.6 (2.4±0.4). As also shown in FIG. 1B-C, the standard deviation within the same concentration (300 ng) and different concentrations is within 10%.

Pearson correlation was also performed to compare the results, log 2 ratios of the 204 genes from the 2 diluted samples. The correlation from 300 ng decreased as the concentration went down. At 10 ng, for sample with 80% and 20% tumor cellularity the pearson correlation was down to 0.75 and 0.45, respectively (FIGS. 1D and E).

In addition, 8 clinical samples with NGS gene amplification and inherently low tumor cellularity (10-30%) and/or RNA concentration (<150 ng, range 13.2 to 124 ng) to confirm that gene upregulation may be detected accurately in clinical samples with inherently lower tumor cellularity or RNA amount (FIG. 1F). With low tumor cellularity (N=8) and/or low RNA amount (N=4 with RNA <150 ng) in these samples, gene upregulation was detected in 8 of the 13 amplified genes (PPA=62.4%, log 2 ratio cut-off ≥2), lower than the PPA for clinical samples with >30% tumor cellularity (PPA=70.4% (90/146). With a lower cut-off of log 2 ratio ≥1, equivalent to 2-fold increase expression relative to the control sample, the PPA was higher at 76.9% (10 of 13). In the sample with lowest input of 13.2 ng of mRNA, NGS identified amplification in 2 genes. RNA STEP was concordantly positive for one gene (CDK4) and discordantly negative in the other (EGFR). Three clinical samples with low tumor cellularity and MET exon 14 skipping by NGS were analyzed (FIG. 1G). All 3 samples were concordantly positive for MET exon 14 skipping with RNA STEP with log 2 ratios above the positive cut-off of ≥2.

Given the results of the LLOD dilution experiments and additional experiments with clinical samples with inherently low tumor cellularity or RNA amount, the lower limit of detection was placed with inputs of higher than 10 ng and 10% tumor cellularity. Due to the lower PPA and lower tumor cellularity and/or RNA input amount (62.4%), a bolded qualifying statement was added to clinical reports from testing of samples with 530% tumor cellularity or ≤150 ng input amounts to explain the higher chance of false negative and false positive results.

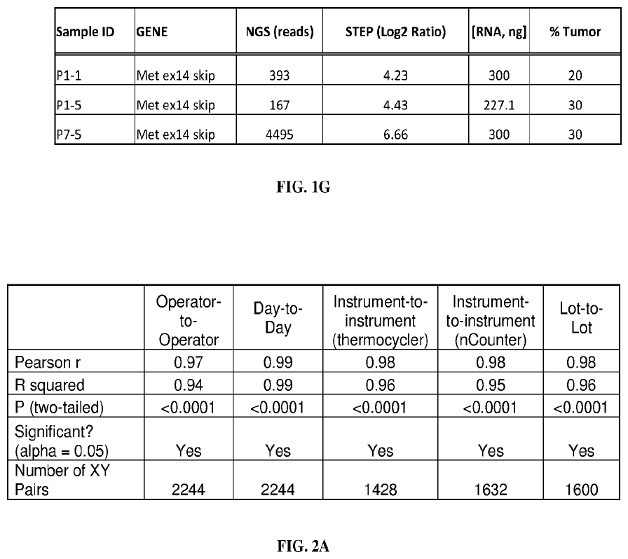

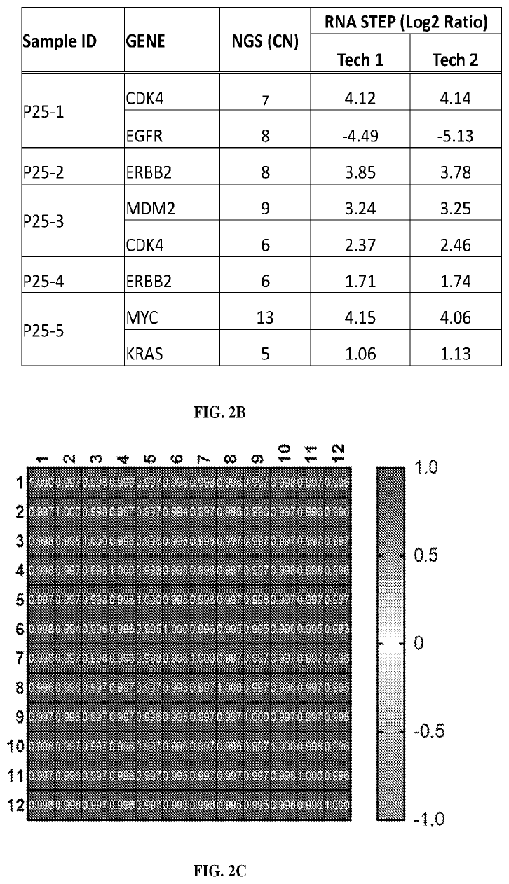

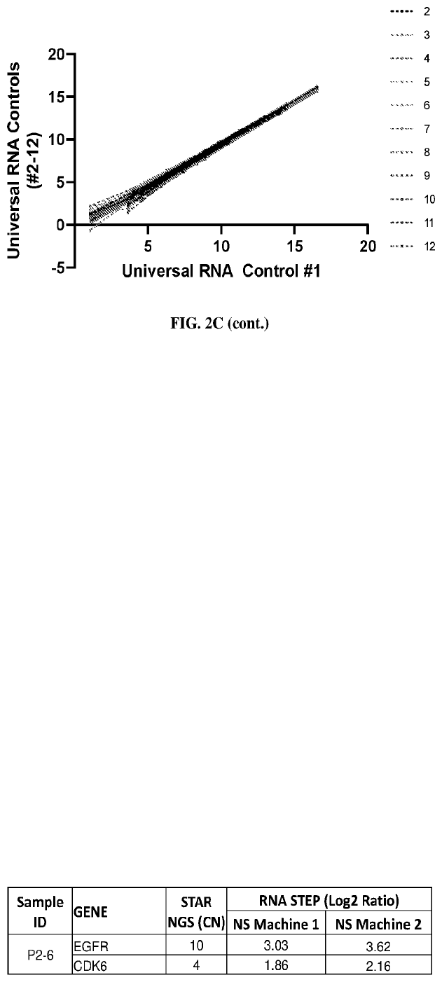

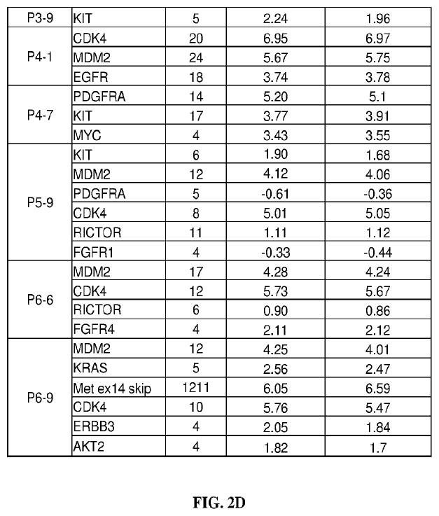

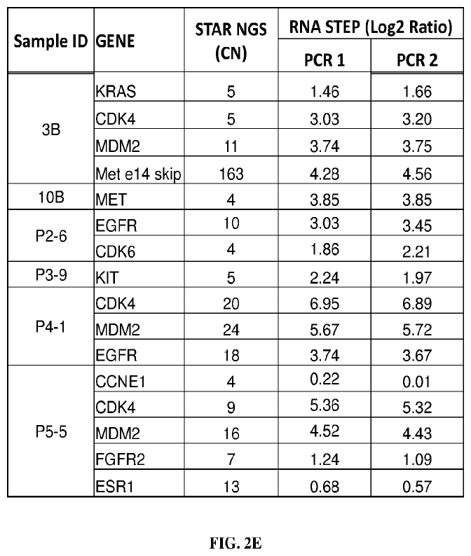

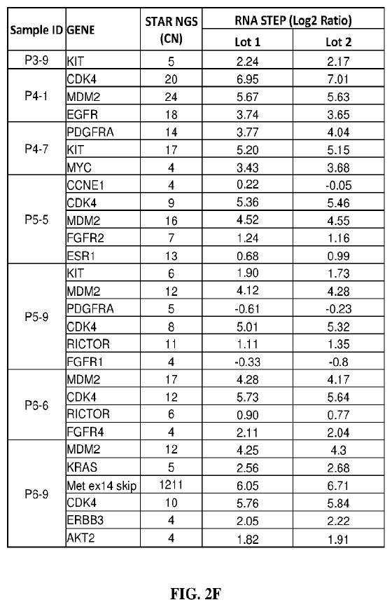

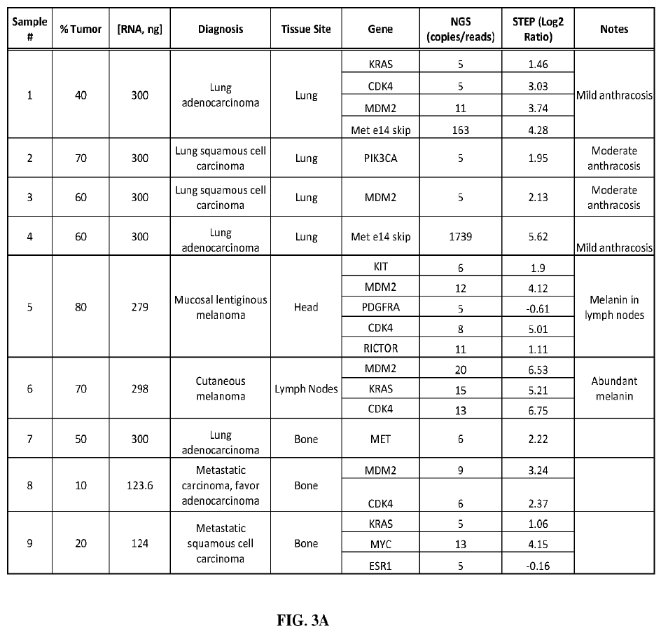

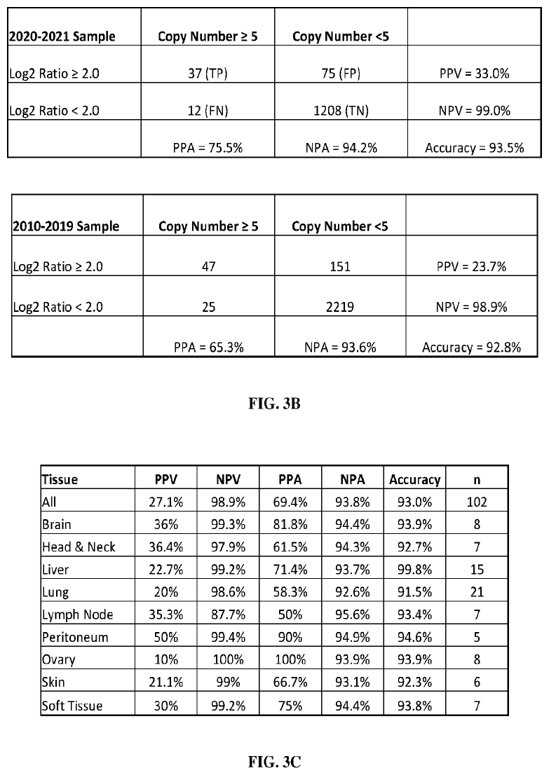

Precision. The precision of the RNA STEP assay was determined by multiple repeats of testing of samples within runs and between runs with comparison of results from different operators, days, instruments (thermal cyclers and nanoString nCounter), and reagent lot numbers. Data between replicates for all comparisons were analyzed with Pearson correlation. The results from all comparisons demonstrated excellent correlation (r>0.97, p<0.0001) (FIG. 2A). It was demonstrated that the RNA STEP assay returns similar results regardless of variations in testing conditions.

Analysis of operator-to-operator variability was performed by two technologists, who tested the same 11 diagnostic samples and 1 control in independent runs. Minimal operator-to-operator variability was detected with excellent correlation between gene log 2 ratio results for all duplicate samples (r=0.97, p<0.0001) (FIG. 2A). The RNA STEP log 2 ratio for genes with NGS paired results (7/8) are also highly concordant (FIG. 2B).

The pooled mRNA (Biochain) control specimen was included in triplicate within a single run to assess reproducibility and included in every run as a control to assess repeatability. The pearson correlation of the pooled mRNA control within 12 runs is >0.99 (FIG. 2C). A non-tumor lung sample was also repeated between runs to assess repeatability. 12 samples were repeated twice under the same conditions 14 days apart. Excellent correlation was observed between gene log 2 ratio results for the duplicate samples repeated 14 days apart (r=0.99, p<0.0001).

Two different runs were performed with 11 repeat samples and 1 control using different NanoString nCounter instruments and again using different thermocyclers instruments. The different nanoString instruments were located in the Moffitt Molecular Advanced Diagnostics (CLIA) and the Moffitt Molecular Research Core Laboratories. Excellent correlation was observed between samples run in two different nCounter machines (r=0.98, p<0.0001). Log 2 ratios of the genes with known NGS amplifications between runs are also close with each other. In addition, the samples with NGS amplifications also have good concordance (18125) with RNA STEP (cut-off, log 2 ratio=2) (FIG. 2D). The thermocyclers are both in the Moffitt CLIA laboratory. Excellent correlation was also observed with comparison of the thermocycler-to-thermocycler instrument results (r=0.98, p<0.0001). There is also a good concordance (11/16) in gene amplification between NGS and the RNA STEP with cut-off of log 2 ratio 2 (FIG. 2E).

A repeat run was performed with 11 clinical samples and 1 control with a different set of nanoString reagents (different lot number). With the change in lot number, 4 of the 204 gene probes were also changed to assess the ability to change probes. This additional step of changing 4 probes helps assure that as clinical needs for new biomarkers occur, probes may be changed with testing of duplicate samples before and after reagent or probe changes. Minimal lot-to-lot variability was detected with excellent correlation between gene log 2 ratio results for the duplicate samples (r=0.98, p<0.0001). Good concordance between NGS amplication and RNA STEP (19/28) was also observed using cut-off of log 2 ratio ≥2 (FIG. 2F).

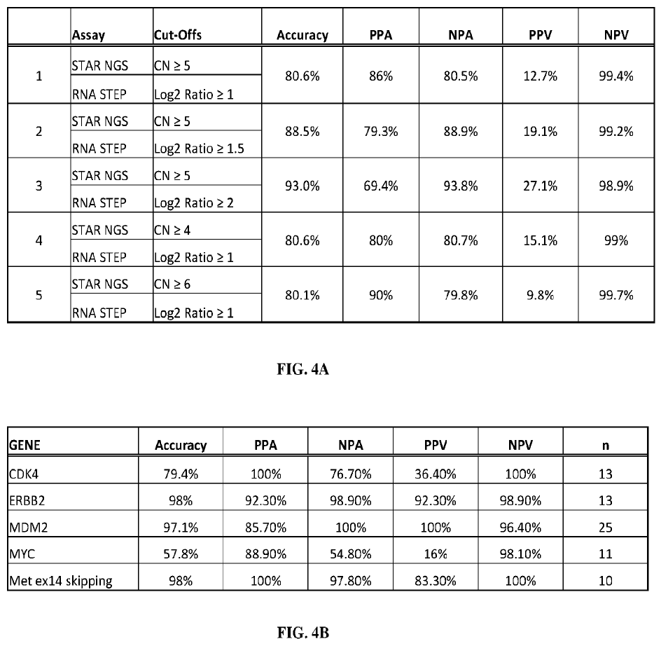

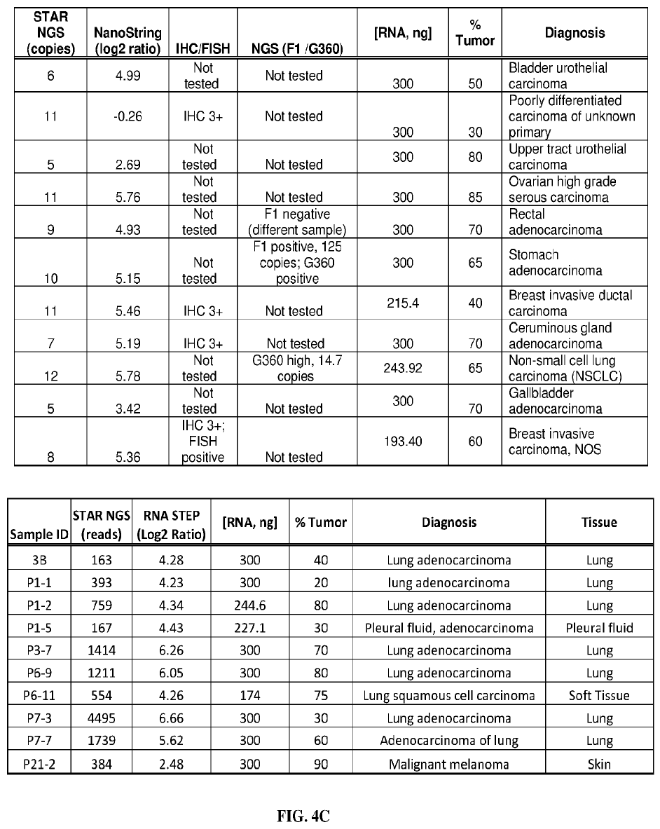

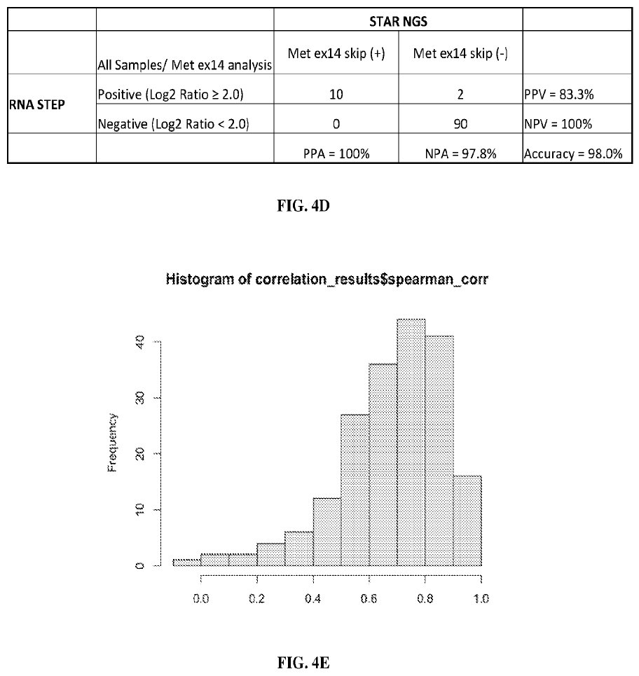

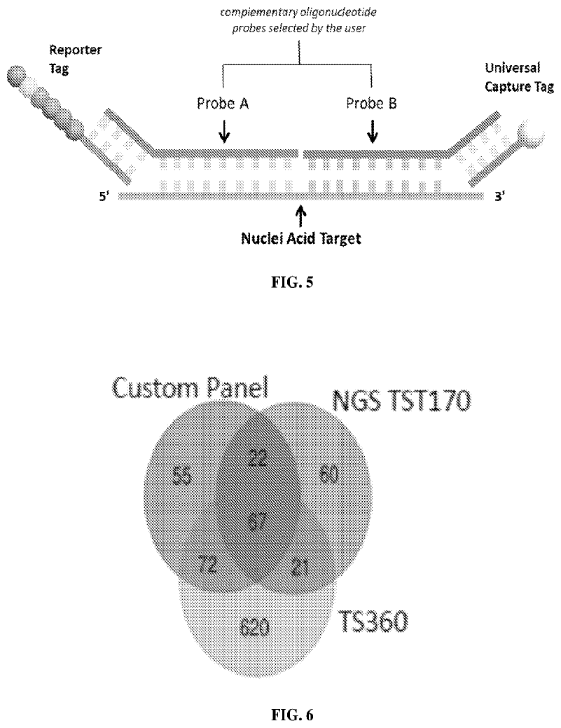

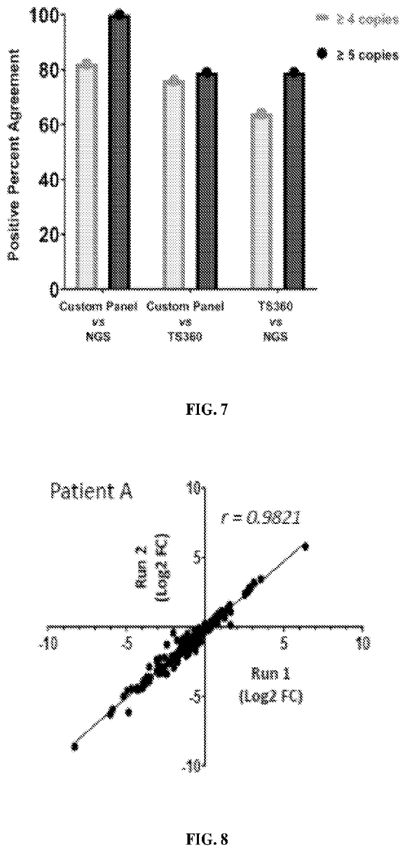

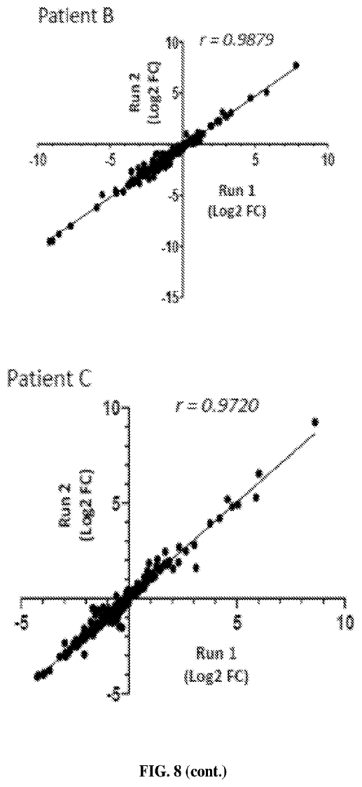

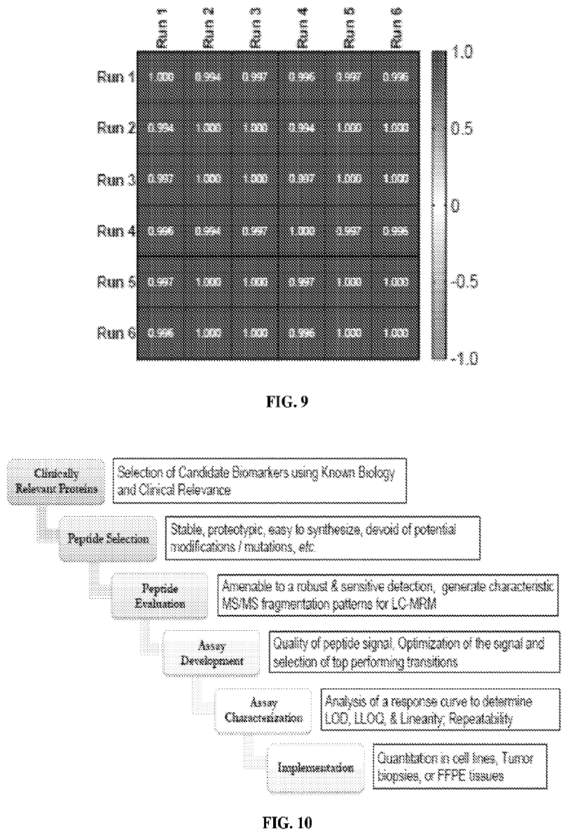

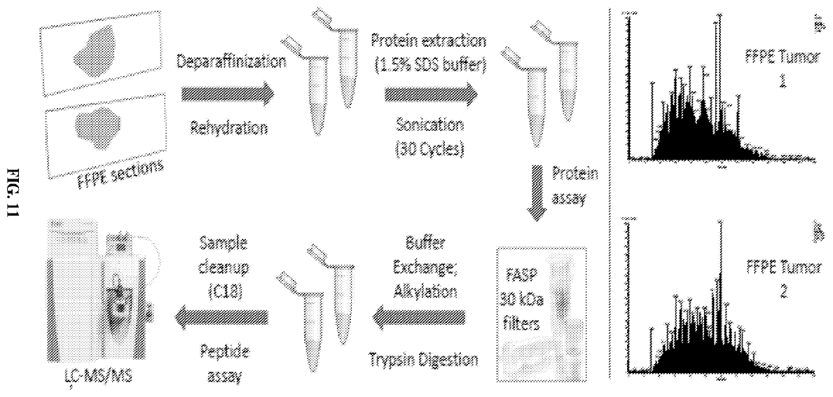

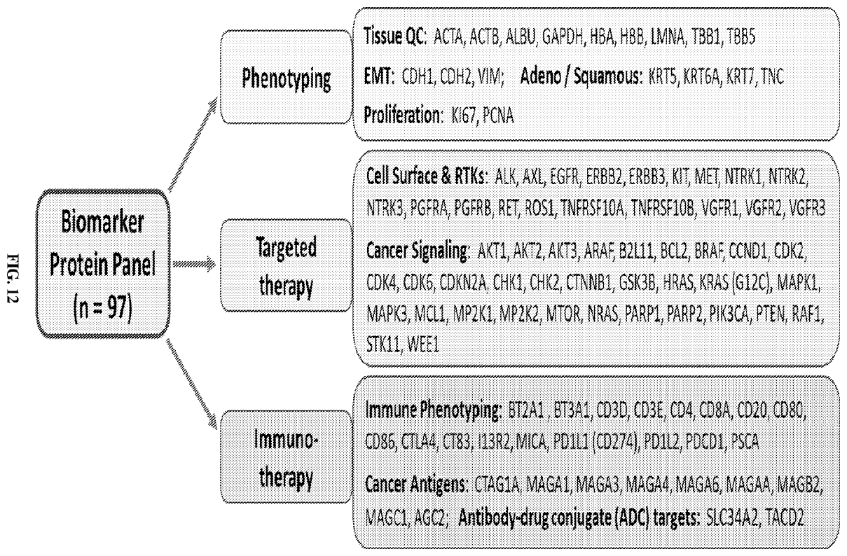

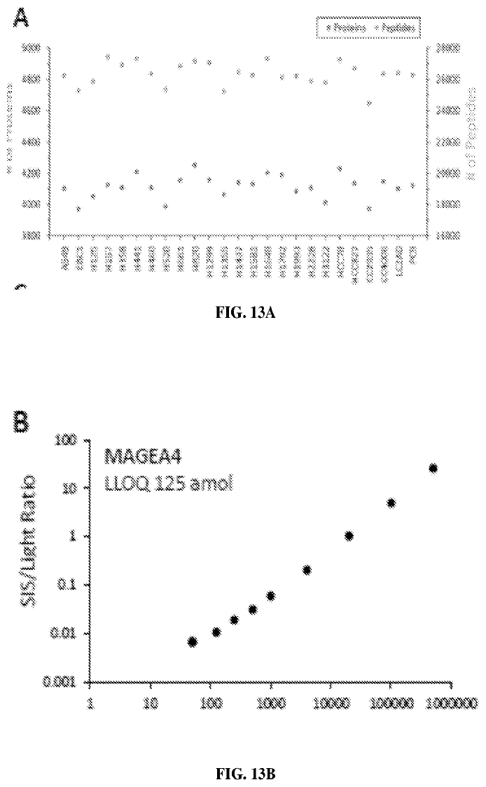

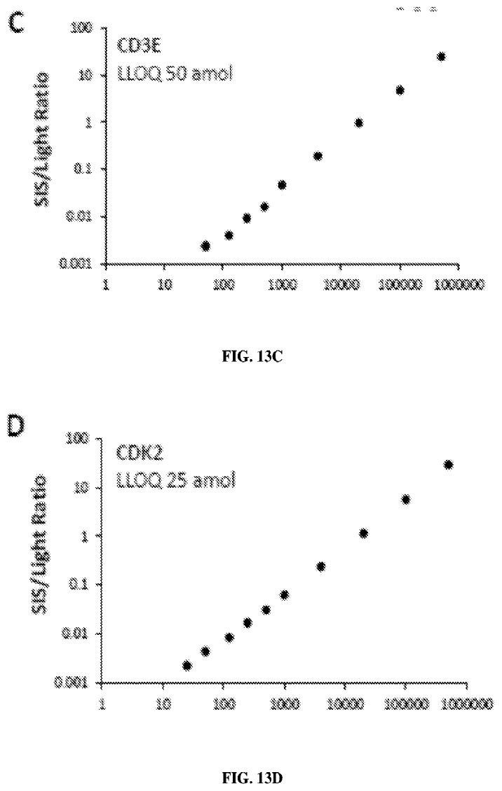

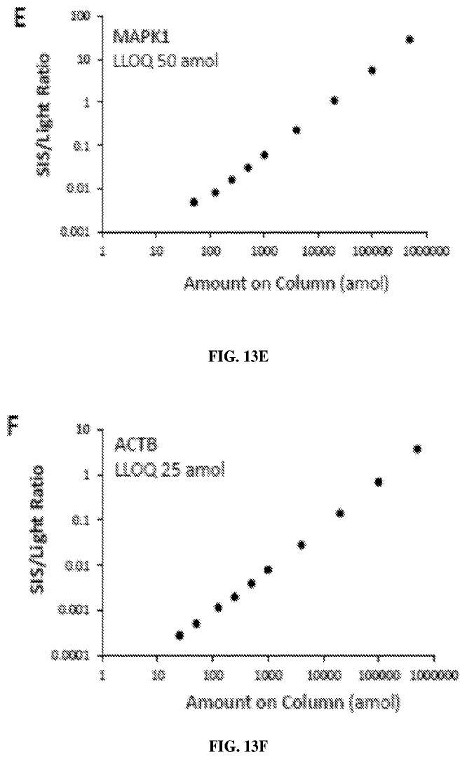

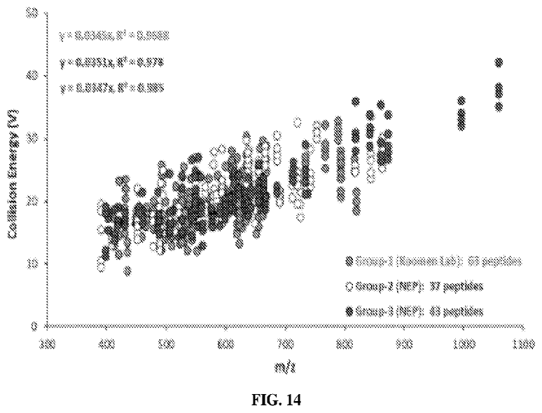

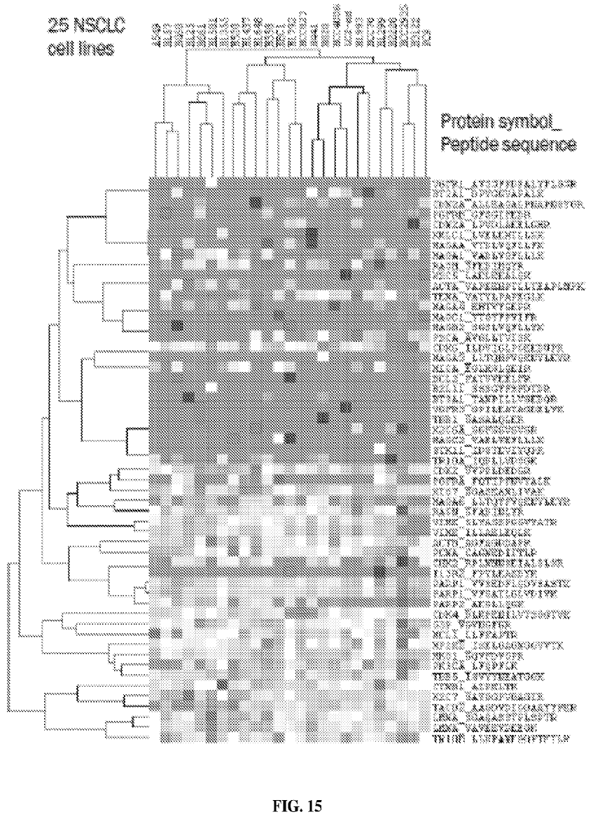

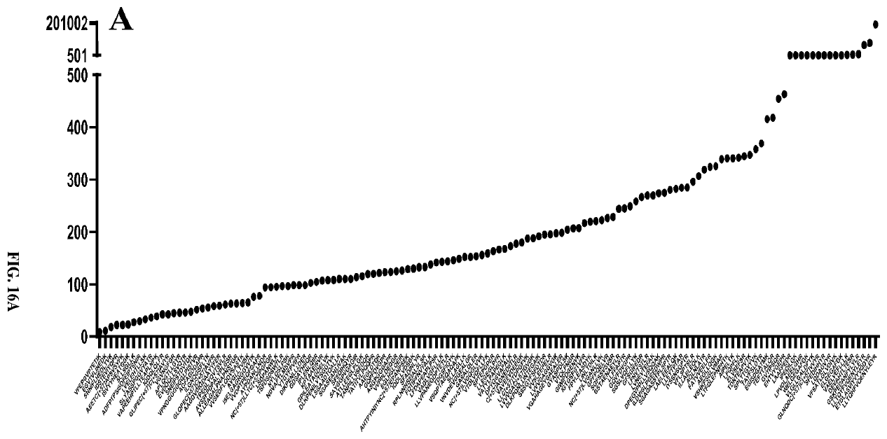

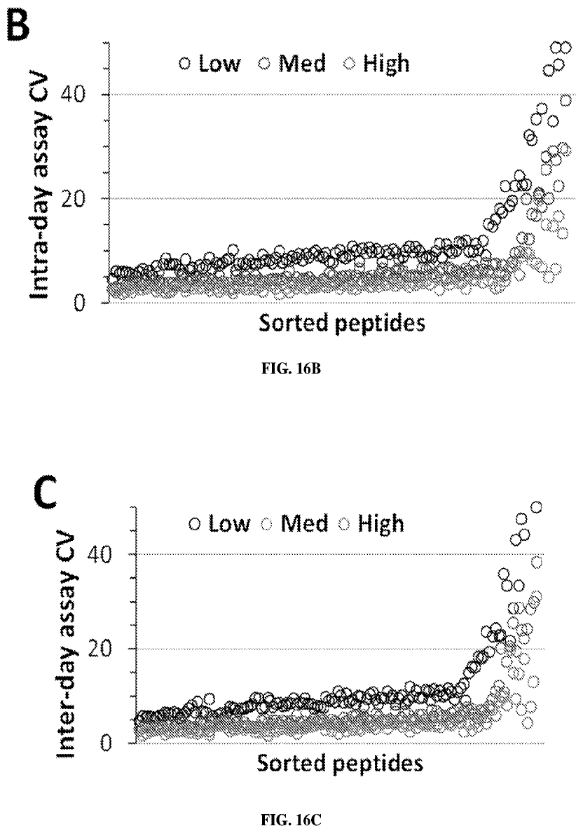

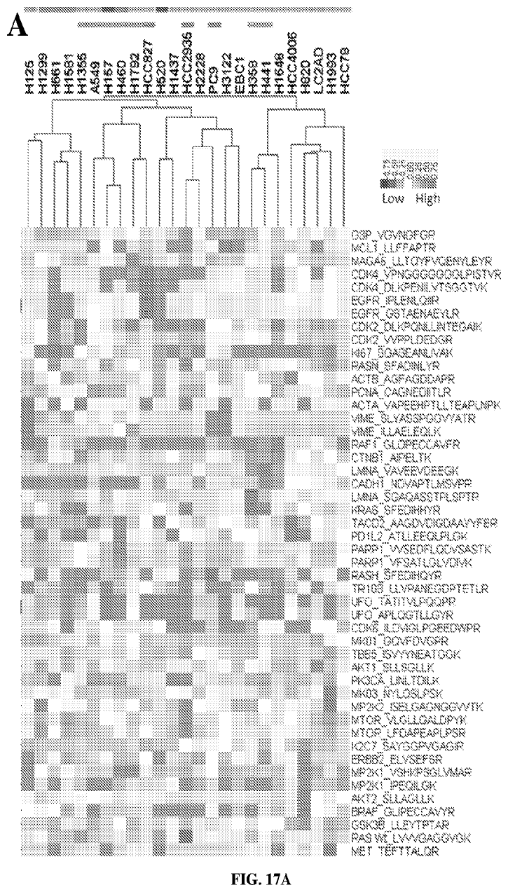

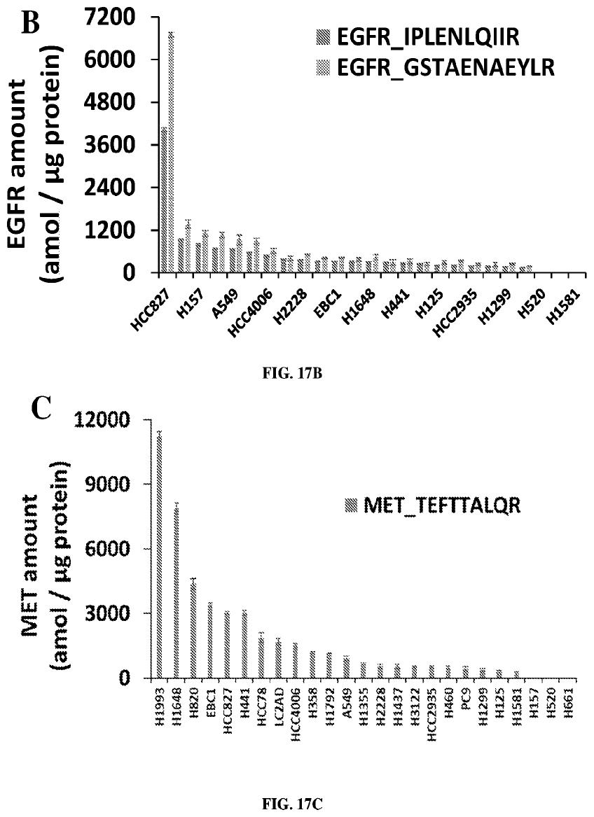

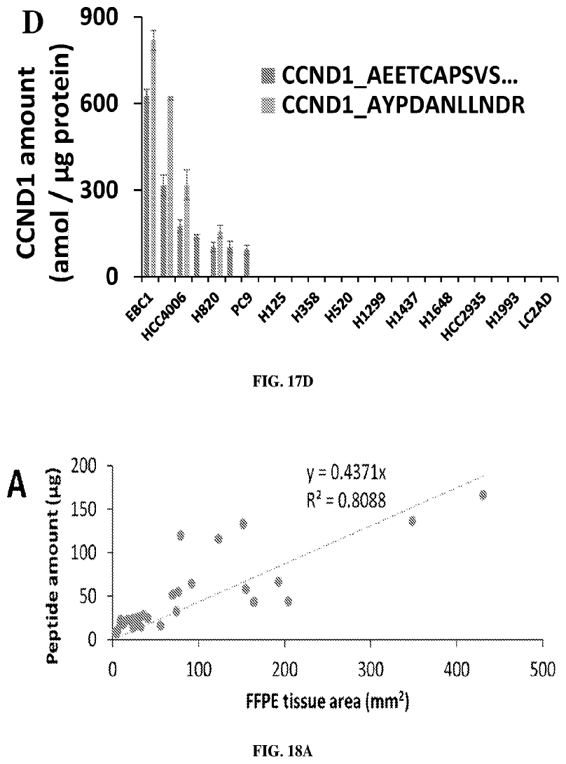

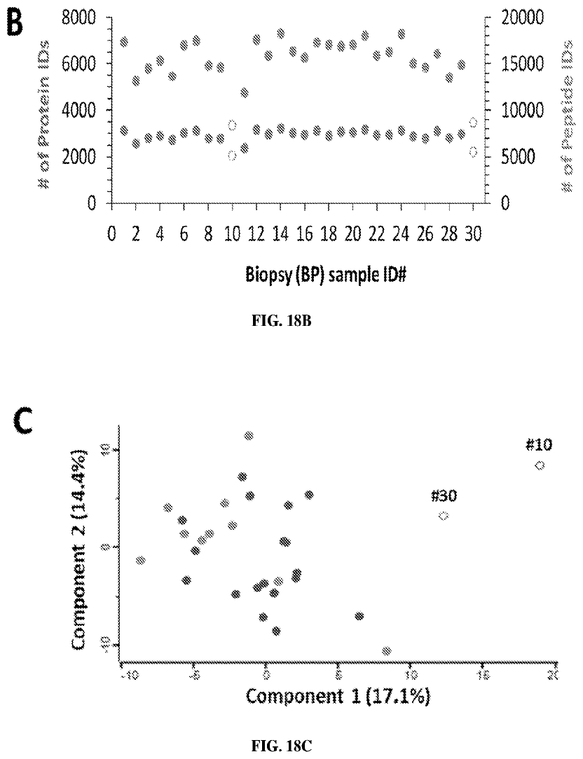

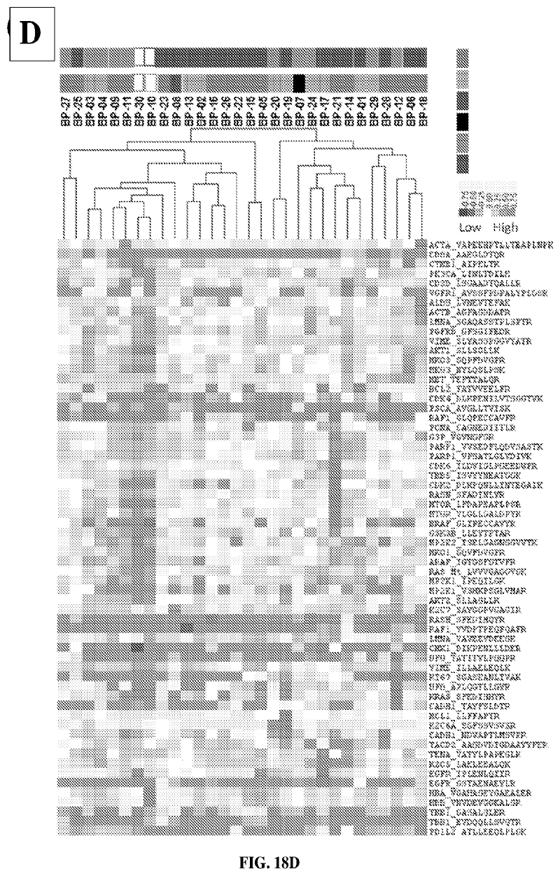

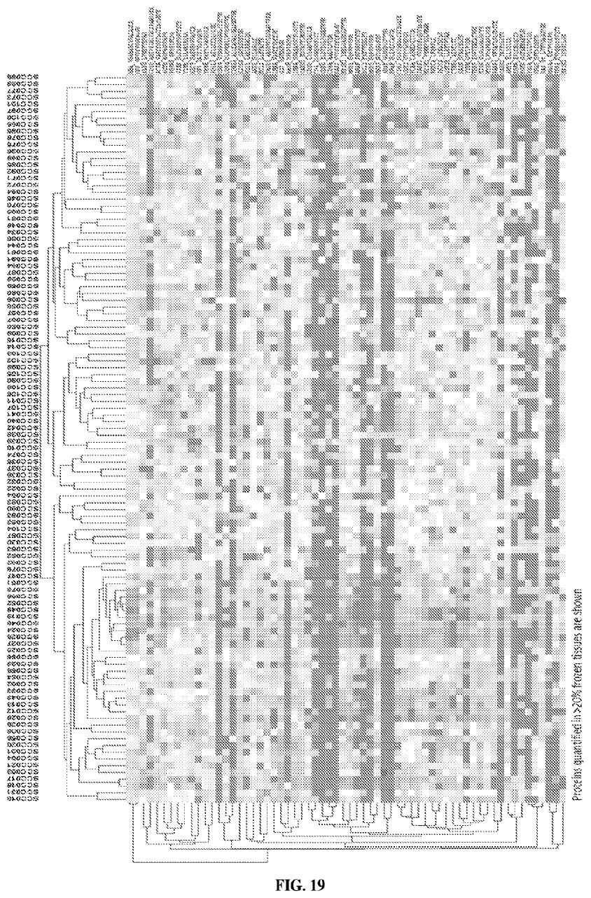

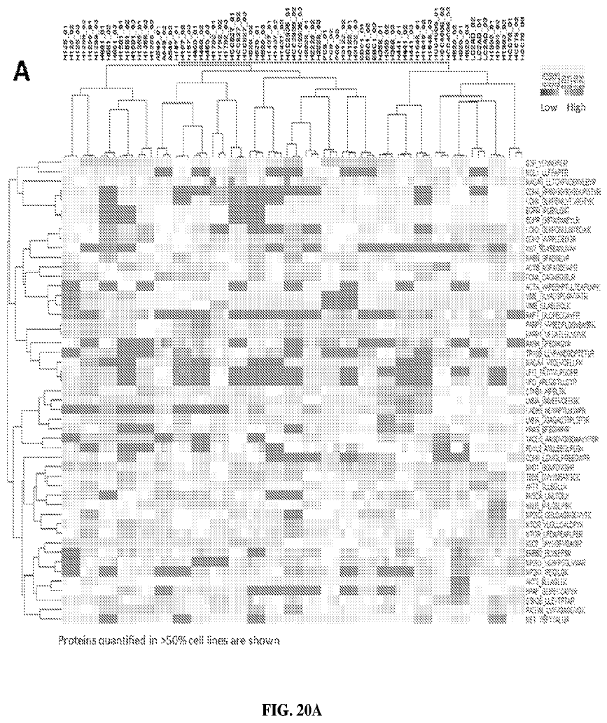

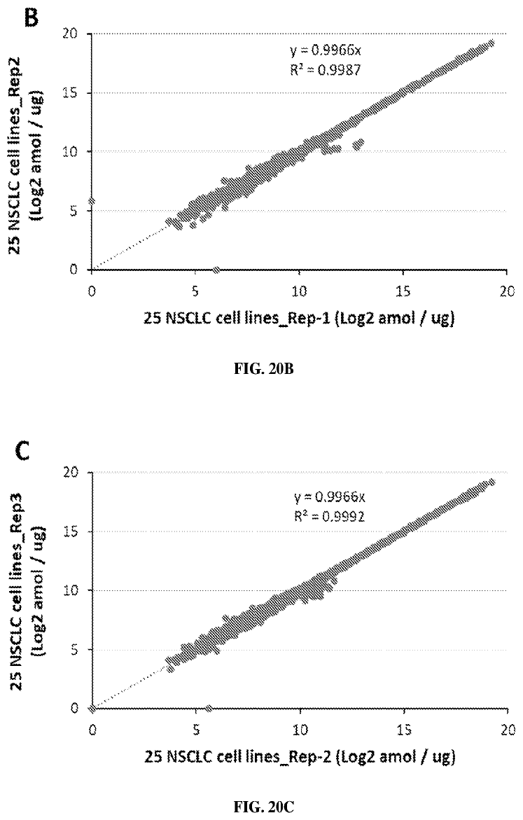

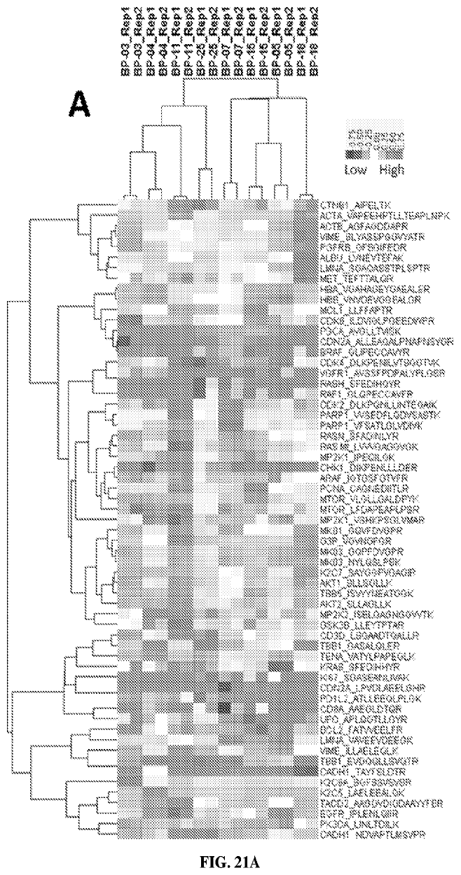

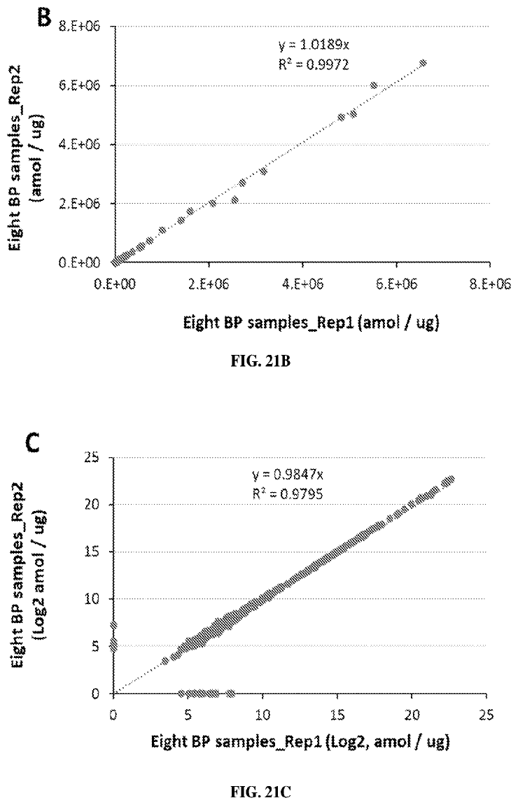

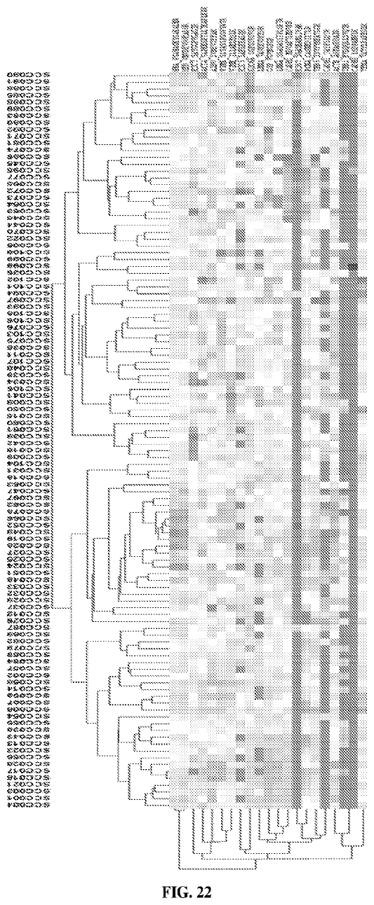

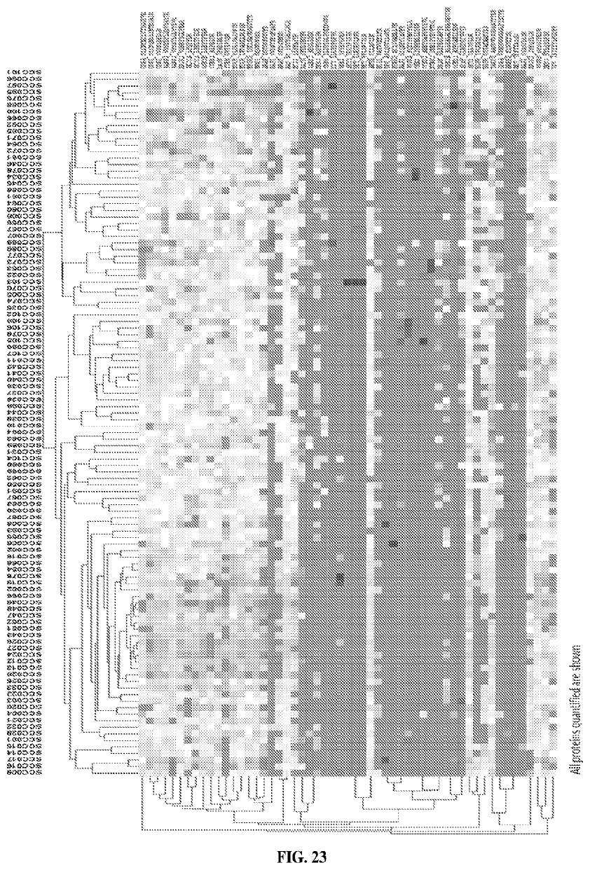

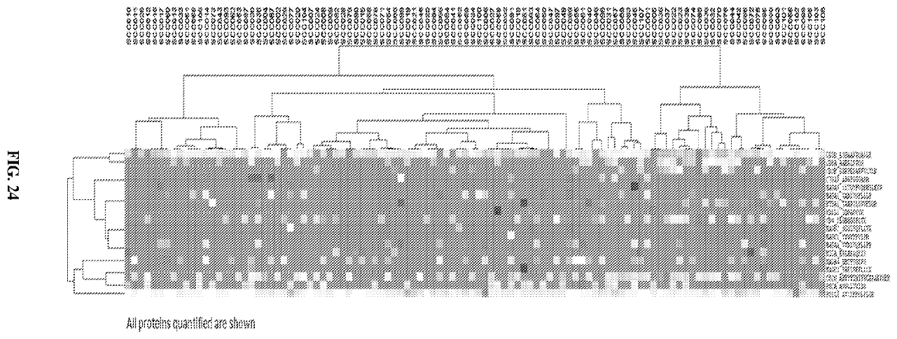

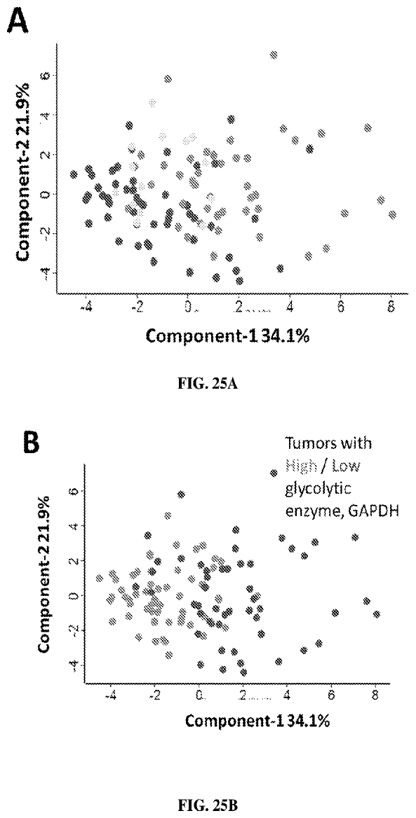

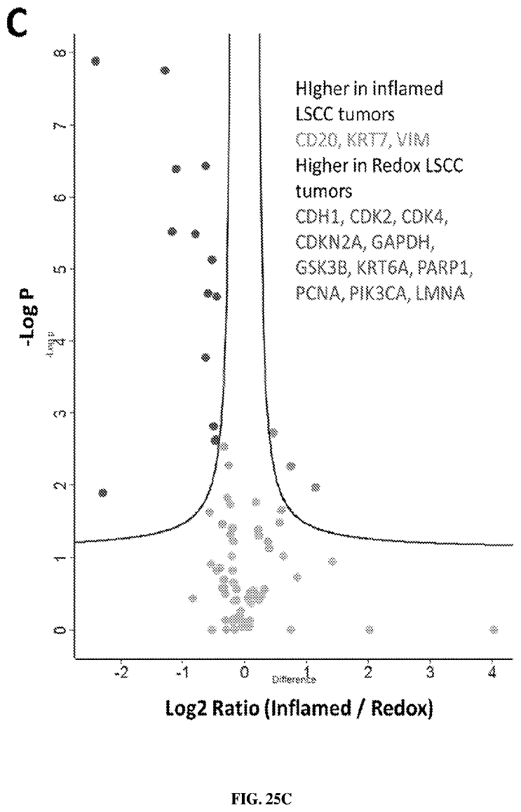

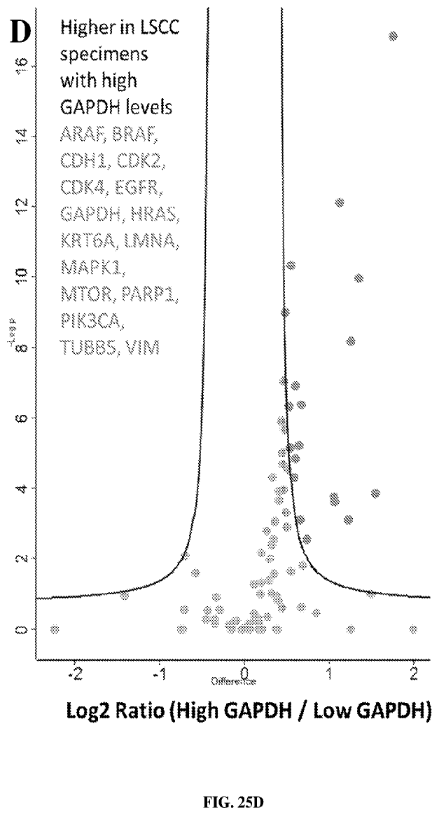

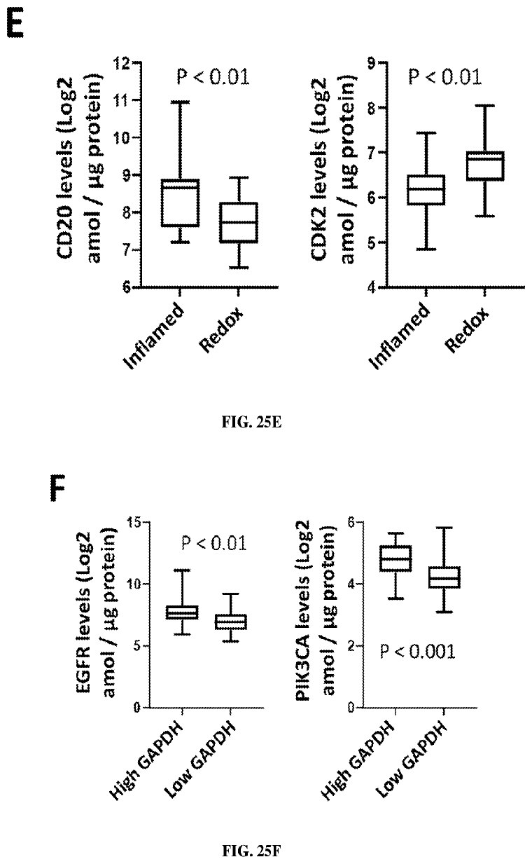

Specificity (Interfering substances). Sample conditions may vary, and this affects the accuracy of an assay. Specimens with potential interfering substances were included in testing. The specimens included 4 lung cancer samples with anthracosis (black pigment in lung caused by pollutant such as smoke), 2 melanoma samples with melanin, and 3 bone samples (FIG. 4A). The two lung cancer samples that had MET exon14 skipping detected by NGS were also positive for MET exon 14 by RNA STEP. In the lung cancer samples, gene upregulation (cut-off log 2 ratio ≥2) was detected in 3 of 5 genes with increased copy number by STAR NGS (cut-off ≥5 copies). The two discordant results both had 5 copies near the cut-off for calling gene amplification. With a lower log 2 ratio cut-off of 1 gene upregulation, all 5 genes would be called positive. In the melanoma samples, gene upregulation (cut-off 5 copies), a lower log 2 ratio of 1 for gene upregulation would have a PPA of 86% (7 of 8)

The results from samples collected within the past two years, 2020-2021 versus older samples, 2010-2019 were compared. The PPA was higher in the newer samples, 75.5% versus 65.3% even though the accuracy of samples collected in the past 2 years (2020-2021) versus older samples (2010-2019) was similar, 93.5% versus 92.8%, respectively. FIG. 4B shows the comparison of PPA, NPA, PPV, NPV and accuracy by sample age.

The effect of background tissue type on the accuracy of RNA STEP versus STAR NGS results were also evaluated. The sample set included 21 lung, 15 liver, 8 brain, 8 ovary, 7 head and neck, 7 lymph node, 7 soft tissue, 6 skin, 5 peritoneum samples. The remaining tissue sites had 3 or fewer representative samples: pleural fluid (3), urinary bladder (3), bone (3), rectum (2), breast (2), colon (1), kidney (1), pancreas (1), prostate (1), and stomach (1). Accuracy was >93% for all tissue sites (FIG. 4C) using ≥5 copies as the positivity cut-off for gene amplification by STAR NGS and a log 2 ratio ≥2 as the positivity cut-off for gene upregulation by RNA STEP.

Taken together these results demonstrate that lung and melanoma samples may be accepted even with abundant anthracosis and melanin. Samples processed with decalcification or freezing are also acceptable. Older samples may be tested up to 10 years old, but if older than 2 years should be reported with qualification that there may be a higher risk for false negative results.

Diagnostic validation/concordance study. RNA STEP results from the clinical samples were compared with STAR NGS (CLIA) results for accuracy, positive percentage agreement (PPA), negative percent agreement (NPA), positive predictive value (PPV), and negative predictive value (NPV). Different log 2 ratio cut-offs (1,2, and 3) for RNA STEP detection of MET exon 14 skipping in samples with MET exon 14 skipping reported by STAR NGS were specifically compared. RNA STEP accuracy, PPA, NPA, PPV, and NPV were evaluated for detection of gene upregulation in clinical samples with gene amplification identified by STAR NGS using cut-offs of 1, 1.5, and 2 for RNA STEP and cut-offs of >4, >5 and >6 copies for NGS gene amplification.

Comparison for results for gene amplification (cut-off: 5 copies) versus upregulation (cut-off: log 2 ration ≥2) of the same gene in patient samples demonstrated an overall accuracy of 93%, despite biological differences between gene amplification and upregulation and that gene amplification is DNA-based detection. Accuracy was 93.0% (positive percentage agreement=69.4%; negative percentage agreement=93.8%; positive predictive value=27.1%: negative predictive value=98.9%) for all comparable gene amplification versus expression results. FIG. 4A shows the accuracy, PPA, NPA, PPV, and NPV for different STAR NGS and RNA STEP cut-offs. Accuracy was higher at 97.1% for MDM2 gene results. Of 25 samples with MDM2 gene amplification, 18 had highly upregulated gene expression (log 2 ratio % 2). Only 3 samples with negative MDM2 amplification in STAR NGS had positive RNA STEP result (log 2 ratio >2), 97% or 99 samples were negative for both MDM2 gene amplification and gene upregulation in both assays.

Higher PPA was observed for specific genes such as CDK4 (100%, N=13), ERBB2 (92.3%, N=13), MDM2 (85.7%, N=20), and MYC (88.9%, N=11) (FIG. 4B). For the 13 ERBB2 cases, 7 had additional ERBB2 results by IHC, FISH, Foundation 1 solid tumor NOS, and/or Guardant 360 cfDNA NGS (FIG. 4C). Of note, the case with a discordant result for STAR NGS versus RNA STEP was positive by HER2/neu IHC and likely a false negative RNA STEP result. This sample had the lowest tumor cellularity (30%) of the 13 samples and was a lymph node metastasis. The negative result may be due to dilution of the tumor RNA by non-tumor cell RNA and helps justify including a qualifying statement on the report for samples with lower tumor cellularity to explain the higher chance for false negative results.

The general negative predictive value (NPV=98.9%) and negative percentage agreement (NPA=93.8%) were high, meaning that in general non-amplified genes did not have high gene expression and furthermore that if gene expression was not upregulated, gene amplification was unlikely. The NPV for specific genes are CDK4 (100%), ERBB2 (98.9%), MDM2 (96.4%), and MYC (98.1%). The NPA are high for MDM2 (100%), ERBB2 (98.9%) and lower for CDK4 (76.8%), and MYC (54.8%). Upregulated gene expression generally did not predict gene amplification well as reflected by a low general positive predictive value (PPV) of 27.1%. An explanation for the overall low PPV might be that there are many causes for gene upregulation other than gene amplification. A caveat is that upregulation of a few specific genes, such as EGFR, ERBB2, and MDM2, better predict amplification (100%, 92.3%, and 100%, respectively). Gene amplification and upregulation had moderate positive percentage agreement (PPA) at 69.4% with higher PPA for specific genes such as CDK4 (100%), ERBB2 (92.3%), MDM2 (85.7%), and MYC (88.9%).

Specific comparison of results of RNA STEP and STAR NGS for MET exon 14 skipping was performed for 102 cases (FIGS. 4C and D). STAR NGS was defined as MET exon 14 skipping positive if MET exon 14 skipping was present in the clinical STAR NGS report. Of the 102 STAR NGS reports reviewed, 10 were positive for MET exon 14 skipping. All 10 were concordantly positive for RNA STEP with a positive cut-off of log 2 ratio ≥2. The 10 positive samples had an average RNA STEP log 2 ratio of 4.9, ranging from 2.5 to 6.7. Two samples were positive for Met exon 14 skipping by RNA STEP but not in the STAR NOS clinical report. Both samples were lung adenocarcinomas with MET amplification. They had MET exon 14 skipping log 2 ratios of 2.1 and 2.8. The sample with the log 2 ratio of 2.8 also had an EGFR exon 19 deletion by STAR NGS. One possible explanation for the discordant STAR NGS (negative) and RNA STEP (positive) results is that this patient may have been beginning to develop evolutionary resistance to EGFR targeted therapy with an increase in MET exon 14 skipping, but still below the diagnostic threshold for STAR NOS. All other 90 samples were concordantly negative for MET exon 14 skipping by both RNA STEP and STAR NGS. Accuracy with a log 2 ratio positive cut-off of 22 was 98.0%. Orthogonal testing is recommended for MET exon 14 skipping detected with log 2 ratio between 2 and 4 prior to clinical action.

Results of RNA STEP and RNAseq were both performed on the same mRNA from 25 frozen lung squamous cell carcinomas. The results from the 25 samples for 191 genes covered by both assays were compared by correlation analysis. Histogram of the correlation between the RNA STEP and the RNASeq is shown in FIG. 4E. Overall, the RNA STEP and RNAseq data were well correlated with a mean correlation of 0.68. Of the 191 genes analyzed, 92.7% (177/191) had a correlation p-value<0.05.

### Discussion

The initial performance of the Moffitt Custom RNA Expression Panel exhibited high concordance with NGS amplification results and good reproducibility with repeated samples. Remnant RNA from clinical genetic testing can be tested with the Custom RNA Expression Panel to provide innovative clinical information about patients' cancers and CLIA validation may facilitate clinical trial prescreening.

Accuracy between gene amplification results detected by DNA-based NGS versus gene upregulation detected by RNA-based RNA expression profiling was higher than expected (92.9%). The relationship between gene amplification and upregulation varied by specific gene. Overall if a gene was not upregulated, amplification of that gene was unlikely (NPV=98.9%) and if a gene was not amplified, it also was usually not upregulated (NPA=93.7%). Specific genes, such as CDK4, ERBB2, MDM2, and MYC, had higher PPA (all >=85%) than others (general PPA=70.4%). Upregulated gene expression generally did not predict gene amplification well as reflected by a low general positive predictive value (PPV) of 27.9%. The explanation for the low PPV might be that there are many causes for gene upregulation other than gene amplification.

Gene upregulation can be detected with reasonable accuracy (92.9%) even though gene upregulation was a poor predictor of gene amplification (PPV=27.9%). These results are consistent with knowledge that many factors can upregulate genes other than amplification of that gene. Importantly, absence of gene upregulation predicts lack of gene amplification (NPV=98.9%). Genes that were amplified were usually also upregulated (PPA=70.4%), with higher PPA (≥85%) for CDK4, ERBB2, MDM2, and MYC. Since gene upregulation can lead to increased protein expression even in the absence of gene amplification, detection of gene upregulation may provide meaningful information beyond the gene amplification.

### Conclusion

Accuracy between gene amplification results detected by DNA-based NGS versus gene upregulation detected by RNA-based RNA expression profiling was higher than expected (93%). The relationship between gene amplification and upregulation varied by specific gene. Overall if a gene was not upregulated, amplification of that gene was unlikely (NPV=98.9%) and if a gene was not amplified, it also was not upregulated (NPA=93.8%). Specific genes, such as CDK4, ERBB2, MDM2, and MYC, had higher PPA (all >85%) than others (general PPA=69.4%). Upregulated gene expression generally did not predict gene amplification well as reflected by a low general positive predictive value (PPV) of 27.1%. The explanation for the low PPV is that there are many causes for gene upregulation other than gene amplification.

### 5. Example 4

### Custom-Designed RNA Salah Targeted Expression Panel (STEP) Using the NanoString Platform Principle

NanoString is an amplification-free multiplexed RNA expression profiling technology that is optimized for mRNA extracted from FFPE samples. The nCounter Elements technology (FIG. 5) which is used in this assay is based on direct digital molecular counting of target RNA through the use of an nCounter Elements TagSet and target-specific oligonucleotide probe pairs (Probes A and B) designed for each RNA of interest by the user. The Elements TagSet consists of Reporter and Capture Tags. The Reporter Tag is a fluorescence color-coded probe, which carries the unique pattern of six spots of color on its 5′ end, creating fluorescent barcodes that can be individually resolved and counted during data collection. The Universal Capture Tag carries biotin moiety on the 3 end which enables hybridized complexes to be captured on the imaging surface.

The three main steps involved in the NanoString nCounter Flex Analysis System are: (1) Hybridization using the thermal cycler. During hybridization (FIG. 5). Probe A hybridizes to a specific Reporter Tag and the 5′ region of the target RNA sequence. Probe B hybridizes to the Universal biotinylated Capture Tag and the 3′ region of the target RNA sequence. The structure formed after hybridization is called a Tag Complex. In this step, the Elements TagSet and oligonucleotide Probes A & B are placed into a reaction in massive excess relative to the RNA sample to ensure each target undergoes hybridization; (2) Purification of the Tag Complex in the nCounter Prep Station: After hybridization, excess Probes and Tags as well as non-target nucleic acids are washed away in the nCounter Prep Station. The purification process uses nCounter Prep Plates containing reagents necessary for post-hybridization processing and immobilization onto the nCounter Cartridge; and (3) Barcode Digital Imaging and Counting in the nCounter Digital Analyzer: RNA molecule is counted individually based on the color-coded Reporter Tag detected in the NanoString nCounter Digital Analyzer.

The Salah Targeted Expression Panel (STEP) was designed based on feedback from Moffitt clinicians regarding which genes are most needed for clinical trial prescreening. The gene content in the RNA STEP custom panel is shown in Table 1. Positive and negative controls are also included in the Elements TagSet. The NanoString Custom Panel simultaneously measures the level of expression of 204 target genes, 12 housekeeping genes used for signal normalization, 6 positive controls, and 6 negative controls in a single hybridization reaction. A universal RNA control from pooled human normal tissues is also included in each run serving as a Reference sample to assess batch to batch variability and to normalize the signal from each gene.

The informatics system, Heracles, is used to register samples, calculate RNA concentration, generate log 2 ratio results, and create reports. It is a custom internal SQL-based database developed by Dr. Pedro Cano.

### Sample Registration

Each sample has a ‘sample name’ given for its identification in the interface with the Heracles app and nSolver. The ‘sample name’ is based on the MRN and the sample PK. For instance, ‘00823627_134’ is the ‘sample name’ for sample with MRN 823627 and corresponding PK 134. NanoString and NGS share the same subject and sample tables database.
1. Double click the Heracles shortcut in the desktop.
2. Heracles screen pops up and click on the “Registration” tab.
3. Registration screen pops up.
4. Create Subject Record
   a. For samples with MRN: enter the MRN on the MRN field and press green “search” button. All fields associated with that particular sample will be populated. Delete data for old sample and enter data for new sample. Press yellow “create sample record” button.
   b. For samples without MRN: Enter “0” in the MRN field, enter given name and family name and then press yellow “create subject record” button. An identifier will be created (i.e, pk5911). Then fill in the remaining fields. Enter ‘x’ if no information is available and then press beige “create sample record” button.

### Creating a Run

A maximum of 12 samples can be run in one experiment. In each run, one well is reserved for the universal RNA as a control.
1. Double click on the Heracles shortcut in the desktop.
2. On the main menu, go to the “Testing” tab and scroll down to “Run Setup”.
3. The “Run Setup” screen pops up.
4. Enter MRN and click gray “subject and samples” button. The samples for the subject will appear on upper-right grid.
5. Select sample and click on purple “select sample” button. A record for the setup will be created and appear in lower-right grid.
6. Continue adding samples for the run.
7. Enter lot number.
8. After the samples have been added, press yellow “create run” button.
9. The run number will appear in the lower-right corner.

### RNA Concentration and Quality

The recommended NanoString RNA concentration and purity for FFPE samples is shown in Table 2. The ideal input amount as recommended by Nanostring is 150-300 ng. The minimum input amount established by the validation is 10 ng.

### Reagents & Materials

| Item | Storage | Expiration |
|---|---|---|
| Ultrapure DNase/RNase-free | RT | 1 Year |
| distilled water | | |
| 1.5 mL nonstick RNase-free | RT | None |
| microcentrifuge tubes | | |

### Equipment

Micropipette and tips: 2 μL
Mini centrifuge
Nanodrop™ ND-1000 Spectrophotometer

### Procedure

1. Thaw the RNA samples on ice.
2. Briefly spin down samples (<30 seconds) in the mini centrifuge.
3. Open ND-1000 V3.8.1 program using the computer connected to the Nanodrop.
4. Once the program is opened, click on the “Nucleic Acid” application module.
5. Open the Nanodrop arm and load a 2 μL Ultrapure DNase/RNase-free distilled water as blank sample and press “OK” to read the blank.
6. When the machine is finished, open the sampling arm and clean the blank off the upper and lower pedestals with Kimwipe.
7. In order to enter sample information in Nanodrop, open Heracles in the laboratory computer next to the Nanodrop instrument. In the Heracles main menu click on the “See Data” tab and select “run data”. In the run data window, click gray ‘see runs’ button to get a list of recent runs. Select desired run and click purple ‘run samples’ button.
8. Copy the ID numbers in the ‘FK_Sample’ colunm in Heracles and paste it as the Sample ID in the Nanodrop program. (If available, a Cerner Accession Number or LabVantage ID could be used.)
9. Load 2 μL of the RNA sample and close the arm.
10. Click “RNA” on the sample type in the upper right-hard corner of the program.
11. Once the machine is finished reading the sample, read the A260/A280 ratio and the RNA concentration (ng/μL) measurements.
12. Clean the nucleic acid sample off the upper and lower pedestals with Kimwipe.
13. Save the nanoDrop report as a TSV file.
14. Close the program.
Note: *If sample fails to meet the minimum RNA purity or concentration, centrifuge the sample tube for 1 minute at maximum speed (>10,000×g), place the tube on ice and repeat the measurement process. If the sample continues to fail either the purity or concentration metric, the RNA sample is not suitable for analysis under the NanoString RNA STEP Assay protocol. Do not use RNA of insufficient quality or quantity in the assay.
   RNA Concentration and Integration with Heracles
1. Double click the Heracles desktop shortcut.
2. Under the “Testing” tab, click “RNA concentration”.
3. “RNA concentration readings” window pops up and click on the middle tab, “from TSV file”.
4. Press the light blue “1. Select TSV file” button to upload nanoDrop data in TSV file.
5. After pressing, “select TSV file, the “Lab_PMDL\nanoString_app” folder pops up. Next select the nanoDrop.tsv file you want to upload and press “Open”.
6. If there is an extra row like in the example below, click on the grey margin for the row to select the entire row. Then press “delete”. Duplicate rows must be deleted. You cannot have more than one record for each sample.
7. Press dark blue “3, transfer RNA concentration” button. Make sure that in the “Sample ID” box on the right, the correct form of sample ID is selected.
8. When the RNA concentration is successfully transferred, a small window pops up showing that the “RNA concentration was transferred.” Press “OK”.
   NanoString nCounter STEP Assay

The assay described below is for a 12-reaction run performed in two days.

### Reagents & Materials

| Item | Storage | | Expiration Date | |
|---|---|---|---|---|
| RNA samples: 37.5-75 ng/μL or 150-300 ng | −80° | C. | 10 | years |
| Universal RNA Control - Human Normal Tissue | −80° | C. | 2 | Years |
| Qiagen RNaseZap ™ RNase Decontamination Solution | 15-25° | C. | 6 | Months |

| | | | |
|---|---|---|---|
| TE Buffer, 1X Solution pH 8.0, Low EDTA | 2-8° | C. | Located on Container |

| Tween-20 | RT | None | |
|---|---|---|---|
| Nuclease-free water | RT | 1 | Year |

| | | | |
|---|---|---|---|
| Elements XT TagSet-192 (28 μL for 12 reactions) | −80° | C. | Located on Container |
| Elements XT TagSetEx24 (28 μL for 12 reactions) | −80° | C. | Located on Container |

nCounter Master Kit for 12 reactions which include:

| nCounter Prep Station Tips (1 each) | | | | |
|---|---|---|---|---|
| nCounter Cartridge Adhesive Cover (2 each) | | | | |
| nCounter Tip Sheaths (2 each) | | | | |
| nCounter Hybridization Buffer (1 × 580 μL) | 15-25° | C. | Located on Container | |
| 12-well Notched Strip Tubes (4 each) | | | | |
| 12-well Notched Strip Tube Lids (4 each) | | | | |
| nCounter Prep Plate (2 each) | 2-8° | C. | Located on Container | |
| nCounter Cartridge (1 each) | −80° | C. | Located on Container | |
| 96-well nanoString Plate Probe A1 Oligonucleotide | −80° | C. | 2 | Years |
| (T001-T096); (150 μL, 1 μM) | | | | |
| 96-well nanoString Plate Probe A2 Oligonucleotide | −80° | C. | 2 | Years |
| (T097-T192); (150 μL, 1 μM) | | | | |
| 96-well nanoString Plate Probe A3 Oligonucleotide | −80° | C. | 2 | Years |
| (T193-T216); (150 μL, 1 μM) | | | | |
| 96-well nanoString Plate Probe B1 Oligonucleotide | −80° | C. | 2 | Years |
| (T001-T096); (150 μL, 5 μM) | | | | |
| 96-well nanoString Plate Probe B2 Oligonucleotide | −80° | C. | 2 | Years |
| (T097-T192); (150 μL, 5 μM) | | | | |
| 96-well nanoString Plate Probe B3 Oligonucleotide | −80° | C. | 2 | Years |
| (T193-T216); (150 μL, 5 μM) | | | | |
| Master Probe A Stock (1 mL) | −80° | C. | 2 | Years |
| Master Probe B Stock (1 mL) | −80° | C. | 2 | Years |
| Master Probe Ext Stock A (1 mL) | −80° | C. | 2 | Years |
| Master Probe Ext Stock B (1 mL) | −80° | C. | 2 | Years |

### Equipment

Bio-Rad C1000 Touch Thermal Cycler
NanoString nCounter Flex Prep Station 5s
NanoString nCounter Flex Digital Analyzer 5s
Mini centrifuge
Benchmark Scientific StripSpin 12 microcentrifuge
Eppendorf Bench Top Centrifuge 5810
Micropipette and tips for 0.5-10 μL, 2-20 μL, and 20-200 μL
Nuclease-free 1.5 mL microcentrifuge tubes
PCR tube rack

### Day 1: Hybridization Step

### Oligonucleotide Probe Pooh Preparation

The oligonucleotide probes used with the nCounter Elements are formatted into 4 separate pools: (1) Probe A Master Stock (T001-T192); (2) Probe B Master Stock (T001-T192); (3) Probe Ext A Master Stock (T193-T216); and (4) Probe Ext B Master Stock (T193-T216). Pools are first created as Master Stocks and aliquot of each Master Stock is then diluted immediately before the hybridization reaction to create Working Pools. These Working Pools are subsequently added to the hybridization master mix. The concentration of the oligonucleotides in a Working Pool is different for each type of probe. Probes will be diluted further when added to the hybridization reaction. The procedure below shows the steps used to generate the Master Stocks and Working Pools for Probe A. Probe B, Probe Ext A and Probe Ext B.
Note: *The concentrations of each probe in the hybridization reaction are critical for maximizing the sensitivity of the reaction. Make sure to carefully follow appropriate pooling and dilution procedure to create accurate Working Pools.

### Master Stock Preparation

1. Remove the six 96-well Plates containing oligonucleotide Probes A1, Probes A2, Probes A3, Probes B1, Probes B2, and Probes B3 from the −80° C. freezer and thaw them on ice. Each oligonucleotide probe in each well is resuspended in 150 μL TE buffer (10 mM Tris pH 8, 1 mM EDTA). The concentration of each Probe A is 1 μM and 5 μM for Probe B.
2. Probe A Master Stock
   a. Pipet 5 μL of each Probe (1 μM) from each of the well of the 96-well plates labeled Probes A1 and Probes A2 into a 1.5 mL microcentrifuge tube. The total volume of probes added to the tube is 960 μL.
   b. Add 40 μL of TE buffer to a final volume of 1 mL. The final concentration of each Probe A in the Probe A Master Stock is 5 nM.
   c. Store in 4 μL aliquots at −80° C. freezer.
3. Probe B Master Stock
   a. Pipet 5 μL of each Probe (5 μM) from each of the well of the 96-well plates labeled Probes B1 and Probes B2 into a 1.5 mL microcentrifuge tube. The total volume of probes added to the tube is 960 μL.
   b. Add 40 μL of TE buffer to a final volume of 1 mL. The final concentration of each Probe B in the Probe B Master Stock is 25 nM.
   c. Store in 4 μL aliquots at −80° C. freezer.
4. Probe Extension A Master Stock
   a. Pipet 5 μL of each Probe (1 μM) from each well of the 96-well plate labeled Probe A3 into a 1.5 mL microcentrifuge tube. Only 24 wells in this plate are filled with oligonucleotides. The total volume of probes added to the tube is 120 μL.
   b. Add 880 μL of TE buffer to a final volume of 1 mL. The final concentration of each Probe Extension A Master Stock is 5 nM.
   c. Store in 4 μL aliquots at −80° C. freezer.
5. Probe Extension B Master Stock
   a. Pipet 5 μL of each Probe (5 μM) from each well of the 96-well plate labeled Probe B3 into a 1.5 mL microcentrifuge tube. Only 24 wells in this plate are filled with oligonucleotides. The total volume of probes added to the tube is 120 μL.
   b. Add 880 μL of TE buffer to a final volume of 1 mL. The final concentration of each Probe Extension B Master Stock is 25 nM.
   c. Store in 4 μL aliquots at −80° C. freezer.

Alternatively. Master Stocks in 1 mL volume can be ordered from IDT DNA. Make sure to specify a pool of Probe As at a final concentration of 5 nM per oligo, a separate pool of Probe Bs at final concentration of 25 nM per oligo, a separate pool of Probe Extension As at 5 nM per oligo and a separate pool of Probe Extension Bs at 25 nM per oligo.
Note: *Always create separate Master Stocks for Probe A, Probe B, Probe Ext A. and Probe Et B. Do not create combined Master Stock containing Probe A, Probe B. Probe Ext A. and Probe Ext B in the same tube; elevated background and lowered reaction sensitivity may result. *Never add a Master Stock directly to the hybridization Master Mix.*The probes in the Master Stocks must be appropriate for the targets being queried. If the reporter tags are reassigned to new targets, new Master Stocks containing the specific set of appropriate probes must be created. Do NOT add additional probes to existing Master Stocks. Always make separate Master Stock for the core TagSet and Extension Probes.*Minimize freeze-thaw cycles by preparing appropriate aliquots of the Master Stock and storing them in −80° C. freezer. Thaw each aliquot only once and then place in ice for use in creating Working Pools.

### Working Pool Preparation for 12 Reactions

1. Prepare TE-Tween buffer by adding 1 μL of Tween-20 to 1 mL of TE buffer (10 mM Tris pH 8, and 1 mM EDTA).
2. Probe A Working Pool
   a. Take out Probe A Master Mix aliquot from the −80° C. freezer and thaw on ice.
   b. Prepare an 8.3-fold dilution of Probe A Master Stock to generate Working Pool Probe A. Add 29 μL TE-Tween (10 mM Tris pH 8, 1 mM EDTA, 0.1% Tween-20) buffer to the 4 μL aliquot of Probe A Master Stock to a final volume of 33 μL. The concentration of each Probe A in the Working Pool A is 0.6 nM.
   c. Mix well and spin down contents to the bottom of the tube.
   d. Keep the tube on ice if not used immediately for the next step.
3. Probe B Working Pool
   a. Take out Probe B Master Mix aliquot from the −80° C. freezer and thaw on ice.
   b. Prepare an 8.3-fold dilution of Probe B Master Stock to generate a Working Pool Probe B. Add 29 μL TE-Tween (10 mM Tris pH 8, 1 mM EDTA, 0.1% Tween-20) buffer to the 4 μL aliquot of Probe B Master Stock to a final volume of 33 μL. The concentration of each Probe B in the Working Pool B is 3 nM.
   c. Mix well and spin down contents to the bottom of the tube.
   d. Keep the tube on ice if not used immediately for the next step.
4. Probe Extension A Working Pool
   a. Take out Probe Extension A Master Mix aliquot from the −80° C. freezer and thaw on ice.
   b. Prepare an 8.3-fold dilution of Probe Extension A Master Stock to generate a Working Pool Probe Extension A. Add 29 μL TE-Tween (10 mM Tris pH 8, 1 mM EDTA, 0.1% Tween-20) buffer to the 4 μL aliquot of Probe Extension A Master Stock to a final volume of 33 μL. The concentration of each Probe Extension A in the Working Pool Extension A is 0.6 nM.
   c. Mix well and spin down contents to the bottom of the tube.
   d. Keep the tube on ice if not used immediately for the next step.
5. Probe Extension B Working Pool
   a. Take out Probe Extension B Master Mix aliquot from the −80° C. freezer and thaw on ice.
   b. Prepare an 8.3-fold dilution of Probe Extension B Master Stock to generate a Working Pool Probe Extension B. Add 29 μL TE-Tween (10 mM Tris pH 8, 1 mM EDTA, 0.1% Tween-20) to the 4 μL aliquot of Probe Extension B Master Stock to a final volume of 33 μL. The concentration of each Probe B in the Working Pool Extension B is 3 nM.
   c. Mix well and spin down contents to the bottom of the tube.
   d. Keep the tube on ice if not used immediately for the next step.
Note: *Always create separate Probe A, Probe B. Probe Ext A and Probe Ext B Working Probe Pools. Do NOT create a combined Working Probe Pool containing Probes A, B, Ext A, and Ext B in the same tube; elevated background and lowered sensitivity may result. *Long-term storage and reuse of Working Pools are not recommended. Discard Working Pool after use. Fresh dilution of each Master Stock should be made for subsequent hybridization.

### RNA Hybridization Reactions

RNA samples are used as input in the nCounter hybridization reaction containing the NanoString TagSet (Barcoded Reporter and Capture Probes). The overnight hybridization reactions enable specific hybridization of reporter and capture probes to their target-specific oligonucleotide and probes (A. B. Extension A and Extension B).

### Important Probe Handling Instructions:

Do not vortex or pipette vigorously to mix
Mixing should only be done by flicking or inverting the tubes
Do not spin down tubes faster than 1,000 rpm for more than 30 seconds if using a microcentrifuge
Do not “pulse” to spin because it will cause the centrifuge to go to maximum speed and may spin the probes out of solution
Check the reagent labels before you begin to ensure the correct reagents are being utilized

### Hybridization Set Up

1. Preheat thermal cycler to 67° C. with a heated lid at 72° C. to use 15 μL volume.
Note: *NanoString recommends a thermal cycler with a programmable heated lid for this protocol. Models without programmable lids may reach a high temperature that can cause tubes to melt or deform during overnight hybridization. If non-programmable thermal cycler is used, make sure that the heated lid does not exceed 110° C.
2. Remove XT TagSet-192, XT TagSet Ex24, aliquots of Master Stocks Probe A, Probe B. Probe Ext A and Probes Ext B, Universal RNA control and RNA samples from the −80° C. freezer and thaw on ice. Invert or flick the tubes several times to mix well and briefly spin down.
3. Create Working Pool of all the Master Stocks by adding 29 μL of TE-Tween (10 mM Tris pH 8, 1 mM EDTA, 0.1% Tween-20) buffer directly to each tube. Detailed procedure for this step is outline in the previous section under “Working Pool Preparation for 12 Reactions”.
Note: *Diluted probe pools should not be stored for long term use.
4. This hybridization procedure is for 12 reactions but a Master Mix enough for 14 reactions is prepared to allow 2 reactions worth of dead volume. Master Mix is prepared to minimize pipetting and ensure uniformity in each tube.

### Hybridization Master Mix Preparation:

a. To the tube containing 28 μL XT TagSet 192, add the following:
70 μL NanoString hybridization buffer
28 μL XT TagSet Ex24
7 μL Probe A Working Pool
   7 μL Probe Ext A Working Pool
b. Flick or invert the tube repeatedly to mix then briefly spin down the Master Mix.
c. To the Master Mix above, add the following:
7 μL Probe B
7 μL Probe Ext B
d. Flick or invert the tube repeatedly to mix then briefly spin down the Master Mix. The total volume in the hybridization Master Mix is 154 μL.
5. Label the NanoString 12-well Notched Strip.
6. Prepare hybridization reactions as follows:
a. Add 11 μL of the hybridization Master Mix to each well of the strip tube. Use fresh tip each time for pipetting into each well to accurately measure the correct volume.
b. Add 4 μL of RNA sample to each tube containing the master mix. If RNA sample volume is less than 4 μL, add RNase-free water to each tube to bring volume of each reaction to 15 μL. Total volume in each tube is 15 μL.
Note: *NanoString recommends between 150-300 ng of RNA if derived from FFPE.
c. Cap the 12-well notched strip tightly with the lid and mix them by inverting the tubes several times and flicking to ensure complete mixing.
d. Spin briefly in the StripSpin 12 microcentrifuge.
7. Immediately place the tubes in a preheated 67° C. thermal cycler.
8. Incubate hybridization reactions at 67° C. with heated lid at 72° C. for 24 hours. To minimize the potential for evaporation, thermal cycler is set at 5° C. above the heat block temperature. Maximum hybridization time should not exceed 30 hours.
9. Ramp down reaction to 4° C. when complete and process the following day. Do not leave reactions at 4° C. for more than 24 hours or increased background may result.
Note: *Selecting a fixed hybridization time followed by a ramp down to 4° C. ensures equivalent hybridization time for all assays being compared in the same series of experiments. In this protocol, hybridization time is fixed at 24 hours. Counts continue to accumulate with time at 67° C., with total counts typically increasing 5% per hour between 16 and 24 hours. Although a 16-hour incubation is adequate for most purposes, a longer incubation increases sensitivity by increasing counts without significantly increasing background.
10. Once the hybridization reactions have been removed from the thermal cycler, proceed immediately to the nCounter Prep Station protocol described in the following step.

### Day 2: Purification Step in the Ncounter Flex Prep Station

The Prep Station is a multichannel pipetting robot that processes samples to prepare them for data collection on the nCounter Flex Digital Analyzer. The instrument performs liquid transfers, magnetic bead separations, and immobilization of molecular labels on the sample cartridge surface. All consumable components and reagents required for sample processing on the Prep Station are provided in the nCounter Master Kit and must be loaded onto the Prep Station deck prior to use. No reagent preparation or dilutions are required. The Prep Station can process up to 12 lanes per run in approximately 3 hours.

### Operating the Prep Station

Prior to Initiating a Run.

Prior to starting a new run, ensure that the waste containers have been emptied. Empty waste containers are required for every run.
1. Remove the combined waste receptacle by lifting it straight up and out of the Prep Station.
2. Remove the liquid waste container from the combined receptacle by using the latch on the front and dispose of the liquid appropriately.
3. Verify that the plastic rack holding the used piercers, tip sheaths, prep plates, and strip tubes from the previous run have all been removed from the deck.
Note: *If waste containers are not emptied, tips could come into contact with waste liquids and contaminate samples, or excess tips could pile up and cause a system malfunction. *Used plastic ware, such as reagents, cartridges, and pipet tips, must be collected and disposed of properly in accordance with local safety regulations and laboratory procedures.*Do not dispose of biohazard samples in the sink or drain. Dispose of all samples in accordance with local safety regulations and laboratory procedures.

### Initiating a Run.

This workflow begins after the samples have been hybridized overnight (see Hybridization Set Up procedure above). On the Prep Station deck, hybridized samples are purified and immobilized in a Sample Cartridge.
1. After turning on the NanoString nCounter Flex Prep Station, the “Select Instrument Mode” screen appears. This screen asks the user to select either Diagnostics mode (blue, on the left) or Life Sciences mode (green, on the right). Press the green icon labeled NanoString Life Sciences to enter Life Sciences mode. The system will load the application and present the Main Menu.
2. To set up a new run, touch “start processing” from the main menu.
3. The “Select Protocol” screen appears. Select “High Sensitivity” protocol. The high sensitivity protocol increases the binding of all molecules to the cartridge by about 2-fold versus prior protocols and adds an extra 30 minutes to the processing time. Press “next”.
4. The “Sample Selection” screen appears. Select the sample positions that will be processed. Blue tubes will be processed, and grey tubes will not be processed. If processing 12 samples, press “select all”. Then press “next”.
5. The “Warm Reagents & Cartridge” screen appears. nCounter Cartridges and Prep Plates must be at room temperature prior to processing.
   a. Remove one nCounter blank sample cartridge from the −80° C. freezer. Allow it to warm to room temperature for 20 minutes before removing from the foil package. To prevent condensation on the cartridge, do not remove the cartridge pouch until it has reached room temperature.
   b. Remove two nCounter Prep Plates from the 4° C. fridge. Centrifuge at 2000 g for 2 minutes to collect all liquids in the bottom of the wells. After centrifugation, visually inspect plates to ensure that reagents have collected at the bottom of each well. Confirm for the presence of magnetic beads in the last row of the plate (row H). Allow the plates to warm to room temperature for 20 minutes.
Note: *If the cartridge and Prep Plates are not at room temperature prior to use, assay variability may increase.
6. After 20 minutes when the cartridge and Prep Plates warm up to room temperature, press “next” on the screen.
7. The “Waste Receptacles” screen appears. See previous section on “Prior to Initiating a Run” for the waste removal procedure. Press “next”.
8. The “Reagent Plate” screen appears. Remove the clear plastic lids and place the Prep Plates on the deck. Make sure plates are oriented with the label facing the operator and that they are flush with the deck surface by aligning the plates on the position pin and pressing down firmly near the plate catch. Do not remove the foil or pierce the wells on the reagent plate. The Prep Station will pierce the wells during processing. Press “next”.
Note: *If the plate is placed incorrectly, the Prep Station will pause the protocol until the user intervenes.
9. The “Tips & Foil Piercers” screen appears. Remove the metal tip carrier from the Prep Station deck by lifting straight up. Place the tips and the foil piercers into the carrier. It is helpful to place the carrier at eye level to align the plastic tips in the carrier. The shorter, dark grey foil piercers should be in the front. Place the loaded tip carrier back into the Prep Station deck with the short dark grey foil piercers facing front. Press “next”.
10. The “Tip Sheaths” screen appears. Tip sheaths are used to reduce the amount of consumable waste. They allow the system to dedicate tips to a set of 6 samples and store them while processing the other 6 samples. Place the tip sheaths on the deck and press firmly into place. Press “next”.
11. The “Sample Cartridge” screen appears. Carefully place the nCounter cartridge under the electrode fixture. Make sure that it is seated completely in the machine depression. If not seated properly, the electrodes may become bent. Press “next”.
12. The “Electrode Fixture” screen appears. Carefully lower the electrode fixture in place over the cartridge. The 24 electrodes should insert into the 24 wells. Press “next”. Do not use the release handle while lowering the fixture. Doing so will prevent the fixture from locking. Press on the body of the fixture away from the release handle. Press “next”.
Note: * If any resistance is felt while lowering the fixture, stop and adjust the position of the cartridge slightly. Make sure the electrodes are correctly aligned. The Prep Station will not be able to process any of the samples if there are bent electrodes.
13. The “Empty Strip Tubes” screen appears. Place the two 12-well empty strip tubes without the lids on the deck. Press “next”. Only use strip tubes provided by NanoSuting. Other tubes have different dimensions and will cause system failure.
14. The “Hybridized Samples” screen appears. Lift the lid and place the hybridized sample strip tube from the thermal cycler on the deck of the Prep Station, ensuring the tube 1 aligns with position 1. Ensure that the tube lids are removed from the hybridized samples prior to placing tubes on the deck. Leaving the caps on will result in a pause in the protocol that requires user intervention. Note that the strip tube is asymmetrically keyed, and if the strip tube is placed incorrectly, the lid won't close properly, and the Prep Station will not be able to start processing. Hybridized strip tubes have 2 notches to ensure proper orientation. Press “next”.
15. The “Notification Options” screen appears. Select the e-mail addresses where error and completion confirmation emails should be sent. To add a new address, press “add email”, type in the address and press “enter”. Finally select whether the Prep Station should make an audible alarm when processing is finished. When all alerts have been set, press “next”.
16. The “Start Processing” screen appears. Press “start” when ready to begin processing.
17. The “Validating deck layout” screen appears. The nCounter Prep Station will first check that all consumables and reagents have been placed properly on the deck. To do this, the Prep Station confirms that the sensors for the sample cartridge, electrode fixture, and heated lid are all in the correct state. The pipette head then checks that tips, tip sheaths, strip tubes, and Prep Plates are all in place by touching them with a set of validation tips. Do not be alarmed that the Prep Station is touching the consumables. This is part of normal operation. If the Prep Station determines that a consumable is misplaced, it will instruct the user to adjust the configuration.

The “Validation deck layout” screen will eventually update to the “System Processing” screen. Both screens display the current time of day and the estimated time of day that the run will complete. They also provide the option to pause the run.
Note: *It is advised to not abort and re-start the run when there is a deck layout failure. Depending on the progress of the validation process, the Prep Plates may have been pierced and liquid handling may have begun. Reagents may need to be replaced before re-starting the run. *Stay with the Prep Station while the deck layout is being validated. Any problems that may be encountered during this step will require user intervention to proceed.
18. When the run is complete, the blue “System Processing Complete” screen appears, and the time will count up. The Prep Station purification process takes about 3 hours to finish. Press “next”.
19. The “Run Successfully Complete” screen appears. It lists the steps to follow once the run is complete, including:
   a. Remove and discard empty reagent plates.
   b. Remove and discard the empty tip racks and foil piercers.
   c. Remove and discard the sample strips.
   d. Remove the sample cartridge and seal the wells.
20. To release the fixture after the run is complete, press the lever in the center top of the device towards the front with a finger.
21. After processing is complete, it is important to do the following:
   a. Seal the wells immediately with the adhesive film included in the nCounter Master kit to prevent evaporation. Do not leave processed cartridge uncovered overnight, as evaporation of liquid in the wells results in data loss. Proceed to the Digital Analyzer Procedure.
   b. If sample is not used immediately in the next step. Samples should be protected from as much light as possible. Store them in dark to prevent photobleaching.
   c. Store samples in the refrigerator at 4° C. Once sealed, samples can be stored at 4° C. for up to a week with minimal degradation.
   d. Empty the waste receptacle and discard the consumables appropriately.
22. Press “finish” to return to the Main Menu.
   Digital Counting in the nCounter Flex Digital Analyzer

There are 3 types of files used by the nCounter Analysis System: Reporter Library File (RLF). Cartridge Definition File (CDF), and Reporter Code Count (RCC) File.
The Reporter Library File is generated by NanoString and is unique to each custom CodeSet. It contains the information used during image processing to assign target identities to the barcodes.
The Cartridge Definition File is created by the user. It defines assay-specific data to associate with the data output and the parameters used by the Digital Analyzer during image collection and processing. Data contained in the CDF include the following:
   Lane ID: The lane ID column defines which flow cells in the cartridge will be scanned. If all 12 lanes will be scanned, this should not be changed. If only a subset of lanes will be scanned, the information for the empty lanes can be deleted.
   Sample ID: This column is where the user may name individual samples. In this assay, for the Sample ID we use MRN-FK_Sample.
   Owner ID: This is an optional field that can be named; information is output with the data.
   Comments: Enter additional sample or experimental details in the Comments field; information is output with the data.
   Date: The date information is optional. The date of a scan is automatically added to the beginning of the RCC file name, so it is not required to be included here.
   FOVCount: This field specifies the number of images (field of view) to analyze per assay, which corresponds to the amount of data to collect, 280 FOV (high resolution) is used in the STEP assay.
   GeneRLF: This field defines the reporter library file to associate with the data. It is extremely important that this filename be correct or data will be misinterpreted. The “.rlf” file type extension should not be used here.
   A sample of the CDF is shown in FIG. 2.

FIG. 2. A sample of CDF viewed on notepad.
The Reporter Code Count file is generated by the Digital Analyzer. Each one contains the data for one of the twelve lanes (assays) in a cartridge, detailing the number of counts for each target in an assay.

### Starting a Run

1. Go back to the Heracles main menu: “Testing”/“Start Run”. Start Run window will appear.
2. Press gray “see pending runs” button.
3. After pressing, “see pending runs”, any pending runs will show up as shown below. The run number is under the column PK and the custom panel number is under the “ReagentLot” column.

Select the pending run you want to check. It will be highlighted in blue. Check the “see all fields” box if necessary. Press the purple “run samples” button to see the samples in the run.
4. After pressing, “run samples” button, the data associated with that particular run will be shown in a table. The RNA concentration and purity uploaded earlier are transferred to the table as shown below.
5. Press the yellow “start run” button.

When the “start run” is successful, a cdf file is created for that run. A small window pops up showing where the cdf file is saved. (This is the location for the FTP transfer.) A new folder with the associated run number is created in the main laboratory PMDL directory: M:\lab\Lab_PMDL\Validation_RNA expression panel\Custom_Panel\NGS_Amp\.
6. Press “OK”.
7. Go to the network directory and locate the new folder created (i.e., 1025). Open the newly created folder. The folder name is the run number.
8. Make sure that the cdf file is saved in the folder associated with that particular run before transferring the cdf file to the nanoString Digital Imager.

### Uploading Files to the Digital Analyzer

1. Transfer of the cdf file from the network to the nanoString Digital Analyzer is done thru the FTP software, WinSCP. On the desktop screen, double click the “WinSCP” shortcut icon.
2. After opening the WinSCP icon, the Login window pops up. On the left side of the Login window, click on “nanoString” and press “Login”.

After logging in, WinSCP main menu pops up. The window is divided into two: on the left side is the computer network directory and on the right side is the nanoString Digital Analyzer directory. The Digital Analyzer has 3 main folders: CDFData, RCCData, and RLF. Go to the CDFData folder and drag the cdf file from the left side to the CDFData folder.

### Initiating a Run in the Digital Analyzer

1. Turn on the nCounter Flex Digital Analyzer and the “Select Instrument Mode” appears. This screen asks the user to select either Diagnostic mode (blue, on left) or Life Sciences mode (green, on the [symbol]
2. Press the green icon labeled “NanoString Life Sciences” to enter Life Sciences mode.
3. Insert USB into the port off the right side of the Digital Analyzer touch screen. The Upload Files screen appears. Press “upload RLF” and “Select RLF Source” screen appears. RLF files already in the system as well as the RLF files found on the USB flash drive that have not already been uploaded will be displayed. Select the RLF file provided by the NanoString Bioinformatics group for the STEP assay, “MCC_Boyle_3_C3603+A3604.RLF” to upload the file. The RLF will be saved on the Digital Analyzer. Press “next”.
4. From the Main Menu, select “start counting”.
5. The “Select Stage Position” screen appears. Select a stage position on the touch screen. The stage position is where you are going to insert the cartridge in the digital analyzer and where you will also enter the information. The selected cartridge will appear in green. If the wrong cartridge is selected, touch the correct position and the active cartridge will display in the new position. Insert the cartridge in the selected stage position while holding it lengthwise. Make sure the cartridge is seated flat in the slot. Close the magnetic clip gently and push down to ensure that the cartridge is flat. Press “next”.
6. The “Select Cartridge Definition Mode” screen will appear. Since the CDF was already uploaded to the Digital Analyzer using FTP, press “load existing” on the screen.
7. The “select CDF” screen appears. Select the CDF to be used in this assay and press “next”.
8. The “Cartridge Information” screen appears. The information from the CDF will be populated in this screen. To change any of the information specified for the cartridge, press the appropriate fields on the screen. Press “done”.
9. The “Select Stage Position” screen will appear again.
10. For additional cartridges, press “next” to repeat the process until all cartridges for the runs are defined. If there is only one run, press “done”.
11. The “Initiating Imaging” screen appears. Press “start” to start the scan.
12. Once the imaging has begun, the door will remain locked until the system is paused or the run is complete.
13. The “Counting Cartridge ID” screen will display the following information:
   The cartridge ID for the active cartridge (the cartridge currently being scanned)
   Real time Cartridge scan status/progress:
   Blue—cartridge scan completed and/or in progress
   Green—cartridge yet to be scanned during the run
   Clear (white)—cartridge position for which no data has been defined
   Current time—the current time of day as defined in the system setup utility
   Time left (#)—the approximate amount of time to complete the active cartridge
   Time left (all)—the amount of time to complete all cartridges
   Finish time—the time of day the run will be finished (current time+total run time)
14. Once the scan is completed, the .RCC output tiles can be transferred from the Digital Analyzer to the computer via FTP.
15. Press the “WinSCP” icon on the computer desktop. The login window appears. Press “NanoString” located on the lower left of the login window. Then press “Login”.
16. After the run, each sample in the assay will have a RCC file. The RCC files are automatically saved on the RCCData directory. The output or RCC files for each run is saved in a compressed zip folder. To transfer the RCC folder, drag the RCC folder on the right side to the computer directory on the left side.
   Data Analysis in nSolver 4.0
1. Open the nSolver Analysis Software.
2. Click yellow “Import RCC files” button.
3. Import RCC files window pop up. Click “browse” to select the RCC directory and click “open”. Press “next”.
4. After the RCC files been uploaded, highlight all files that need to be QC'ed. Then click “QC” icon.
5. Run QC window pops up. Check the following: Execute System QC on files, Imaging QC (75), Binding Density QC (0.1-2.25), Positive Control Linearity QC (0.95), Execute QC on mRNA data, Positive Control Limit of Detection QC (Flag lanes when 5 tM positive control is less than or equal to: 2). Press “Run QC” to generate raw and normalized files. Check for any QC flags in these files. Consult lab manager with any concerns.
6. On the main menu, go to the “Experiments” menu and scroll down to “New Experiment”.
7. “Experiment Design Wizard” pops up. This wizard guides you through the steps necessary to define your experiment, normalize your data, and create fold change estimates. Fill in the required fields and press “next”.
8. “Add samples/lanes” pops up. On the left hand side, on “select analyte type” section, scroll down to select “mRNA”. Click “MCC_Boyle_3_C3603+A3604” to select this RLF. After the RLF is selected, all samples that were ran using this RLF will show on the right side.
9. Select the files needed for the analysis by clicking on the file name cells or alternatively, use “shift+down arrow” to highlight files needed for analysis. Then click on the “Keep Selected” icon. Only the selected files will appear on the screen. Press “next”.
10. “Add Sample Annotation” window pops up.
11. Click “Add annotation” icon.
12. New annotation column appears. Type “UNK” for test samples and “Ctrl” for control sample in the annotation column. Press “Next”.
13. “Select Background Subtraction OR Background thresholding Parameters” windows pops up and then click “Next”.
14. “Normalization Parameters” window pops up.
   a. On the left hand side, check the “1. Positive Control Normalization” box and select all Positive controls. Select “geometric mean” to compute for normalization factor. Flag lanes if normalization factor is outside of the 0.3-3 range.
   b. On the right hand side, check the “2. CodeSet Content (Reference or Housekeeping) Normalization” box. Select “standard”. Under Normalization Codes, discard housekeeping genes with Avg Counts lower than 100 by clicking the left arrow to transfer it to the CodeSet content genes. Housekeeping genes that also have extremely high or low % CV are discarded and transferred to the Codeset content section. Select “geometric mean” to compute for normalization factor. Flag lanes if normalization is outside of the 0.1-10 range. Press “Next”.
15. Fold Change Estimation window pops up. Click on “Build Ratios” box and select “Using user selected reference samples. On the left hand side under All Samples section, highlight the “CTRL” and transfer to the “Base Samples” section by clicking the right arrow. Click “Next”.
16. “Ratio Data Names” window shows up. Highlight “UNK vs Reference”. Click “Finish”.
17. Go back to the nSolver main menu and on the left hand side, click on the “Experiments” tab. Click on the “Raw Data” directory and all raw data will be displayed on the right hand side. Flags will appear under the QC metrics that did not meet the QC standards.
18. Click the 12 box next to the File Name to highlight all the samples. Then click “Advanced Analysis”.
19. “Select Advanced Analysis” window pops up. Enter the Name of the analysis. Click “browse” to specify the path where you want the analysis data to be saved. Press “Next”.
20. “View and select sample annotation information to be used in covariates in analysis” window pops up. Select “Description” under “Identitiers” and select “New Annotation” under “Use in Analysis”. Press “Next”.
21. “nCounter Advanced Analysis—Options” windows appears and under “Analysis Type”, click “Custom Analysis”. It will turn green. Upload the probe annotation file for this particular panel by clicking on “here” located at the lower right hand corner of the screen. The annotation file is from the NanoString bioinformatics group. Each panel has its own probe annotation file.
22. Under General Options: Check or select (a) Experiment Type: Standard, (b) Choose modules to run: Overview, Normalization, and Pathway Scoring, (c) Choose an annotation for defining probe sets: Probe.Annotation, (d) Choose additional image types to create: None and (e) check box for Omit Low count data.
23. Under Normalized Data, Select Normalize mRNA and automatically find good normalization probes.
24. Under Pathway Scoring, Select “New Annotation” under “Available Annotations” and transfer to “Plot Pathway Scores Vs”. Press “Finish”.
25. Screen goes back to the experiment menu/Analysis Data. Highlight the analysis name and click “Analysis Data”.
26. A new html page shows up that has all the analysis results: Heatmaps. PCA, Study Design. and Other QC. The advanced analysis data is also saved in the laboratory PMDL drive: M:\lab\Lab_PMDL\Validation_RNA expression panel\Custom_Panel\NGS_Amp.

### Transfer of Nsolver Files to Heracles

1. HERACLES Menu: Testing/Read nSolver files.
2. Click green “select run directory” button.
3. Browse in the directory tree to find corresponding folder for the run. For example: M:\lab\Lab_PMDL\Validation_RNA expression panel\Custom_Panel\NGS_Amp\025\Run025 2021-11-17 11-32 Do not select the ‘ . . . RCC’ folder, used for the nSolver analysis.
4. After selecting the correct folder click “OK”. Double check that the correct folder has been selected.
5. Click yellow “read files for run” button. Several files will be read and listed in the text box beneath. This process takes many seconds, but less than 1 min. Click ‘OK’ in the message window when done.

### Review Results

1. HERACLES Menu: See data/Run data
2. In Run Data window, in the ‘run samples’ tab: click gray “see runs” button.
3. Select run and click purple “run samples” button.
4. Select case and click yellow “data by MRN” button.
5. In the Data by MRN window, select ‘nanoString-STEP’ tab: select sample in top left grid and click light yellow “see results” button.
6. Results will appear in the other grids in the window with all results in the right grid, upregulated genes in the bottom left grid, and downregulated genes in bottom center grid.

### Assign Cases to Pathologist to Create/Sign-Out/Communicate Reports

1. Technologist notifies pathologist by e-mail that run is complete and results are ready for sign-out.
2. Pathologist opens Heracles and repeats review of results steps above.
3. In the ‘Data by MRN’ window, pathologist clicks yellow “create report” button to sign out case.
4. HTML report is automatically generated in M:\lab\Lab_PMDL\Validation_RNA expression\Custom_Panel\NGS_Amp\REPORTS.
5. To generate a pdf report, click gray “create pdf report” button. The file also is saved automatically in the above PMDL folder.
6. Pathologist checks report and communicates with ordering provider.

### 6. Example 5

Detection of Gene Expression with a Custom-Designed RAN Salah Targeted Expression Panel (STEP) Using the Nanostring Platform

This Report describes the experiments that were carried out in laboratory to validate the RNA STEP (Salah Targeted Expression Panel), a gene expression-based assay that interrogates normalized expression of 204 genes in clinical samples. RNA STEP uses the NanoString nCounter instrument which is an amplification-free, multiplexed, automated RNA profiling platform that is optimized for use with mRNA extracted from formalin fixed paraffin embedded (FFPE) samples. The Nanostring platform is particularly known for its ability to produce robust and accurate results with small amounts of degraded mRNA and high flexibility regarding gene design (1-5). The assay is based on counting the number of mRNA molecules with specific probes designed for each gene. Changes in gene expression in tumor samples relative to a pooled RNA control sample from healthy adults (BioChain Institute. Inc) are detected, including detection of MET exon 14 skipping.

The 204 gene design of RNA STEP was based on feedback from Moffitt clinicians about which genes are most needed for clinical trial screening (Table D). Moffitt Cancer Center has multiple and changing clinical trials for patients with cancer, many with biomarker-based inclusion and exclusion criteria. The results of RNA STEP testing provide complementary information to NGS testing using remnant mRNA from NGS testing.

This ability of the assay to use remnant RNA from other clinical testing helps preserve clinical tissue for potential future needs. With the ability to add gene expression information to NGS results about tumors, oncologists and patients will be better informed to make decisions about which clinical trials in the plethora of trial available at Moffitt to consider for enrollment. This validation of the RNA STEP assay is intended for it to be integrated into the test menu of molecular assays offered by the CLIA Advanced Diagnostics Molecular Laboratory. The RNA STEP assay validation was guided by the joint consensus recommendations for validation of next generation sequencing (NGS) assays by the Association for Molecular Pathology and College of American Pathologists (6-9) because the complexity with the high number of markets analyzed is comparable. We also referred to CAP All Common and Molecular Pathology checklist items for validation requirements.

The present validation established the Assay performance characteristics as described in the sections that follow:
1. Analytical validation
   1.1. Sensitivity (LOD, Limit of Detection)
   1.2. Precision
      1.2.1. Operator-to-Operator variability
      1.2.2. Day-to-day variability
      1.2.3. Instrument-to instrument variability
      1.2.4. Lot-to-Lot variability
   1.3 Specificity (Interfering substances)
2. Diagnostic validation (concordance study)
   2.1. Accuracy, Positive Predictive Values, Negative Predictive Values
   2.2. Reference range
   2.3. Reportable range

To validate RNA STEP, 102 independent clinical remnant mRNA samples were tested that were originally extracted from FFPE clinical tissue for Moffitt STAR NGS testing (Illumina TruSight Tumor 170 platform, CLIA lab developed procedure). The STAR NGS and RNA STEP assays detect changes in 89 overlapping genes, including MET exon 14 skipping. Of the 59 genes with DNA-based amplification detection by STAR NGS, 38 genes are mutually covered by the nanoString assay. STAR NGS mRNA clinical samples are extracted from a variety of FFPE sample type, including resected specimens, core biopsies, and pleural fluid and fine needle aspiration (FNA) cell blocks. A subset of 19 of these 102 clinical samples had paired results from STAR NGS and a different 760 gene commercial nanoString panel (TS360). A pooled RNA control sample derived from tissue from 10 healthy human adults (BioChain Institute, Inc. catalog number: R4234565, Lot C5 1095 was included in each run tot comparison and/or a non-tumor lung sample. Lung cancer (N=25), ovarian cancer (N=2) and normal spleen (N=1) cell lines were also tested with mRNA derived from cell pellets, 25 mRNA samples derived from frozen tissue with panted RNAseq result, and 28 high grade serous ovarian cancer samples derived front flash frozen rumor.

RNA was extracted front FFPE tissue specimens according to the Qiagen DNA and RNA AllPrep protocol using the automated Qiacube instrument for clinical tissue samples. Subset of samples were extracted with an RNA only extraction protocol, the RNeaay Plus Minikit (Qiagen), to confirm that RNA extracted with different RNA extraction protocols can be used The STAR NGS assay requires FFPE tissue with at least 10% tumor cellularity, a minimum of 50 tumor cells per H&E slide evaluation, and tissue from 5-10 unstained slides with 7 μm thickness. The guidelines for tissue adequacy for RNA STEP will mirror these STAR NGS adequacy guideline since remnant RNA from STAR NGS will be the main source material for RNA STEP clinical testing.

The run data was processed with nSolver 4.0 advanced analysis software and transferred to a custom internal SQL-hued database, Heracles. The log 2 ratio for each gene was calculated by comparison of the normalized log 2 test sample relative to the normalized log 2 count of a pooled normal control. The data was filtered and reported as a list of upregulated and downregulated genes with log 2 ratios greater or equal than 2 and less than or equal to −2, respectively. The lists of upregulated and downregulated genes are followed by a list of all genes in the panel with their log 2 ratio. This report will be reviewed and signed out by Moffitt pathologists.

To investigate the analytical sensitivity (limit of detection) of the RNA STEP assay, one cell line and two clinical RNA specimens representing MDM2, CDK4, and KRAS gene upregulation and MET exon 14 skipping were diluted with water to different concentrations (30 ng, 200 ng, 100 ng, 50 ng, 25 ng, 10 ng). One diluted sample had MET exon 14 skipping und low tumor cellularity (20%). The lower limit of detection (LLOD) was determined based on the sample passing quality control metrics during processing, and the ability to detect increased gene expression concordant with gene amplification or MET exon 14 skipping using the normalized log 2 ratio (log 2 ratio≥2 for increased expression) relative to the pooled RNA control sample. A log 2 ratio of 2 is equivalent to gene expression in the clinical sample with a 4 fold increase compared to the same gene in the control sample.

Results (log 2 ratio per gene) from diluted samples were analyzed for concordance with undiluted samples. All results tot the two clinical samples with 80% and 210% tumor cellularity diluted down to 10 ng were concordantly positive with the undiluted samples tor MET exon 14 skipping, MDM2, and CDK4, and KRAS, except 2 results or KRAS at 200 ng and 100 ng had a log 2 ratio at 1.9, just below the positive cut-off of 2 (Table 3, A and B). The log 2 ratio for KRAS with 300 ng input for 3 tests on different runs ranged from 2.0 to 2.6 (average 2.4) Person correlation was performed to compare the results, log 2 ratios of the 204 genes, from the 2 diluted samples. For the sample with 80% tumor cellularity, the correlation was >0.9 even down to 10 ng (Table 4A). For the sample with 20% tumor cellularity, the correlation (r) decreased to 0.45 with input of 10 ng, respectively (Table 43).

Additionally, 8 clinical samples tested with gene amplification and inherently low tumor cellularity (10-30%) and/or extracted RNA concentration (<150 ng, range 13.2 ng to 124 ng) to confirm that gene upregulation may be detected accurately in clinical samples with inherently lower cellularity or RNA amount (Table (5A). With low tumor cellularity (N=8) and/or low RNA amount (N=4 with RNA<150 ng) in these samples, gene upregulation was detected in 8 of the 13 amplified genes (PPA=61.5%, log 2 ratio cut-off≥2), lower than the PPA for clinical samples with <30% tumor cellularity (PPA=70.4% (76/108). With a lower cut-off of log 2 ratio ≥1, equivalent to 2 fold increased expression relative to the control sample, the PPA was higher at 76.9% (10 of 13). In the sample with lowest input of 13.2 ng of mRNA, NGS identified amplification in 2 genes. RNA STEP was concordantly positive for one gene (CDK4) and discordantly negative the other (EGFR). Three clinical samples with low tumor cellularity and MET exon 14 skipping by NGS were analyzed (Table 5B). All 3 samples were concordantly positive for MET exon 14 skipping with RNA STEP with log 2 ratios well above the positive cut-off of ≥2.

Given the results of the LLOD dilation experiments and additional experiments with clinical samples with inherently low tumor cellularity of RNA amount, the lower limit of detection was placed with inputs of 10 ng and 10% tumor cellularity. Due to the lower PPA with lower tumor cellularity and/or RNA input amount (62.4%), a bolded qualifying statement was added to clinical reports from testing of samples with ≥30% tumor cellularity or ≥150 ng input amounts to explain the higher chance for false negative results.

The precision of the RNA step assay was determined by multiple repeats of testing of samples within runs and between runs with comparison of results from different operators (technologists), days, instruments (PCR and nanoString), and reagent lot numbers.

Data between replicates for all comparisons were analyzed with Pearson correlation. The results from all comparisons demonstrated excellent correlation (r>0.95 for all comparisons). The RNA STEP assay demonstrated returns similar results regardless of variations in testing conditions. Analysis of operator-to-operator variability was performed by two technologists, who tested the same 11 diagnostic samples and 1 control in independent runs. Minimal operator-to-operator variability was detected with excellent correlation between gene log 2 ratio results for all duplicate samples (r=0.97, p<0.0001). The pooled mRNA (BioChain) control specimen was included in triplicate within a single run to assess reproducibility and included in every run as a control pad to assess repeatability. A non-tumor lung sample was also repeated between runs to assess repeatability. 12 clinical samples were repeated twice under the same conditions 14 days apart. All positive results were concordant between duplicate samples. Excellent correlation was observed between gene log 2 ratio results for the duplicate samples repeated 14 days apart (r=0.99, p<0.0001).

Two different runs were performed with 11 repeat samples and 1 control using different Nanostring instruments and again using different PCR instruments. The different Nanostring instruments were located in the Moffitt Molecular Advanced Diagnostics (CLIA) and the Moffitt Molecular Research Core Laboratories. The PCR instruments were both in the Moffitt CLIA laboratory. Excellent correlation was observed with comparison of the PCR-to-PCR instrument gene log 2 ratio results (r=0.98, p<0.0001) and the Nanostring-to-Nanostring instrument results (r=0.98. p<0.0001).

A repeat run was performed with 11 clinical samples and 1 control with a different set of Nanostring reagents (different lot number). With the change in lot number, 4 of the 204 gene probes were also changed to assess the ability to change probes. This additional step of changing 4 probes helps assure that as clinical needs for new biomarker occur, probes may be changed with testing of duplicate samples before and after reagent or probe changes. Minimal lot-to-lot variability was detected with excellent correlation between gene log 2 ratio results for the duplicate samples (r=0.98, p<0.0001). Sample conditions may vary, and this could affect the accuracy of an assay. Specimens with potential interfering substances were included in testing. 4 lung cancer samples with anthracnosis (black pigment in lung caused by pollutant such as smoke), 2 melanoma samples with melanin, and 3 bone samples were included (Table 6). Results from samples collected within the past two years, 2020-2021 were compared to older samples, 2010-2019.

The lung cancer and melanoma samples had MET exon 14 skipping or gene upregulation by STAR NGS. Two log cancer samples that had MET exon 14 skipping detected by NGS were also positive for MET exon 14 by RNA STEP. In the lung cancer samples, gene upregulation (cut-off log 2ratio ≥2) was detected in 3 of 5 genes with increased copy number by STAR NGS (cut-off ≥5 copies). The two discordant results both had 5 copies near the cur-off for calling gene amplification. With a lower log 2 ratio cut-off of 1 for gene upregulation, all 5 genes would be called positive.

In the melanoma samples, gene upregulation (cut-off log 2 ratio ≥2) was detected in 5 of 8 genes with increased copy number by STAR NGS (cut-off ≥5 copies). A lower log 2 ratio of 1 for gene upregulation would have a PPA of 87.5% (7 of 8). The PPA was higher in the newer samples, 79.5% versus 65.2% even though the accuracy of samples collected in the past 2 years (2020-2021) versus older samples (2010-2019) was similar, 93.5% versus 92.8%, respectively. See Table 6 for comparison of PPA, NPA, PPV, NPV and accuracy by sample age.

The effect of background tissue type was evaluated on the accuracy of RNA STEP versus STAR NGS results. Our sample set included 21 lung, 15 liver, 8 brain, 7 head and neck, 7 lymph node, 8 ovary, 7 soft tissue, 6 skin, 4 peritoneum samples. The remaining tissue sites had 3 or fewer representative samples: pleural fluid (3), urinary bladder (3), rectum (2), breast (2), bone (1), colon (1), kidney (1), pancrease (1), prostate (1), and stomach (1). Accuracy was >909% for all tissue sites analyzed (Table 8) using ≥5 copies as the positivity cut-off for gene amplification by STAR NGS and a log 2 ratio ≥2.0 as the positivity cut-off for gene upregulation by RNA STEP. These results demonstrate that lung and melanoma samples may be accepted even with abundant arthrosis and melanin. Samples processed with decalcification or freezing are also acceptable. Older samples may be test up to 10 years old, but if older than 2 years should be reported with qualification that there may be a higher risk for false negative results.

For diagnostic validation, we analyzed mRNA samples extracted from 102 FFPE tissue samples collected from Moffitt Cancer Center patients with known MET exon 14 skipping status and gene amplification status for 59 genes by NGS. Of the 59 genes covered by NGS for gene copy number variants, 38 genes are also covered by RNA STEP for gene expression.

RNA STEP results from the clinical samples were compared with STAR NGS (CLIA) results for accuracy, positive percentage agreement (PPA), negative percentage agreement (NPA), positive predictive value (PPV), and negative predictive value (NPV). We specifically compared different log 2 ratio cut-offs (1, 2, and 3) for RNA STEP detection of MET exon 14 skipping in samples with MET exon 14 skipping reported by STAR NGS, RNA STEP accuracy, PPA, NPA, PPV, and NPV was evaluated for detection of gene upregulation in clinical samples with gene amplification identified by STAR NGS using cut-offs of 1, 1.5, and 2 for RNA STEP and cut-offs or >4 copies and >5 copies for NGS gene amplification

Comparison for results for gene amplification (cut-off: 5 copies) versus upregulation (cut-off: log 2 ratio ≥2) of the same gene in patient samples demonstrated an overall accuracy of 93%, despite biological differences between gene amplification and upregulation and that gene amplification is DNA-based detection. Accuracy was 93.0% (positive percentage agreement=69.4%; negative percentage agreement=93.8%; positive predictive value=27.1%; negative predictive value=98.9%) for all comparable gene amplification versus expression results (Table 9). See Attachment 2 for tables with different cut-offs. Higher PPA was observed for specific genes such as CDK4 (100%, N=10), ERBB2 (90.9%, N=11). MDM2 (85%. N=20), and MYC (87.5%, N=8). For the 11 ERBB2 cases, 7 had additional ERBB2 results by IHC, FISH, Foundation 1 solid tumor NGS, and/or Guardant 360 cfDNA NGS (Table 10). Of note, the case with a discordant result for STAR NGS versus RNA STEP was positive by HER2/neu IHC and likely a false negative RNA STEP result. This sample had the lowest tumor cellularity (30%) of the 11 samples and was a lymph node metastasis. The negative result may be due to dilution of the tumor RNA by non-tumor cell RNA and helps justify including a qualifying statement on the report for samples with lower tumor cellularity to explain the higher chance for false negative results.

The general negative predictive value (NPV) and negative percentage agreement (NPA) were high (both >90%), meaning that in general non-amplified genes did not have high gene expression and furthermore that if gene expression was not upregulated, gene amplification was unlikely. Upregulated gene expression generally did not predict gene amplification well as reflected by a low general positive predictive value (PPV) of 27.9%. An explanation for the overall low PPV might be that there are many causes for gene upregulation other than gene amplification.

Specific comparison of results of RNA STEP and STAR NGS for MET exon 14 skipping was performed for 102 cases (Table 11). STAR NGS was defined as MET exon 14 skipping positive if MET exon 14 skipping was present on the clinical STAR NGS report. Of the 102 STAR NGS reports reviewed. 10 were positive for MET exon 14 skipping. All 10 were concordantly positive for RNA STEP with a positive cut-off of log 2 ratio ≥2. The 10 positive samples had an average RNA STEP log 2 ratio of 4.9, ranging from 2.5 to 6.7. Both samples that did not have MET exon 14 skipping reported by NGS were lung adenocarcinomas with MET amplification. They had MET exon 14 skipping log 2 ratios of 2.1 and 2.8. The sample with the log 2 ratio of 2.8 also had an EGFR exon 19 deletion by STAR NGS. One possible explanation for the discordant STAR NGS (negative) and RNA STEP (positive) results is that this patient may have been beginning to develop evolutionary resistance to EGFR targeted therapy with an increase in MET exon 14 skipping, but still below the diagnostic threshold for STAR NGS. All other 90 samples were concordantly negative for MET exon 14 skipping by both RNA STEP and STAR NGS. Accuracy with a log 2 ratio positive cut-off of ≥2 was 98.0%. Orthogonal testing is recommended for MET exon 14 skipping detected with log 2 ratio between 2 and 4 prior to clinical action. A probe for the FSHR gene was added with the change in reagent lots because it is overexpressed in most ovarian cancer subtypes and may be a therapeutic target. FSHR gene upregulation was significantly higher in high grade serous ovarian cancer (HGSOC) versus other cancer types with a prevalence of 28.6% (6 of 21) in HGSOC versus 3.7% (2 of 54) in other cancer types (log 2 ratio cut-off of ≥2). With a lower log 2 ratio cut-off of ≥1, the positive FSHR prevalence in HGSOC versus other cancer types was 38.1% (8 of 21) versus 9.3% (5 of 54).

Results of RNA STEP and RNAseq were both performed on the same mRNA from 25 frozen lung squamous cell carcinomas. The results from the 25 samples for 191 genes covered by both assays, were compared by correlation analysis. Overall, the RNA STEP and RNAseq data were well correlated with a mean correlation of 0.68. Of the 191 genes analyzed, 92.1% (176/191) had a correlation p-value <0.05.

The reference range is the range of results expected in the healthy population. A commercial pooled RNA sample was used from 10 non-tumor tissues from healthy individuals and a non-tumor lung sample as our reference samples. The pooled RNA sample is included in every run with specific run results for the pooled control sample used to calculate the relative change in gene expression for each of the 204 genes in the patient samples. The range of gene expression results expected in the healthy population is a log 2 ratio between −2 and +2 relative to the pooled control sample (equivalent to less than a 4-fold difference between the sample and the control).

According to the College of American Pathologists, the reportable range of an assay is the range of values that the laboratory reports for that assay. Genes with an associated log 2 ratio ≥2 result will be reportable as positive for upregulation or MET exon 14 skipping. The report will list all of the genes covered by the assay in the below groups with their respective log 2 ratio results. Orthogonal validation of results prior to clinical action are recommended. The evidence to support the clinical utility for genes included in the assay with specific log 2 ratio cut-offs is expected to build with its use in prospective clinical trials.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

### Tables

TABLE 1
Gene Content in the Salah Targeted Expression Panel (STEP)

| | | | | | | | |
|---|---|---|---|---|---|---|---|
| ABRAXAS1 | BRIP1 | CHEK2 | FOLR1 | KDR | MRE11 | PDGFRB | STK11 |
| ACKR2 | BTN2A1 | CIITA | FSHR | KEAP1 | MS4A1 | PIK3CA | TACSTD2 |
| ACKR3 | BTN3A1 | CREBBP | GATA6 | KIT | MSH2 | PMS2 | TAFAZZIN |
| ACOT12 | CCND1 | CSF1R | GRB2 | KLRK1 | MSH6 | POLE | TERT |
| ACTA2 | CCNE1 | CT83 | GSK38 | KRAS | MTOR | POU2F3 | TFF1 |
| ADORA2A | CD14 | CTAG1A | HAVCR2 | LAG3 | MUC1 | PPP2R2A | TIGIT |
| AKT1 | CD274 | CTAGE1 | HDAC1 | LCK | MYC | PSCA | TMPRSS2 |
| AKT2 | CD33 | CTLA4 | HGF | MAGEA1 | NBN | PTCH1 | TNFRSF10A |
| AKT3 | CD3D | CTNNB1 | HLA-A | MAGEA10 | NCAM1 | PTEN | TNFRSF10B |
| ALK | CD3E | DDR2 | HLA-B | MAGEA3/6 | NEUROD1 | PTK7 | TP53 |
| ANPEP | CD3G | DLL3 | HLA-C | MAGEA4 | NF1 | PTP4A1 | ULBP1 |
| APC | CD4 | DUSP4 | HLA-DPA1 | MAGEB2 | NFE2L2 | PVR | VIM |
| AR | CD68 | E2F1 | HLA-DPB1 | MAGEC1 | NFKB1 | RAD51 | WEE1 |
| ARID1A | CD70 | EGFR | HLA-DQA1 | MAGEC2 | NFKB2 | RAD51B | WT1 |
| ASCL1 | CD80 | ERBB2 | HLA-DQA2 | MAP2K1 | NRAS | RAD51C | YAP1 |
| ATM | CD83 | ERBB3 | HLA-DQB1 | MAP2K2 | NRG1 | RAD51D | ABCF1 |
| ATR | CD86 | ERBB4 | HLA-DRA | MAPK1 | NTRK1 | RAD54L | DNAJC14 |
| AXL | CD8A | ESR1 | HLA-DRB1 | MAPK3 | NTRK2 | RAF1 | ERCC3 |
| B2M | CDH1 | EZH2 | HMMR | MCL1 | NTRK3 | RB1 | MRPL19 |
| BAG1 | CDH2 | FANCA | HRAS | MDM2 | OR5V1 | RET | OAZ1 |
| BARD1 | CDK12 | FGFR1 | IDH1 | MDM4 | PALB2 | RICTOR | POLR2A |
| BCL2 | CDK2 | FGFR2 | IDH2 | MET | PARP1 | ROS1 | PSMC4 |
| BCL2L11 | CDK4 | FGFR3 | IL11 | MET_e14_skip | PARP2 | SDC1 | SF3A1 |
| BIRC5 | CDK6 | FGFR4 | IL13RA2 | MICA | PCSK9 | SETD2 | TBC1D10B |
| BRAF | CDKN2A | FLT1 | IL2RB | MICB | PDCD1 | SIK1 | TBP |
| BRCA1 | CHD1 | FLT3 | IRS2 | MKI67 | PDCD1LG2 | SLC34A2 | TLK2 |
| BRCA2 | CHEK1 | FLT4 | JAK2 | MLH1 | PDGFRA | SMAD4 | TMUB2 |

TABLE 2
Quality Control Specifications for
RNA extracted from FFPE samples

| Quality Control | Quality Control Specification |
|---|---|
| RNA concentration | ≥37.5 ng/μL or ≥150 ng |
| RNA purity | A260/A280 ratio = 1.70-2.30 |

TABLE 3
LOD: analysis of 2 serially diluted samples (A,
80% tumor cellularity; B, 20% tumor cellularity
A. Sample P6-9 with 80% Tumor Cellularity

| | Log2 Ratio | | | |
|---|---|---|---|---|
| [RNA, | Met ex | | | |
| ng] | 14 skip | MDM2 | CDK4 | KRAS |
| 300 | 6.0, 5.1 | 4.3, 3.5, 4.2 | 5.8, 7.0, 5.8 | 2.6, 2.0, 2.4 |
| 200 | 5.2 | 3.5 | 6.9 | 1.9 |
| 100 | 5.1 | 3.4 | 6.8 | 1.9 |
| 50 | 5.2 | 3.4 | 6.5 | 2.0 |
| 25 | 5.8 | 4.1 | 5.8 | 2.3 |
| 10 | 5.9 | 4.0 | 5.5 | 2.3 |

B. Sample with 20% Tumor Cellularity

| | Log2 Ratio |
|---|---|
| [RNA, | Met ex |
| ng] | 14 skip |
| 300 | 4.23, 3.78, 3.35 |
| 200 | 3.45 |
| 100 | 3.37 |
| 50 | 3.51 |
| 25 | 3.79 |
| 10 | 3.06 |

TABLE 5A

| | | NGS | NanoString | [RNA, | |
|---|---|---|---|---|---|
| Sample ID | GENE | (Copies) | (Log2 Ratio) | ng] | % Tumor |
| P2-9 | KRAS | 10 | 2.39 | 158.3 | 20 |
| P2-10 | ERBB2 | 11 | −0.26 | 300 | 30 |
| P3-6 | EGFR | 8 | 3.24 | 300 | 30 |
| | CCND1 | 5 | 1.62 | | |
| P25-1 | EGFR | 8 | −4.49 | 13.2 | 20 |
| | CDK4 | 7 | 4.12 | | |
| P25-2 | ERBB2 | 8 | 3.85 | 300 | 20 |
| P25-3 | MDM2 | 9 | 3.24 | 123.6 | 10 |
| | CDK4 | 6 | 2.37 | | |
| P25-4 | ERBB2 | 6 | 2.95 | 72.4 | 30 |
| P25-5 | KRAS | 5 | 1.06 | 124 | 20 |
| | MYC | 13 | 4.15 | | |
| | ESR1 | 5 | −0.06 | | |

TABLE 5B

| Sample | | NGS | NanoString | [RNA, | % |
|---|---|---|---|---|---|
| ID | GENE | (reads) | (Log2 Ratio) | ng] | Tumor |
| P1-1 | Met ex14 skip | 393 | 4.23 | 300 | 20 |
| P1-5 | Met ex14 skip | 167 | 4.43 | 227.1 | 30 |
| P7-3 | Met ex14 skip | 4495 | 6.66 | 300 | 30 |

TABLE 6
Samples tested to evaluate for interfering substances

| | | | | | | NGS | | |
|---|---|---|---|---|---|---|---|---|
| | % | [RNA, | | Tissue | | (copies/ | NS (Log2 | |
| Sample # | Tumor | ng] | Diagnosis | Site | Gene | reads) | Ratio) | Notes |
| 1 | 40 | 300 | Lung | Lung | KRAS | 5 | 1.46 | Mild |
| | | | adenocarcinoma | | CDK4 | 5 | 3.03 | anthracosis |
| | | | | | MDM2 | 11 | 3.74 | |
| | | | | | Met e14 skip | 163 | 4.28 | |
| 2 | 70 | 300 | Lung squamous | Lung | PIK3CA | 5 | 1.95 | Moderate |
| | | | cell carcinoma | | | | | anthracosis |
| 3 | 60 | 300 | Lung squamous | Lung | MDM2 | 5 | 2.13 | Moderate |
| | | | cell carcinoma | | | | | anthracosis |
| 4 | 60 | 300 | Lung | Lung | Met e14 skip | 1739 | 5.62 | Mild |
| | | | Adenocarcinoma | | | | | anthracosis |
| 5 | 80 | 279 | Mucosal | Head | KIT | 6 | 1.9 | Melanin in |
| | | | lentiginous | | MDM2 | 12 | 4.12 | lymph node |
| | | | melanoma | | PDGFRA | 5 | −0.61 | |
| | | | | | CDK4 | 8 | 5.01 | |
| | | | | | RICTOR | 11 | 1.11 | |
| 6 | 70 | 298 | Cutaneous | Lymph | MDM2 | 20 | 6.53 | Abundant |
| | | | melanoma | node | KRAS | 15 | 5.21 | melanin |
| | | | | | CDK4 | 13 | 6.75 | |
| 7 | 50 | 300 | Lung | Bone | MET | 6 | 2.22 | |
| | | | adenocarcinoma | | | | | |
| 8 | 10 | 123.6 | Metastatic | Bone | MDM2 | 9 | 3.24 | |
| | | | carcinoma, favor | | | | | |
| | | | adenocarcinoma | | CDK4 | 6 | 2.37 | |
| 9 | 20 | 124 | Metastatic | Bone | KRAS | 5 | 1.06 | |
| | | | squamous cell | | MYC | 13 | 4.15 | |
| | | | carcinoma | | ESR1 | 5 | −0.06 | |

TABLE 7A
PPA, NPA, PPV, NPV, and accuracy for
recent samples (2020-2021, N = 36)

| 2020-2021 | Copy | Copy | |
|---|---|---|---|
| Samples | Number ≥5 | Number <5 | |
| Log2 Ratio ≥2.0 | 37 | 75 | PPV = 33.0% |
| Log2 Ratio <2.0 | 12 | 1208 | NPV = 99.0% |
| | PPA = | NPA = | Accuracy = |
| | 75.5% | 94.2% | 93.5% |

TABLE 7B
PPA, NPA, PPV, NPV, and accuracy for
older samples (2010-2019, N = 66)

| 2010-2019 | Copy | Copy | |
|---|---|---|---|
| Samples | Number ≥5 | Number <5 | |
| Log2 Ratio ≥2.0 | 47 | 151 | PPV = 23.7% |
| Log2 Ratio <2.0 | 25 | 2219 | NPV = 98.9% |
| | PPA = | NPA = | Accuracy = |
| | 65.3% | 93.6% | 92.8% |

TABLE 8

| | | | | | Lymph | | | | Soft |
|---|---|---|---|---|---|---|---|---|---|
| | Brain | Head | Liver | Lung | Nodes | Ovary | Peritoneum | Skin | Tissue |
| PPV | 36% | 36.4% | 22.7% | 20.0% | 35.3% | 10% | 50% | 21.1% | 30.0% |
| NPV | 99.3% | 97.9% | 99.2% | 98.6% | 87.7% | 100% | 99.4% | 99.0% | 99.2% |
| PPA | 81.8% | 61.5% | 71.4% | 58.3% | 50.0% | 100% | 90% | 66.7% | 75.0% |
| NPA | 94.4% | 94.3% | 93.7% | 92.6% | 95.6% | 93.9% | 94.9% | 93.1% | 94.4% |
| Accuracy | 93.9% | 92.7% | 93.2% | 91.5% | 93.4% | 93.9% | 94.6% | 92.3% | 93.8% |
| n | 8 | 7 | 15 | 21 | 7 | 8 | 5 | 6 | 7 |

TABLE 9
Summary of concordance of RNA STEP and STAR NGS results for
38 mutually covered genes in 102 independent samples for gene
upregulation (RNA STEP) versus amplification (STAR NGS).

| | | STAR NGS | | |
|---|---|---|---|---|
| | All 102 Samples/ | Amplified (Copy | Not (Copy | |
| | All wild type genes | Number ≥5) | Number <5) | |
| RNA STEP | Upregulated | 84 | 226 | PPV = 27.1% |
| | (Log2 Ratio ≥2.0) | | | |
| | Not (Log2 Ratio <2.0) | 37 | 3427 | NPV = 98.9% |
| | | PPA = 69.4% | NPA = 93.8% | Accuracy = 93.0% |

TABLE 10
HER2 positive cases by STAR NGS and tested with RNA STEP

| STAR | | | | | | |
|---|---|---|---|---|---|---|
| NGS | NanoString | | | | % | |
| (copies) | (log2 ratio) | IHC/FISH | NGS (F1 /G360) | [RNA, ng] | Tumor | Diagnosis |
| 6 | 4.99 | Not | Not tested | 300 | 50 | Bladder urothelial |
| | | tested | | | | carcinoma |
| 11 | −0.26 | THC 3+ | Not tested | 300 | 30 | Poorly differentiated |
| | | | | | | carcinoma of unknown |
| | | | | | | primary |
| 5 | 2.69 | Not | Not tested | 300 | 80 | Upper tract urothelial |
| | | tested | | | | carcinoma |
| 11 | 5.76 | Not | Not tested | 300 | 85 | Ovarian bigh grade |
| | | tested | | | | serous carcinoma |
| 9 | 4.93 | Not | F1 negative | 300 | 70 | Rectal |
| | | tested | (different sample) | | | adenocarcinoma |
| 10 | 5.15 | Not | F1 positive, 125 | 300 | 65 | Stomach |
| | | tested | copies; G360 | | | adenocarcinoma |
| | | | positive | | | |
| 11 | 5.46 | IHC 3+ | Not tested | 215.4 | 40 | Breast invasive ductal |
| | | | | | | carcinoma |
| 7 | 5.19 | IHC 3+ | Not tested | 300 | 70 | Ceruminous gland |
| | | | | | | adenocarcinoma |
| 12 | 5.78 | Not | G360 high, 14.7 | 243.92 | 65 | Non-small cell lung |
| | | tested | copies | | | carcinoma (NSCLC) |
| 5 | 3.42 | Not | Not tested | 300 | 70 | Gallbladder adenocarcinoma |
| | | tested | | | | |
| 8 | 5.36 | IHC 3+; | Not tested | 193.40 | 60 | Breast invasive carcinoma, |
| | | FISH | | | | NOS |
| | | positive | | | | |

TABLE 11

| | All Samples/MET | STAR NGS | | |
|---|---|---|---|---|
| | ex14 | MET ex14 | MET ex14 | |
| | analysis | positive | negative | |
| RNA STEP | Positive (Log2 | 10 | 2 | PPV = 83.3% |
| | Ratio ≥2.0) | | | |
| | Negative (Log2 | 0 | 90 | NPV = 100% |
| | Ratio <2.0) | | | |
| | | PPA = 100% | NPA = 97.8% | Accuracy = 98.0% |

TABLE 14

| | | Cellu- | | Tissue | Peptide | LC-MS/MS | LC-MS/MS |
|---|---|---|---|---|---|---|---|
| Sample | | larity | Pathologist's | Area | Recovery | Peptide | Protein |
| ID | Pathology Evaluation | (%) | Comments | (mm²) | (μg) | IDs | IDs |
| 1 | Squamous Cell Carcinoma (moderately | 30 | large, LCM Recommended | 192.5 | 66.8 | 17365 | 3126 |
| | differentiated) | | | | | | |
| 2 | Squamous Cell Carcinoma (moderately | 15 | LCM Recommended | 73.9 | 32.6 | 13208 | 2569 |
| | differentiated) | | | | | | |
| 3 | Squamous Cell Carcinoma (poorly differentiated) | 90 | tiny | 3.3 | 9.0 | 14544 | 2805 |
| 4 | Squamous Cell Carcinoma (poorly differentiated) | 40 | | 5.7 | 10.5 | 15398 | 2920 |
| 5 | Adenocarcinoma (poorly differentiated) | 90 | large | 430.5 | 167.0 | 13689 | 2739 |
| 6 | Adenocarcinoma (moderately differentiated) | 20 | LCM Recommended | 154.4 | 58.5 | 17041 | 3040 |
| 7 | Large Cell Carcinoma (poorly differentiated) | 70 | small, | 9.1 | 23.4 | 17520 | 3135 |
| 8 | Adenosquamous Carcinoma (poorly | 20 | Frozen First, Then FFPE | 78.8 | 120.3 | 14837 | 2794 |
| | differentiated) | | | | | | |
| 9 | Adenocarcinoma (well to moderately | 35 | small | 12.0 | 18.5 | 14648 | 2786 |
| | differentiated) | | | | | | |
| 10 | Squamous Cell Carcinoma (moderately | 90 | small, bloody | 69.7 | 52.0 | 8391 | 2041 |
| | differentiated) | | | | | | |
| 11 | Adenocarcinoma (poorly differentiated) | 35 | small | 22.1 | 19.3 | 11881 | 2376 |
| 12 | NSCLC with features of Adenocarcinoma (poorly | 30 | LCM Recommended | 17.4 | 23.2 | 17660 | 3170 |
| | differentiated) | | | | | | |
| 13 | Squamous Cell Carcinoma (poorly differentiated) | 20 | Frozen First, Then FFPE | 29.0 | 25.5 | 15900 | 2976 |
| 14 | Adenocarcinoma (moderately differentiated) | 50 | | 76.2 | 55.5 | 18287 | 3221 |
| 15 | Adenocarcinoma (poorly differentiated) | 70 | Necrosis, LCM | 151.8 | 133.4 | 16353 | 3034 |
| | | | Recommended | | | | |
| 16 | Adenocarcinoma (moderately differentiated) | 20 | LCM Recommended | 55.5 | 16.4 | 15674 | 2964 |
| 17 | Squamous Cell Carcinoma (moderately | 75 | small | 35.8 | 28.4 | 17344 | 3126 |
| 18 | Adenocarcinoma (moderately differentiated) | 20 | scattered | 348.0 | 136.7 | 17069 | 2914 |
| 19 | Squamous Cell Carcinoma (moderately to poorly | 90 | | 40.5 | 24.9 | 16879 | 3089 |
| | differentiated) | | | | | | |
| 20 | Squamous Cell Carcinoma (moderately | 85 | | 23.7 | 13.9 | 17086 | 3059 |
| | differentiated) | | | | | | |
| 21 | Adenocarcinoma (poorly differentiated ) | 90 | | 91.9 | 64.5 | 18040 | 3194 |
| 22 | Adenocarcinoma (well differentiated) | 60 | | 164.1 | 43.5 | 15933 | 2943 |
| 23 | Adenocarcinoma (poorly differentiated) | 50 | small | 30.9 | 25.8 | 16277 | 2951 |
| 24 | NSCLC favoring Squamous Cell Carcinoma | 50 | small | 24.0 | 24.3 | 18229 | 3129 |
| | (poorly differentiated) | | | | | | |
| 25 | Adenocarcinoma (poorly differentiated) | 90 | | 32.3 | 15.1 | 15051 | 2886 |
| 26 | Adenocarcinoma (moderately differentiated) | 60 | poor H&E staining | 203.6 | 44.5 | 14628 | 2801 |
| 27 | Adenocarcinoma (moderately to poorly | 70 | | 25.1 | 19.3 | 16095 | 3112 |
| | differentiated) | | | | | | |
| 28 | Adenocarcinoma (poorly differentiated) | 80 | | 122.9 | 116.6 | 13542 | 2815 |
| 29 | Squamous Cell Carcinoma (poorly differentiated) | 50 | | 8.4 | 18.0 | 14903 | 2989 |
| 30 | Adenocarcinoma (well to moderately | 50 | tiny | 3.9 | 7.5 | 8732 | 2198 |
| | differentiated) | | | | | | |

1. A genomic assay panel for cancer diagnosis and clinical trial relevance comprising 204 genes selected from the group consisting of ABRAXAS1, ACKR2, ACKR3, ACOT12, ACTA2, ADORA2A, AKT1, AKT2, AKT3, ALK, ANPEP, APC, AR, ARIDIA, ASCL1, ATM, ATR, AXL, B2M, BAG1, BARD1, BCL2, BCL2L11, BIRC5, BRAF, BRCA1, BRCA2, BRIP1, BTN2A1, BTN3A1, CCND1, CCNE1, CD14, CD274, CD33, CD3D, CD3E, CD3G, CD4, CD68, CD70, CD80, CD83, CD86, CD8A, CDH1, CDH2, CDK12, CDK2, CDK4, CDK6, CDKN2A, CHD1, CHEK1, CHEK2, CUTA, CREBBP, CSF1R, CT83, CTAGlA, CTAGE1, CTLA4, CTNNB1, DDR2, DLL3, DUSP4, E2F1, EGFR, ERBB2, ERBB3, ERBB4, ESR1, EZH2, FANCA, FGFR1, FGFR2, FGFR3, FGFR4, FLT1, FLT3, FLT4, FOLR1, FSHR, GATA6, GRB2, GSK3B, HAVCR2, HDAC1, HGF, HLA-A, HLA-B, HLA-C, HLA-DPA1, HLA-DPB1, HLA-DQA1, HLA-DQA2, HLA-DQB1, HLA-DRA, HLA-DRB1, HMMR, HRAS, IDH1, IDH2, IL11, IL13RA2, IL2RB, IRS2, JAK2, KDR, KEAP1, KIT, KLRK1, KRAS, LAG3, LCK, MAGEA1, MAGEA10, MAGEA3/6, MAGEA4, MAGEB2, MAGEC1, MAGEC2, MAP2K1, MAP2K2, MAPK1, MAPK3, MCL1, MDM2, MDM4, MET, MET_el4_skip, MICA, MICB, MKI67, MLH1, MRE11, MS4A1, MSH2, MSH6, MTOR, MUC1, MYC, NBN, NCAM1, NEUROD1, NF1, NFE2L2, NFKB1, NFKB2, NRAS, NRG1, NTRK1, NTRK2, NTRK3, OR5V1, PALB2, PARP1, PARP2, PCSK9, PDCD1, PDCD1LG2, PDGFRA, PDGFRB, PIK3CA, PMS2, POLE, POU2F3, PPP2R2A, PSCA, PTCH1, PTEN, PTK7, PTP4A1, PVR, RAD51, RAD51B, RAD51C, RAD51D, RAD54L, RAF1, RB1, RET, RICTOR, ROS1, SDC1, SETD2, SIK1, SLC34A2, SMAD4, STK11, TACSTD2, TAFAZZIN, TERT, TFF1, TIGIT, TMPRSS2, TNFRSF10A, TNFRSF10B, TP53, ULBP1, VIM, WEE1, WT1, and/or YAP1.

2. The genomic assay panel of claim 1, wherein the panel further comprises one or more housekeeping genes selected from ABCF1, DNAJC14, ERCC3, MRPL19, OAZ1, POLR2A, SMC4, SF3A1, TBClD10B, TBP, TLK2, and/or TMUB2.

3. The genomic assay panel of claim 1, wherein the expression panel comprises an RNA expression panel.

4. The genomic assay panel of claim 3, wherein the expression panel comprises an RNA Salah Targeted Expression Panel (STEP).

5. A proteomic assay panel for cancer diagnosis and clinical trial relevance comprising the peptide targets comprising ACTA_VAPEEHPTLLTEAPLNPK (SEQ ID NO: 1), ACTB_AGFAGDDAPR (SEQ ID NO: 2), AKT1_SLLSGLLK (SEQ ID NO: 3), AKT2_EGISDGATMK (SEQ ID NO: 4), AKT2_SLLAGLLK (SEQ ID NO: 5), AKT3_TDGSFIGYK (SEQ ID NO: 6), ALBU_LVNEVTEFAK (SEQ ID NO: 7), ALK_DPEGVPPLLVSQQAK (SEQ ID NO: 8), ALK_NCLLTCPGPGR (SEQ ID NO: 9), ARAF_GLNQDCCVVYR (SEQ ID NO: 10), ARAF_IGTGSFGTVFR (SEQ ID NO: 11), BCL2_FATVVEELFR (SEQ ID NO: 12), BRAF_GLIPECCAVYR (SEQ ID NO: 13), BT2A1_DPYGGVAPALK (SEQ ID NO: 14), BT3A1_TANPILLVSEDQR (SEQ ID NO: 15), CADH1_NDVAPTLMSVPR (SEQ ID NO: 16), CADH1_TAYFSLDTR (SEQ ID NO: 17), CADH2_GPFPQELVR (SEQ ID NO: 18), CADH2_LSDPANWLK (SEQ ID NO: 19), CCND1_AEETCAPSVSYFK (SEQ ID NO: 20), CCND1_AYPDANLLNDR (SEQ ID NO: 21), CD20_AHTPYINIYNCEPANPSEK (SEQ ID NO: 22), CD3D_LSGAADTQALLR (SEQ ID NO: 23), CD3E_GSKPEDANFYLYLR (SEQ ID NO: 24), CD4_ILGNQGSFLTK (SEQ ID NO: 25), CD80_ADFPTPSISDFEIPTSNIR (SEQ ID NO: 26), CD8A_AAEGLDTQR (SEQ ID NO: 27), CDK2_DLKPQNLLINTEGAIK (SEQ ID NO: 28), CDK2_VVPPLDEDGR (SEQ ID NO: 29), CDK4_DLKPENILVTSGGTVK (SEQ ID NO: 30), CDK4_VPNGGGGGGGLPISTVR (SEQ ID NO: 31), CDK6_ILDVIGLPGEEDWPR (SEQ ID NO: 32), CDN2A_ALLEAGALPNAPNSYGR (SEQ ID NO: 33), CDN2A_LPVDLAEELGHR (SEQ ID NO: 34), CHK1_DIKPENLLLDER (SEQ ID NO: 35), CTG1B_ASGPGGGAPR (SEQ ID NO: 36), CTNB1_AIPELTK (SEQ ID NO: 37), EGFR_GSTAENAEYLR (SEQ ID NO: 38), EGFR_IPLENLQIIR (SEQ ID NO: 39), ERBB2_ELVSEFSR (SEQ ID NO: 40), G3P_VGVNGFGR (SEQ ID NO: 41), GSK3B_LLEYTPTAR (SEQ ID NO: 42), HBA_VGAHAGEYGAEALER (SEQ ID NO: 43), HBB_VNVDEVGGEALGR (SEQ ID NO: 44), I13R2_FPYLEASDYK (SEQ ID NO: 45), K2C5_LAELEEALQK (SEQ ID NO: 46), K2C6A_SGFSSVSVSR (SEQ ID NO: 47), K2C7_SAYGGPVGAGIR (SEQ ID NO: 48), KI67_SGASEANLIVAK (SEQ ID NO: 49), KIT_LLCTDPGFVK (SEQ ID NO: 50), KKLC1_LVELEHTLLSK (SEQ ID NO: 51), KRAS_SFEDIHHYR (SEQ ID NO: 52), LMNA_SGAQASSTPLSPTR (SEQ ID NO: 53), LMNA_VAVEEVDEEGK (SEQ ID NO: 54), MAGA1_VADLVGFLLLK (SEQ ID NO: 55), MAGA3_LLTQHFVQENYLEYR (SEQ ID NO: 56), MAGA4_EHTVYGEPR (SEQ ID NO: 57), MAGA6_LLTQYFVQENYLEYR (SEQ ID NO: 58), MAGAA_VTDLVQFLLFK (SEQ ID NO: 59), MAGB2_SGSLVQFLLYK (SEQ ID NO: 60), MAGC1_YTGYFPVIFR (SEQ ID NO: 61), MAGC2_VAELVEFLLLK (SEQ ID NO: 62), MCL1_LLFFAPTR (SEQ ID NO: 63), MET_TEFTTALQR (SEQ ID NO: 64), MET_VADFGLAR (SEQ ID NO: 65), MICA_EGLHSLQEIR (SEQ ID NO: 66), MKO1_GQVFDVGPR (SEQ ID NO: 67), MK03_GQPFDVGPR (SEQ ID NO: 67), MK03_NYLQSLPSK (SEQ ID NO: 68), MP2K1_IPEQILGK (SEQ ID NO: 69), MP2K1_VSHKPSGLVMAR (SEQ ID NO: 70), MP2K2_ISELGAGNGGVVTK (SEQ ID NO: 71), MTOR_LFDAPEAPLPSR (SEQ ID NO: 72), MTOR_VLGLLGALDPYK (SEQ ID NO: 73), NTRK2_SNEIPSTDVTDK (SEQ ID NO: 74), NTRK3_NCLVGANLLVK (SEQ ID NO: 75), NTRK3_VVSLEEPELR (SEQ ID NO: 76), Pan-RAS_LVVVGAGGVGK (SEQ ID NO: 77), PARP1_VFSATLGLVDIVK (SEQ ID NO: 78), PARP1_VVSEDFLQDVSASTK (SEQ ID NO: 79), PARP2_AEGLLQGK (SEQ ID NO: 80), PCNA_CAGNEDIITLR (SEQ ID NO: 81), PD1L1_LQDAGVYR (SEQ ID NO: 82), PD1L2_ATLLEEQLPLGK (SEQ ID NO: 83), PGFRA_FQTIPFNVYALK (SEQ ID NO: 84), PGFRB_GFSGIFEDR (SEQ ID NO: 85), PK3CA_LFQPFLK (SEQ ID NO: 86), PK3CA_LINLTDILK (SEQ ID NO: 87), PSCA_AVGLLTVISK (SEQ ID NO: 88), PTEN_GVTIPSQR (SEQ ID NO: 89), PTEN_IYSSNSGPTR (SEQ ID NO: 90), RAF1_GLQPECCAVFR (SEQ ID NO: 91), RAF1_GYASPDLSK (SEQ ID NO: 92), RAF1_VVDPTPEQFQAFR (SEQ ID NO: 93), RAS Mt_LVVVGACGVGK (SEQ ID NO: 94), RASH_SFEDIHQYR (SEQ ID NO: 95), RASN_SFADINLYR (SEQ ID NO: 96), RET_LLEGEGLPFR (SEQ ID NO: 97), RET_VFLSPTSLR (SEQ ID NO: 98), ROS1_IQDQLQLFR (SEQ ID NO: 99), TACD2_AAGDVDIGDAAYYFER (SEQ ID NO: 100), TBB1_EVDQQLLSVQTR (SEQ ID NO: 101), TBB1_GASALQLER (SEQ ID NO: 102), TBB5_ISVYYNEATGGK (SEQ ID NO: 103), TENA_VATYLPAPEGLK (SEQ ID NO: 104), TR10A_IQDLLVDSGK (SEQ ID NO: 105), TR10B_LLVPANEGDPTETLR (SEQ ID NO: 106), UFO_APLQGTLLGYR (SEQ ID NO: 107), UFO_TATITVLPQQPR (SEQ ID NO: 108), VGFR1_AVSSFPDPALYPLGSR (SEQ ID NO: 109), VIME_ILLAELEQLK (SEQ ID NO: 110), VIME_SLYASSPGGVYATR (SEQ ID NO: 111), and/or WEE1_SPTEPGPER (SEQ ID NO: 112).

6. A method of measuring the suitability of a patient for a treatment regimen or clinical trial comprising:
a. obtaining a tissue sample from the subject;
assaying gene expression in a tumor cell in the biological sample using the gene expression panel of claim 1 c.

7. The method of claim 6, wherein the gene expression panel is measured using a multiplexed polymerase chain reaction assay on the expression panel.

8. The method of claim 6, wherein the gene expression panel is measured using nanostring RNA expression profiling.

9. The method of claim 6, wherein protein expression is measured mass spectrometry.

10. The method of claim 9, wherein the mass spectrometry method comprises liquid chromatography multiple reaction monitoring (LC_MRM).

11. The method of claim 6, further comprising treating a subject for cancer.

12. A method of measuring the suitability of a patient for a treatment regimen or clinical trial comprising:
a. obtaining a tissue sample from the subject;
b. assaying the protein expression of in a tumor cell in the biological sample using protein expression panel of claim 5.

13. The method of claim 12, wherein the gene expression panel is measured using a multiplexed polymerase chain reaction assay on the expression panel.

14. The method of claim 12, wherein the gene expression panel is measured using nanostring RNA expression profiling.

15. The method of claim 12, wherein protein expression is measured mass spectrometry.

16. The method of claim 9, wherein the mass spectrometry method comprises liquid chromatography multiple reaction monitoring (LC_MRM).

17. The method of claim 12, further comprising treating a subject for cancer.
